(19) United States
(12) Patent Application Publication
(10) Pub. No.: US 2010/0256333 A1
(43) Pub. Date: Oct. 7, 2010

(54) Peptide-Based Beta Turn Mimetics

(75) Inventors: Kaidong Zhang, Rochester, NY (US); Frank Schweizer, Winnipeg (CA)

Correspondence Address: FULBRIGHT & JAWORSKI L.L.P., 600 CONGRESS AVE., SUITE 2400, AUSTIN, TX, 78701, US

(73) Assignee: UNIVERSITY OF MANITOBA, Winnipeg (CA)

(21) Appl. No.: 12/752,810

(22) Filed: Apr. 1, 2010

Related U.S. Application Data
(60) Provisional application No. 61/166,586, filed on Apr. 3, 2009.

(51) Int. Cl. C07K 9/00; C07H 7/06

(52) U.S. Cl. 530/322; 536/53

(57) ABSTRACT

Beta-mimetic compositions and methods of making and using such compositions in preparing bioactive peptides, such as antimicrobial peptides, are disclosed. In particular, spirocyclic proline hybrids are provided that may be used to alter the cis/trans isomerization of proline in a peptide, and which may replace a residue, for example, the i+2 residue, of a beta-turn in a peptide of known sequence, thereby retaining or modifying the structure of the peptide.

### CROSS-REFERENCE TO RELATED APPLICATIONS

This application claims priority to U.S. Provisional Patent Application Ser. No. 61/166,586, filed on Apr. 3, 2009, which is incorporated herein by reference.

### BACKGROUND OF THE INVENTION

The present invention relates generally to peptide and peptidomimetic drug development. More particularly, it concerns improvements in duplicating secondary structures in peptides that may be useful as therapeutic agents.

### SUMMARY OF THE INVENTION

The present invention stems, in part, from the discovery by the inventors that spirocyclic proline hybrids disclosed herein may be employed to generate beta-turn structures in peptides or peptidomimetic compounds of interest. In particular, spirocyclic proline hybrids are provided which may be used to alter the cis/trans isomerization of proline in a peptide, and which may replace a residue, for example, the i+2 residue, of a beta-turn in a peptide of known sequence, thereby retaining or modifying the structure of the peptide. Accordingly, the present invention provides beta-mimetic compositions and methods of making and using such compositions in preparing bioactive peptides, such as antimicrobial peptides.

In some embodiments, the present invention provides a compound of formula (I):

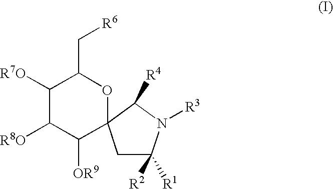

wherein one of R¹ and R² is —CH₂OH or CH₂OR⁵, wherein R⁵ is a hydroxy protecting group, and the other of R¹ and R² is —H; R³ and R⁴ are, independently, —H, —COOMe, —CONHMe, an amine protecting group, an amino acid, a protected amino acid, or a peptide; and R⁶ is —H, —OH, —NH₂, —N₃, a protected hydroxyl group, or a protected amine group; R⁷ through R⁹ are independently —H or R⁵, wherein R⁵ is a hydroxy protecting group. In these embodiments, R⁵ may be a hydroxy protecting group selected from an alkyl group, an alkenyl group, an alkanoyl group; an alkoxycarbonyl group; an alkenyloxycarbonyl group, an arylalkoxycarbonyl group, a nitrobenzyloxycarbonyl group, a trialkylsilyl group, or an aryl-alkyl group. By way of nonlimiting example, R⁵ may be a methoxymethyl group.

Certain embodiments of the present invention provide a compound of formula (I) that is a beta-turn mimetic compound. A beta-turn-mimetic of formula (I) as disclosed herein, may comprise a peptide at either R³ or R⁴ that comprises 10 or less amino acids, or 6 or less amino acids. Such a beta-turn-mimetic of formula (I) as disclosed herein, may comprise a peptide at both R³ and R⁴, or a beta-turn-mimetic may comprise an R⁴ that is a protected amino acid and an R³ that is a dipeptide.

In select embodiments, a beta-turn-mimetic of formula (I) is further defined as an analog of a bioactive peptide, such as, for example, an antimicrobial peptide preferably comprising a D- or L-proline unit. Exemplary antimicrobial peptides may include, but are not limited to, is a gramicidin, a tachyplesin, an indolicidin, an arenicin, a tritrpticin, or a tigerinin.

In an aspect, a compound of formula (I) may be further defined as a beta-turn mimetic compound in which R¹⁵ is an amino acid or a protected amino acid and R¹⁷ is a dipeptide. In some embodiments, a beta-turn mimetic is further defined as one of the following:

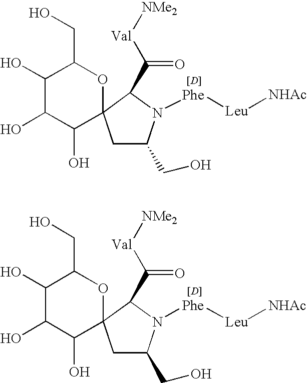

Select embodiments of the present invention provide a method of mimicking a beta-turn in a peptide comprising replacing an amino acid within a native beta-turn structure of the peptide with a spirocyclic proline hybrid formula (II):

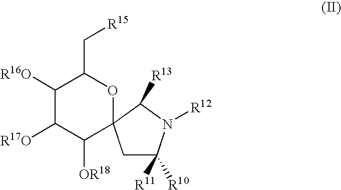

wherein at least one of R¹⁰ and R¹¹ is —CH₂OH or CH₂OR¹⁴, where R¹⁴ is a hydroxy protecting group; R¹² and R¹³ are, independently, —H, —COOH, —COOMe, —CONHMe, an amine protecting group, or a carboxy protecting group; and R¹⁵ is —H, —OH, —NH₂, —N₃, a methoxymethyl ether, a protected hydroxyl group, or a protected amine group; R¹⁶ through R¹⁸ are independently —H or R¹⁴ where R¹⁴ is a hydroxy protecting group. In these embodiments, R¹⁴ may be a hydroxy protecting group selected from an alkyl group, an alkenyl group, an alkanoyl group; an alkoxycarbonyl group; an alkenyloxycarbonyl group, an aryl-alkoxycarbonyl group, a nitrobenzyloxycarbonyl group, a trialkylsilyl group, and aryl-alkyl group. In particular, R¹⁴ may be a methoxymethyl group.

In select embodiments, a compound of formula (I) may be further defined as an analog of a bioactive peptide, such as, for example, an antimicrobial peptide preferably comprising a D- or L-proline unit. Exemplary antimicrobial peptides may include, but are not limited to, is a gramicidin, a tachyplesin, an indolicidin, an arenicin, a tritrpticin, or a tigerinin.

Some embodiments of the present invention method of synthesizing a beta-turn mimetic of claim 1, comprising blocking the hydroxyl and amine groups of a spirocyclic proline hybrid of formula (II):

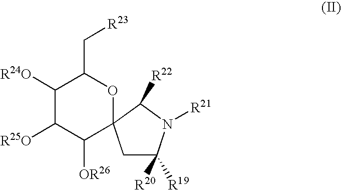

wherein one of R¹⁹ and R²⁰ is —CH₂OH and the other of R¹⁹ and R²⁰ is —H; R²¹ and R²² are, independently, —H, —COON, —COOMe, —CONHMe, an amine protecting group, or a carboxy protecting group; and R²³ is —H, —OH, —NH₂, —N₃, a methoxymethyl ether, a protected hydroxyl group, or a protected amine group; R²⁴ through R²⁶ are, independently, —H or a hydroxy protecting group; In these embodiments, the method further comprises hydrolyzing or displacing the carboxy terminal ester of hydroxyproline; optionally reacting the hydrolyzed carboxy terminal group with a protected amino acid or a peptide; deblocking the nitrogen of the hydroxyproline ring; optionally acetylating the nitrogen of the hydroxyproline ring, or coupling the nitrogen of the hydroxyproline ring to a protected amino acid or a peptide; and optionally deblocking one or more protected amino acids or hydroxyl groups.

Embodiments discussed in the context of methods and/or compositions of the invention may be employed with respect to any other method or composition described herein. Thus, an embodiment pertaining to one method or composition may be applied to other methods and compositions of the invention as well.

As used herein, “β-turn” and “beta-turn” refer to a structure characterized by the close approach of two C^{α} atoms (<7 Å), and wherein the corresponding residues are involved in hydrogen bond(s) in which the donor and acceptor residues are separated by about three residues (i-i₁₊₃ hydrogen bonding). The term “beta-turn” may also be used herein to refer to any one of the nine different classifications of beta-turn structures characterized by Hutchinson et al. (Protein Science, 3(12):2207-2216 (1994)), which is incorporated herein by reference.

“Peptide” refers to two or more amino acids joined together by an amide bond. The term “amino acid” may be used to refer to unnatural amino acids, too, such that the definition of β-amino acid would capture β-versions of unnatural amino acids.

The term “peptidomimetic” is used herein to mean a molecule designed to be structurally similar to and mimic the key portions of a natural peptide. A peptidomimetic may, for example, permit molecular interactions similar to a natural peptide molecule.

The term “beta-turn mimetic” incorporates the above definitions, wherein a compound mimics a beta-turn structure.

It is contemplated that any embodiment discussed in this specification can be implemented with respect to any method or composition of the invention, and vice versa. Furthermore, compositions of the invention can be used to achieve methods of the invention.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Any embodiment of any of the present methods, devices, and systems may consist of or consist essentially of—rather than comprise/include/contain/have—the described steps and/or features. Thus, in any of the claims, the term “consisting of” or “consisting essentially of” may be substituted for any of the open-ended linking verbs recited above, in order to change the scope of a given claim from what it would otherwise be using the open-ended linking verb.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Structure of spirocyclic glucose-3(S)-hydroxy-5′-hydroxymethyl)proline hybrids (Glc3′(S)-5′(CH₂OH)HypHs).

FIG. 2. Peptide mimics 3-6. C-terminal esters 3 and 4 are glucose-3′(S)-hydroxy-5′(S)-hydroxymethyl proline analogue and glucose-3′(S)-hydroxy-5′(R)-hydroxymethyl proline analogue while esters 5 and 6 serve as reference compounds.

FIG. 3. Assignment of cis and trans isomers in compounds 3 and 4 in CD₃OD through 1D nOe

FIG. 4. An exemplary illustration of the n→π* interaction (looking down C^{α}—N bond).

FIG. 5. Various N-Acetyl N′-methylamides. Compounds 9-13 have been previously synthesized^{9,31}

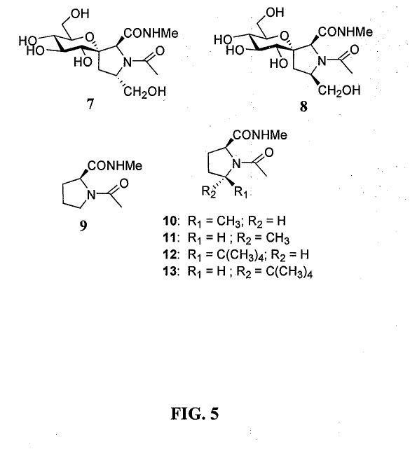

FIG. 6. Eyring Plots: trans-to-cis (up); cis-to-trans (down); compounds 3 (▪) 4 (▴), 5 (∘) and 6 (x).

FIG. 7. Van't Hoff plots for compounds 3 (▪), 4 (▴), 5 (∘) and 6 (♦) in D₂O.

FIG. 8. C^{β}-exo conformations of compounds 3 and 4. For clarity the substituents on the glucose ring are omitted.

FIG. 9. Intramolecular hydrogen bonds in the most stable conformers of the cis- and trans-isomers of compounds 3 and 4 as determined by DFT (B3LYP/6-31+G(d,p)) calculations in water.

FIG. 10. Types of Beta-turns according to the Hutchison-Thornton (1994) classification, and an illustration of an exemplary β-turn

FIG. 11. Structure of exemplary spirocyclic glucose-3(S)-hydroxyproline hybrids (Glc3 (S)HypHs).

FIG. 12. Potential explanation for the hydrolysis of prolyl amide bond in B.

FIG. 13. Selected ¹H-¹H ROESY cross-peaks in CD₂Cl₂ (2-11, 2-12 and 2-16) or H₂O (2-13) as well as potential hydrogen bonds from temperature coefficient experiments in DMSO-d₆ for major isomer in peptides 2-11, 2-12 and 2-16 and in H₂O/D₂O (9/1) for major isomer in 2-13.

FIG. 14. An exemplary glucose-templated proline-lysine chimera (GlaProLysC).

FIG. 15. Assignment of streochemistry at anomeric carbon in compounds 3-4 and 3-5 through 1D nOe experiments (recorded in CDCl₃).

FIG. 16. A possible explanation for stereoselective reductive amination using Felkin model.

FIG. 17. Assignment of stereochemistry at C-2′ position in compound 3-15 through 1D nOe experiment (recorded in C₆D₆). Some of the substituents in the glucose ring are omitted for clarity.

FIG. 18. Assignment of stereochemistry at C-5′-position in compounds 3-13 and 3-14 through 1D nOe experiment (recorded in C₆D₆).

FIG. 19. Assignment of cis and trans isomers in compound 3-23 in D₂O using 1D nOe experiments. The same experiments were used to assign the cis/trans isomers in compounds 3-22, 3-24 and 3-25.

FIG. 20. The n→π* interaction (looking down C^{α}—N bond).

FIG. 21. Pyrrolidine conformation in compounds 3-24 and 3-25.

FIG. 22. Suggested intramolecular H-bonds based on temperature coefficient experiments for compounds 3-24 and 3-25.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Intramolecular Hydrogen Bond-Controlled Prolyl Amide Isomerization in Glucosyl 3′(S)-hydroxy-5′-hydroxymethyl proline Hybrids—the Influence of a C-5′-hydroxymethyl Substituent on the Thermodynamics and Kinetics of Prolyl Amide Cis/Trans Isomerization

### Introduction

Proline (Pro) is the only cyclic amino acid of the twenty DNA-encoded amino acids, which is characterized by limited rotation of the 0 dihedral angle (fixed at ˜−75° as its side chain is fused to the peptide backbone. As a result there is a reduction in the energy difference between the prolyl amide cis- and trans-isomers making them nearly isoenergetic; this leads to a higher cis N-terminal amide content relative to the other amino acids. The kinetics of the prolyl cis/trans isomerization reaction is the rate-determining step in the folding pathways of many peptides and proteins (Brandits et al., 1975; Schmid and Baldwin, 1978; Hurle et al., 1990; Jackson and Fersht, 1991). Moreover, proline induces β-turns and extended helical structures (polyproline helix) in peptides that are crucial in protein/protein and protein/peptide interactions (Stryer, 2005). In nature, proline undergoes post-translational modifications such as hydroxylation to 4(R)-hydroxyproline (4-Hyp) and 3(S)-hydroxyproline (3-Hyp) (Reddy et al., 2004; Buku et al., 1980; Nakajima and Volcani, 1969; Taylor et al., 1969; taylor et al., 1994). Hydroxylation of proline is critical to the thermal stability and modulation of the local stability of the triple helix in collagens (Berg and Prockop, 1973; Vitagliano et al., 2001; Holmgren et al., 1998; Bretscher et al., 2001; Jenkins and raines, 2002) and contributes to the stability of the poly-Hyp helix in plant-derived Hyp-rich glycopeptides (Pearce and Ryan, 2003.

Over the years a plethora of proline analogs such as C^{β}-, C^{γ}- and C^{δ}-substituted prolines (Beausoleil and Lubell, 1996; Delaney and Madison, 1982; Samanen et al., 1990; Quancard et al., 2004), azaprolines (Che and Marshall, 2004), pseudoprolines (Tam and Miao, 1999), silaproline (Cavelier et al., 2002), proline-amino acid chimera (Sharma and Lubell, 1996), fused bicyclic proline (Jeannotte and Lubell, 2004; Wagaw et al., 1997; Kuwano et al., 2000; Viswanathan et al., 2003; Koep et al., 2003) and fused glucose-proline analogs (Owens et al., 2007) have been developed to study the structural and biological properties of proline surrogates in peptides (Cluzeau and Lubell, 2005; Blankley et al., 1987; Dumy et al., 1997; Li and Moeller, 1996). In particular, pseudoprolines bearing two substituents adjacent to the endocyclic nitrogen of proline and C^{δ}-substituted prolines containing bulky substitutents have been shown to increase the prolyl amide cis conformer ratio in peptides and peptide mimics (Beausoleil and Lubell, 1996; Dumy et al., 1997; Keller et al., 1998). Incorporation of pseudoprolines into peptides has been shown to induce a “kink” conformation in the peptide backbone, originating in the preference for cis amide bond formation. This prevents peptide aggregation, self association and β-structure formation thus improving the solvation and coupling kinetics of the growing peptide chain considerably (Wöhr et al., 1996).

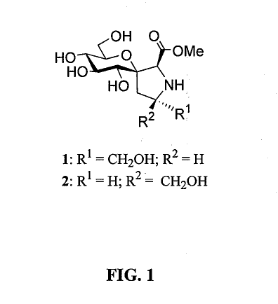

Recently, the inventors have reported on the synthesis of spirocyclic glucose-3(S)-hydroxy-5′-hydroxymethyl)proline hybrids (Glc3′(S)-5′(CH₂OH)HypHs) 1 and 2 (FIG. 1) (Zhang and Schweizer, 2005). Compounds 1 and 2 exhibit several intriguing features. The spirocyclic nature of the gluco-derived scaffold constrains the pyrrolidine ring of proline and introduces artificial post-translational modifications (hydroxylation+glycosylation). Chemical manipulations and derivatizations of the glucose-derived polyol scaffold provide an opportunity to tailor the chemical, physical and pharmacodynamic properties of Glc3′(S)-5′(CH₂OH)HypHs-containing peptides (Gruner et al., 2002). Moreover, compounds 1 and 2 contain a hydroxymethyl substituent adjacent to the imino function of proline which may permit control of prolyl amide cis/trans isomerization via hydrogen bonding, electrostatic or steric interactions. It is noteworthy that the influence of C-5′-substituted proline analogs capable of forming polar interactions on the thermodynamics and kinetics of prolyl amide cis/trans isomerization has not yet been investigated.

### Results

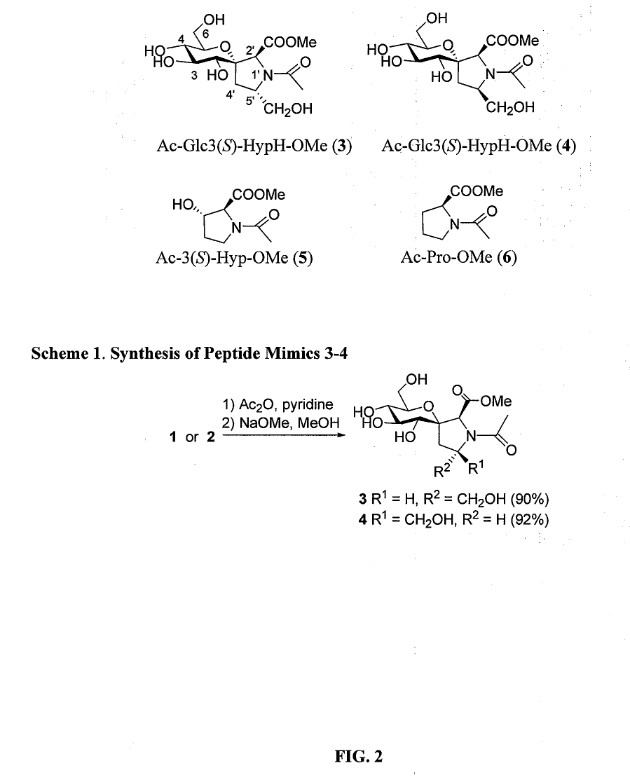

Herein, the inventors describe the thermodynamics and kinetics of prolyl N-terminal amide isomerization of peptide mimics 3 and 4 (FIG. 2). Compounds 5 and 6 (Jenkins et al., 2003) serve as reference compounds and were selected to study how 3(S)-hydroxylation of proline influences the kinetics and thermodynamics of prolyl amide cis/trans isomerization. The inventors initially selected C-terminal methyl esters to avoid complications arising from competing intramolecular hydrogen bonding of C-terminal amides (Cox and Lectka, 1998; Mizushima et al., 1952; Liang et al., 1992). Furthermore, the amide bond order of 3-6 can be assessed by FT-IR without interference with C-terminal amide. Subsequently, the inventors extended the study to C-terminal methylamides.

Synthesis of peptide mimics 3-6. Peptide mimics 3 and 4 were synthesized by acetylation of proline analogs 1 and 2 in pyridine and acetic anhydride followed by O-deacetylation using a solution of sodium methoxide in methanol (Scheme 1) (FIG. 2). Peptide mimic 5, was synthesized according to a modified procedure (Jenkins et al., 2003) while peptide mimic 6 was purchased.

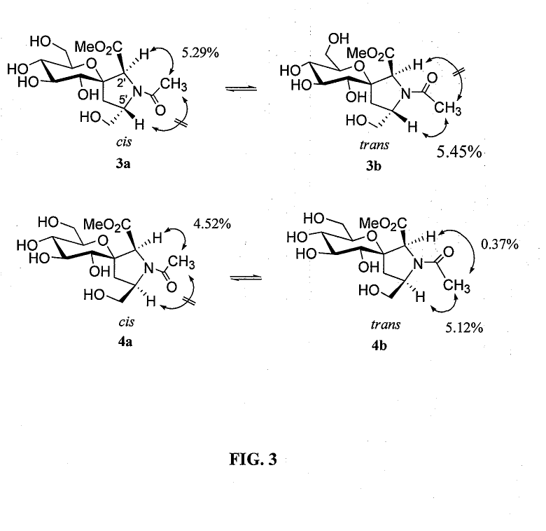

Assignments of N-terminal geometry for both major and minor isomers of 3 and 4 and determination of K_{t/c}. Identification of prolyl amide trans and cis isomers were based on multiple one-dimensional GOESY experiments in CD₃OD in which the optimized resolution was obtained (FIG. 3). For instance, subjection of H-2′ signal in prolyl amide cis isomer 3a to a one-dimensional GOESY experiment showed interproton effect to the N-terminal methyl group (5.2% nOe relative to the H-2′ signal). By comparison, no interproton effect was observed between H-5′ and the N-terminal methyl group. The same diagnostic tools were used to assign the prolyl amide isomers in compounds 3b, 4a and 4b and the observed interproton effects are summarized in FIG. 3.

The inventors also observed that the ¹³C NMR chemical shifts of the C-2′ atoms of the trans rotamers in compounds 3 and 4 are high-field shifted (0.8-1.6 ppm) relative to the cis isomers irrespective of the solvent used (Table 1). For instance, in methanol, the inventors observed C-2′ for cis isomer 3a at 72.80 ppm while the trans isomer 3b appeared at 71.74 ppm in the ¹³C-NMR. This result is consistent with previous findings by Beausoleil and Lubell (1996) and may serve as an empirical rule to assign the prolyl amide cis and trans isomers in cases where GOESY experiments cannot be performed.

TABLE 1
Chemical Shift of C-2′ in Cis and Trans Isomers of 3 and 4

| Compds | | 3^{a} | 4^{a} | 3^{b} | 4^{b} |
|---|---|---|---|---|---|
| C-2′ | cis | 73.63 | 73.55 | 72.80 | 72.67 |
| (ppm) | trans | 72.66 | 72.30 | 71.74 | 71.08 |

^{a}measured in D₂O;
^{b}measured in CD₃OD.

For each compound 3-6, the ratio of trans/cis isomers was calculated by integrating and averaging as many well-resolved proton signals as possible in the ¹H-NMR spectra (Table 2) (taylor et al., 2003). The inventors found that the hydroxymethyl substitutent at C-5′ enables tuning of the prolyl amide cis/trans ratio. For instance, compound 4 shows a slight preference for the cis rotamer (53%) while its C-5′ epimer exists predominantly as the trans rotamer (77%). In comparison, reference compounds 5 and 6 exhibit nearly identical cis/trans ratios confirming that the presence of an electron withdrawing hydroxyl group in 5 has no measurable effect on the prolyl amide cis/trans rotamer population (Jenkins et al., 2003).

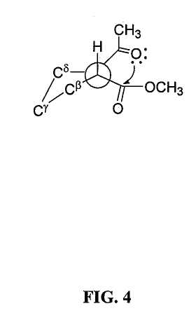

In order to compare the results with other reported proline analogues, the inventors converted peptide esters 3 and 4 into N′-methylamides 7 and 8 by nucleophilic displacement (Supporting Information). Peptide mimics 7 and 8 show a significantly increased cis rotamer population when compared to esters 3 and 4. Similar observations have been made by others and these results have been explained by enhanced n→π* interactions (Derider et al., 2002; Hinderaker and Raines, 2003; Hodges and Raines, 2006) of the oxygen lone pair of the (i−1) trans amide residue to the antibonding orbital of the C═O bond belonging to the Pro (i) residue (FIG. 4) (Taylor et al., 2003). The fact that an amide carbon is less electron deficient (Hinderaker and Raines, 2003) than an ester carbon has been used to explain higher trans ratios in prolyl amide of C-terminal esters when compared to prolyl amide of C-terminal amides (Taylor et al., 2003).

TABLE 2
Trans/cis ratio^{[a]} K_{t/c} (±0.04) and (cis
% ± 3%) Isomer of 3-6 in Water

| Compd. | 3 | 4 | 5 | 6 |
|---|---|---|---|---|
| k_{t/c} (cis %) | 3.35 (23%) | 0.88 (53%) | 4.88 (17%) | 6.14 (14%) |

^{[a]}Determined by 500 MHz NMR at 25 °C.

The cis population of peptide mimics 7 and 8 are presented in Table 3 together with previously published data for proline analog 9 (Beausoleil and Lubell, 1996), 5-methylproline analogs 10 and 11 (Delaney and Madison, 1982) and 5-tent-butylproline analogs 12 and 13 (Beausoleil and Lubell, 1996). These results show that 8 induces a high cis rotamer population (74±3%) while 7 exhibits a reduced cis rotamer population (38±3%). Interestingly, the stereochemistry at C-5′ of 7 and 8 seems to have a reverse effect on the equilibrium constant of isomerization by comparison with tert-butyl proline analogues 12 and 13 (Table 3).

Kinetics of cis-trans prolyl amide bond isomerization in peptide mimics 3-6. The kinetics of cis/trans isomerization for compounds 3-6 were determined by ¹H-NMR spectroscopy inversion-magnetization transfer experiments (Perrin and Dwyer, 1990; Reimer et al., 1998) in D₂O (Table 4). Because the rates for cis/trans isomerization are extremely slow in protic solvents, the inventors performed these experiments at elevated temperatures (Stein, 1993). At 83° C., the trans-to-cis rate constants of isomerizations (k_{tc}) follows the order 4<5≈6<3. A 200-fold rate difference is observed for diastereomeric amides 3 and 4. In comparison, hydroxyprolyl amide 5 and prolyl amide 6 exhibit nearly identical rate constants indicating that the 3(S)-hydroxy group has little effect on the kinetics of isomerization.

TABLE 3
Cis Population of Prolyl N-acetyl N′-methylamides in D₂O

| Compd. | 7 | 8 | 9^{a} | 10^{b} | 11^{b} | 12^{a} | 13^{a} |
|---|---|---|---|---|---|---|---|
| Cis(±3%) | 38 | 74 | 27 | 25 | 30 | 49 | 66 |

^{a}taken from Ref. 9;
^{b}taken from Ref. 31.

TABLE 4
Rate Constants of Prolyl Amide Isomerization for 3-6

| Amide | Kct^{[a]} (s⁻¹) | k_{tc}^{[b]}(s⁻¹) |
|---|---|---|
| 3 | 0.18 ± 0.01 | 0.08 ± 0.004 |
| 4 | 16.19 ± 1.19 | 19.82 ± 1.45 |
| 5 | 2.63 ± 0.28 | 1.02 ± 0.09 |
| 6 | 2.95 ± 0.12 | 0.82 ± 0.04 |

^{[a]}Carried out in D₂O at 83 °C.
^{[b]}Calculated from k_{ct} and K_{t/c};

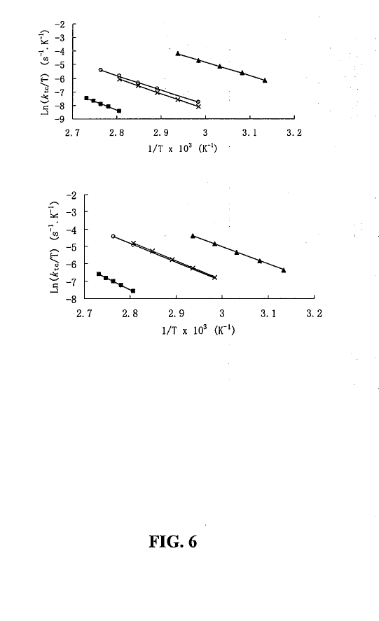

The effects of temperature on k_{ct} and k_{tc} were analyzed by Eyring plots (FIG. 6) (Eyring, 1935). The values for ΔH^{‡} and ΔS^{‡} (Table 5) were calculated from linear least squares fits of the data in these plots (Eyring, 1935). The activation parameters demonstrate that the free energy barriers to isomerization of compounds 3-6 are enthalpic in origin. Interestingly, amide 3 exhibits a significantly increased activation enthalpy (3.9-6.8 kcal/mol) when compared to compounds 4-6. However, the activation enthalpy is partially compensated by a higher activation entropy.

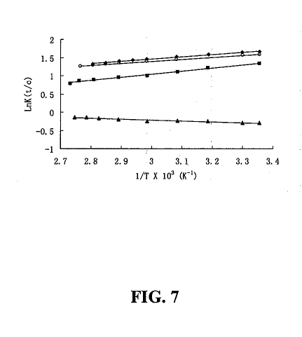

Thermodynamics The effects of temperature on the values K_{t/c}=(k_{ct}/k_{tc}) for each compound were measured directly by NMR spectroscopy over the temperature range 25-93° C. The resulting data were analyzed by Van't Hoff plots (FIG. 7). Amides 3, 5 and 6 have a positive slope indicating that the major trans isomer becomes increasingly favored as the temperature decreases. However, compound 4 displays a negative slope and shows a reduction in the magnitude of K_{t/c}. In this case the major cis isomer becomes increasingly favored as the temperature decreases. Values for ΔH^{o} and ΔS^{o} were calculated from linear least-squares fits of these plots (Table 6).

TABLE 5
Activation Enthalpies (ΔH‡) and Entropies (ΔS‡) as
Derived from Eyring Plots in D₂O for 3-6. Additionally
the Free Energies of Activation at 298K (ΔG‡) are given.

| | cis to trans^{[a]} | | | trans to cis^{[a]} | | |
|---|---|---|---|---|---|---|
| | ΔH‡^{[b]} | ΔS‡^{[c]} | ΔG‡^{[d]} | ΔH‡^{[b]} | ΔS‡^{[c]} | ΔG‡^{[d]} |
| 3 | 26.1 | 11.1 | 22.8 | 26.4 | 10.2 | 23.4 |
| 4 | 19.8 | 2.2 | 19.1 | 19.6 | 1.9 | 19.0 |
| 5 | 21.6 | 3.6 | 20.5 | 21.5 | 1.6 | 21.1 |
| 6 | 21.9 | 4.8 | 20.5 | 22.5 | 3.9 | 21.4 |

^{[a]}Error limits obtained from the residuals of the linear least squares fitting of the data to equation, ln (k/T) = (−ΔH‡/R)(1/T) + ΔS‡/R + ln(k_{B}/h), were 1-3% for ΔH‡ in compounds 3-6, and 3-7% for ΔS‡ in 3 and 6, and 16-26% for ΔS‡ in 4 and 19-48% for ΔS‡ in 5;
^{[b]}unit: kcal/mol.
^{[c]}unit: cal/mol.K.
^{[d]}unit: kcal/mol.

FT-IR analysis of amide-I band (C═O stretching) for 3-6 in D₂O. The inventors also measured the frequency of the amide I vibrational mode, which results primarily from the C═O stretching vibration (Jackson and Mantsch, 1995). The traditional picture of the amide resonance predicts that an increase in C═O bond order is accompanied by a decrease in C—N bond order. Such a decrease in C—N bond order would facilitate cisitrans isomerization of the amide bond. In D₂O, the amide I vibrational modes of 6, 3, 5 and 4 are at 1608, 1609, 1612 and 1613 cm⁻¹, respectively and follow the order 6˜3<5˜4. It has been shown that changes in the free energy of activation (ΔG^{‡}) for prolyl peptide bond isomerization are proportional to changes in the frequency (v) of the amide I vibrational mode (Eberhardt et al., 1996). The results demonstrate that 3-Hyp-based amide 5 is blue shifted (Δv=4 cm⁻¹) when compared to prolyl amide 6. However, these subtle differences are not detectable by the kinetic assay. A similar trend is observed for spirocyclic amides 3 and 4. Obviously, the blue shift (Δv=4 cm⁻¹) observed for 4 is too small to account for the significant changes in ΔG^{‡} between 3 and 4 and this suggests that other factors unrelated to inductive effect and C═O bond order are the cause for this dramatic rate difference (Eberhardt et al., 1996).

TABLE 6
Thermodynamic Parameters for Isomerization 3-6

| | ΔH°^{[a]} | ΔS°^{[a]} | ΔG°^{[b]} |
|---|---|---|---|
| Amide | (kcal/mol) | (cal/mol•K) | (298K) |
| 3 | −1.67 ± 0.06 | −2.93 ± 0.20 | −0.80 ± 0.13 |
| 4 | 0.50 ± 0.06 | 1.05 ± 0.16 | 0.19 ± 0.11 |
| 5 | −1.09 ± 0.03 | −0.48 ± 0.08 | −0.95 ± 0.05 |
| 6 | −1.21 ± 0.04 | −0.71 ± 0.12 | −1.01 ± 0.08 |

^{[a]}Error limits obtained by linear least-squares fitting the data of the Van't Hoff plots to equation ln K_{tc} = (−ΔH°/R)(l/T) + ΔS°/R;^{b]}Carried out in D₂O; ±SE determined by integration of two or more sets of trans/cis isomers.

Temperature Coefficient (Δδ/ΔT) Measurement of OH Resonances for 3 and 4 in DMSO-d₆. The inventors next considered the effect of hydrogen bonding on prolyl amide cis/trans isomerization. The temperature coefficients (Δδ/ΔT) provide information about intramolecular hydrogen bonding (Leeflang et al., 1992; St-Jacques et al., 1976). Previous studies have shown that (Δδ/ΔT)>−3.0 ppb/deg are a diagnostic tool for the detection of intramolecular H-bonding (Leeflang et al., 1992; St-Jacques et al., 1976). The 1D spectra of compounds 3 and 4 recorded between 20 to 45° C. in 5-deg steps in DMSO-d₆ were analyzed and the temperature coefficients were determined (Table 7). The data show that the temperature coefficients for HO-6′ exhibits the highest value of all hydroxyl groups. The temperature coefficients follow the general order OH-2<OH-3<OH-4<OH-6<OH-6′. In particular, the low (Δδ/ΔT) value for the cis isomers in 3 (−3.94 ppb/K) and 4 (−3.75 ppb/K), respectively, suggests the presence of intramolecular H-bonding involving OH-6′. In the trans isomers of 3 and 4, the Δδ/ΔT of HO-6′ were reduced to −4.13 and −4.50 ppb/K, respectively. The relatively small value of HO-6′ in both trans isomers, of 3 and 4 indicates that it also could be involved in hydrogen bonding for both compounds.

TABLE 7
Temperature Coefficient (Δδ/ΔT, ppb/K)
for Compounds 3 and 4 in DMSO-d₆

| | | HO-2 | HO-3 | HO-4 | HO-6 | HO-6′ |
|---|---|---|---|---|---|---|
| 3 | cis | −6.87 | −6.49 | −5.85 | −5.29 | −3.94 |
| | trans | −6.98 | −6.94 | −5.67 | −5.14 | −4.13 |
| 4 | cis | −7.58 | −6.94 | −5.85 | −5.48 | −3.75 |
| | trans | −6.94 | −6.57 | −5.55 | −4.92 | −4.50 |

Conformational analysis of the pyrrolidine ring in 3 and 4 using Density Functional Theory (DFT). In order to gain insight into the conformational properties of peptide mimics 3 and 4 in solution and to study how intramolecular hydrogen-bonding influences the kinetics and thermodynamics of prolyl amide isomerization in compounds 3 and 4, the inventors performed DFT calculations. On the basis of previous experience and literature reviews (Koch and Holthausen, 2000; Cramer, 2004), the inventors selected the B3LYP level of theory (Becke, 1993; Lee et al., 1988; Stephens et al., 1994), which has been shown good enough to provide accurate predictions of molecular structures, and the 6-31+G(d, p) basis set (Hehre, 2003), which is a relatively large basis set augmented by diffuse and polarization functions to account for correlation effects. Additionally, solvent effects were taken into account using Tomasi's Polarized Continuum Model (PCM) (Tomasi et al., 2005). In this model, the solvent is represented as a polarizable medium characterized by its dielectric constant (i.e. water has a dielectric constant of 78.4 at 25° C. and 1 atm), and the solute molecules are placed in a cavity within the solvent.

A multi-step procedure was used to determine the structures of 3 and 4 to ensure that the calculations covered the entire conformational space. First, the conformational space was searched using the MMFF (molecular mechanics) force field and a Monte-Carlo search procedure, which was augmented by systematically varying the initial starting structure. After the resulting conformers where superimposed to remove duplicates, 443 and 457 unique structures, respectively, for compounds 3 and 4 were found at the MMFF level. These conformers were used as input for gas-phase DFT optimizations, which were subsequently followed by solvation optimizations. The final Gibbs free energies were calculated and compared to determine the (Boltzmann) distribution of cis and trans isomers, which are summarized in Table 8. Full details on the computational protocol can be found in the supporting information.

TABLE 8
The Calculated Distribution (%) of Cis and
Trans Conformers for 3 and 4 in D₂O^{a}

| Compd. | calculated | experimental |
|---|---|---|
| 3 (cis) | 29.15 | 23 ± 3% |
| 3 (trans) | 70.85 | 77 ± 3% |
| 4 (cis) | 57.75 | 53 ± 3% |
| 4 (trans) | 42.25 | 47 ± 3% |

^{a}Calculations at the B3LYP/B3LYP/6-31 + G(d, p)/PCM level of theory

The calculated distribution of prolyl amide cis/trans isomers in compounds 3 and 4 is in good agreement with the experimental data determined by ¹H-NMR integration (Table 8). For compound 4, the cis isomer population was calculated to be 57.7%, while the experimentally determined value is 53±3%. A slightly weaker agreement is observed for 3. In this case, the calculated cis ratio is 29%, while the experimental value is 23±3%.

TABLE 9
Range of Backbone and Endocyclic Torsion Angles⁴⁵ (°) for the Most Stable Conformers of 3 and 4
Accounting for 99.5% of the Total Conformer Population Determined by DFT Calculations.
For clarity the substituents on the pyran ring are omitted.

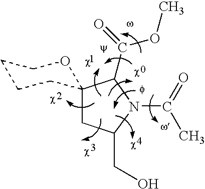

| | | ω | φ | ψ | ω | χ⁰ | χ¹ | χ² | χ³ | χ⁴ |
|---|---|---|---|---|---|---|---|---|---|---|
| 3 | cis | −11, −1° | −71, −62° | 153, 154° | 177 ± 1° | −15, −22° | 30, 34° | −35, −33° | 21, 25 | −5, 0° |
| | | | −28, −27 | | −177 ± 1° | | | | | |
| | trans | 165, 178° | −67, −53° | 132, 154° | 178 ± 1° | −16, −6° | 25, 30° | −36, −34° | 24, 30° | −14, -7° |
| | | | | −30, −27° | −179 ± 1° | | | | | |
| 4 | cis | 3, 12° | −91, −77° | 152, 154° | 177 ± 1° | −21, −15 | 31, 33 | −37, −32° | 20, 27° | −8, 0° |
| | | | | −28, −27° | −177 ± 1° | | | | | |
| | trans | −172, −169° | −86, −79° | 152, 154° | 178 ± 1° | −21, −19° | 30, 33° | −33, −30° | 18, 21° | −1, 2° |
| | | | | −28, −26° | −179 ± 1° | | | | | |

In terms of structure, the computational data show that the pyranose ring in 3 and 4 exists in a ⁴C₁ chair conformation consistent with the diaxial coupling constants J_{2,3}, J_{3,4}, J_{4,5}≧8.8 Hz observed in the ¹H-NMR. The range of peptide backbone (ψ′, φ, ψ and ω) and endocyclic torsion angles (χ⁰, χ¹, χ², χ³ and χ⁴) for peptide mimics 3 and 4 are displayed in Table 9. Full details for all conformers can be found in the supporting information. With the exception of the ψ torsion angle that exists in two families at ψ˜153° and ψ˜−28°, all other torsion angles show preference for only one narrowly defined range. Values close to 0° for the ψ′ torsion angels define the cis prolyl amide isomer, while values close to ±180° describe the trans isomer.

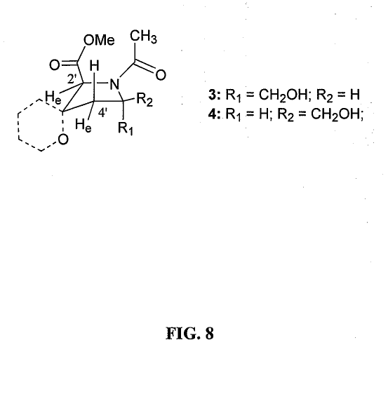

The observed small χ⁴ torsion angle (−14°≦ψ⁴≦0°) for the conformers of compounds 3 and 4 indicate a preferred C^{β}-exo pucker in which the basal plane is defined by C^{γ}, C^{δ}, N and C^{α} (FIG. 8). Similar puckerings of the pyrrolidine ring have been observed in the crystal structure of 3-Hyp-containing peptide mimics and have also been proposed in 3-Hyp-containing collagenous peptide sequences (Jenkins et al., 2003). The relative close value for χ⁴ and χ⁰ in the trans rotamer of 3 indicates a twisted conformation between a C^{β}-exo and a C^{γ}-endo pucker. The C^{β}-exo conformation places the endocyclic oxygen substituent in an axial position as observed for trans 3(S)-hydroxyproline-containing dipeptides (Taylor et al., 2005). In this conformation the pyrrolidine ring will be stabilized by gauche interaction and a stabilizing σ(C^{γ}—H)→σ*(C^{β}—O) interaction. This conformation is further supported by characteristic long range “W” coupling constants (J˜1.0 Hz) between H-2′ and H-4′_{eq} in both isomers of 3 and 4.

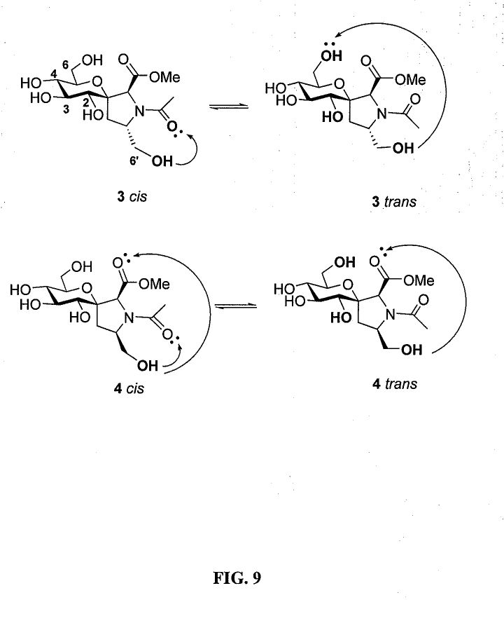

The most stable conformers accounting for 99.5% of the total conformer population in compounds 3 and 4 were analyzed for the presence and absence of characteristic hydrogen bonds. The inventors used a ≦2.5 Å bond length cutoff between the hydrogen atom in the donor and the acceptor atom to ensure that only strong hydrogen bonds were selected (Morozov et al., 2004). The inventors found two types of hydrogen bonds in the dominant rotamer populations of 3 and 4 (FIG. 9). For compound 3, the first type of hydrogen bond exists between OH-6′ and OH-6 (6′-OH—-OH-6) with an average bond distance of around 1.9 Å. This hydrogen bond is only observed in the trans rotamers of 3. The second type of hydrogen bond exists between OH-6′ and the N-terminal carbonyl (6′-OH—O═C—N). This hydrogen bond has an average bond length of 1.8 Å and is only found in the cis rotamers. In addition, a third hydrogen bond with a bond distance of 1.9 Å (not shown in FIG. 9) is found between OH-2 and the C-terminal carbonyl (2-OH—-O═C′—C-2′) in one out of the 43 most stable trans conformers.

For compound 4, the first type of hydrogen bond exists between 6′-OH and the N-terminal carbonyl (6′-OH—O═C′—N). This H-bond has an average distance of 1.8 Å and is found only in the cis isomer. A second type of H-bond with a bond length of 1.9 Å exists between 6′-OH and the C-terminal carbonyl (C^{δ}—CH₂—OH—O═C′—C-2′) that is found in both cis and trans conformers (FIG. 9).

### Discussion

The pyrrolidine ring in proline exhibits two predominant pucker modes: C-4 (C^{γ}) exo and endo envelope conformers. In the case of unsubstituted proline, the endo puckering mode is favored over the exo mode (Koskinen et al., 2005). However, the puckering propensity can be controlled by proper choice of ring substituents (Koskinen et al., 2005). For example, previous studies have shown that introduction of electronegative substituents like 4(R)-hydroxy- or 4(R)fluoro-substituents results in the stabilization of the C^{γ}-exo conformation and the trans prolyl amide isomer (Eberhardt et al., 1996; Renner et al., 2001).

The results confirm that naturally occurring 3(S)-hydroxyproline does not lead to a measurable increase in the trans prolyl amide isomer population (Jenkins et al., 2003) when compared to unsubstituted proline. For the first time the inventors studied the effect of 3(S) hydroxylation on the kinetics of prolyl amide cis/trans isomerization. The nearly identical rate constants observed for peptide mimics 5 and 6 at elevated temperature demonstrate that 3(S)-hydroxylation has little effect on the kinetics of prolyl amide cis/trans isomerization. A similar effect was observed for 4(R)—OH proline (Eberhardt et al., 1996; renner et al., 2001). Raines et al. have studied the crystal structure of Ac-3(S)-Hyp-OMe 5 and concluded it to be intermediate between a ¹E envelope and ¹E₂ twisted conformation (Jenkins et al., 2003). In the envelope conformation the flap atom is C^{β}(C-3′). In the twisted conformation, atoms N, C^{β} and C^{δ} (C-5′) form the basal plane. Atom C^{β} resides 0.456±0.004 Å above that plane, and C^{δ} resides 0.153±0.005 Å below that plane (Jenkins et al., 2003). Taken together, these results suggest that the hydroxyl group at the β- or γ-position in proline affects the puckering of the pyrrolidine ring in the model peptides without influencing the kinetics and thermodynamics of prolyl amide cis/trans isomerization.

These findings are in contrast to the results obtained with Glc3′(S)-5′(CH₂OH)HypHs-containing peptide mimics 3 and 4. Compounds 3 and 4 demonstrate that kinetics and thermodynamics of cis/trans isomerization are greatly affected by the presence of polar groups either by hybridization with D-glucose or incorporation of a hydroxymethyl substituent into the C-5′ (δ-)-position of proline. The stereochemistry of the hydroxymethyl substituent at the 5′-position influences both the rate of cis/trans isomerization and the stability of the cis/trans isomers.

Compared to proline and 3(S)Hyp-containing peptide mimics 5 and 6, both Glc3′(S)-5′(CH₂OH)HypHs-modified peptide mimics 3 and 4 display a higher cis isomer population. The cis isomer population is greatly enhanced in mimic 4. Substitution of the C-terminal methyl ester in compounds 3 and 4 by a methylamide group increases the cis isomer ratio further as expected by enhanced n→π* donation. Indeed, methylamides 7 and 8 exhibit 38% and 74% cis isomer, respectively. Comparison of the temperature coefficients for all hydroxyl groups indicates that the increased cis prolyl amide isomer ratio in peptide mimics 3, 4, 7 and 8 is hydrogen bond-mediated and involves the hydroxymethyl group located at the C-5′ position of proline.

Additional evidence for the involvement of intramolecular hydrogen bonds in the stabilization of the prolyl amide cis isomer is provided by detailed DFT conformational searches. Analysis of the most stable prolyl amide cis conformers in 3 and 4 indicate the presence of a strong hydrogen bond between 6′-OH and the carbonyl group of NAc (6′-OH—O═C—N). The higher cis isomer ratio in 4 is due to the formation of a second hydrogen bond involving 6′-OH and the carbonyl of the carboxymethyl group. Alternatively, the trans prolyl amide bond in 3 may be stabilized by the formation of a hydrogen bond between 6′-OH and 6-OH. In addition, the calculations support the notion that the H-bond between 6′-OH O═C—N is stronger in compound 4 when compared to 3. For instance, for compound 4 ten out of the twenty most stable conformers possess this type of H-bond, while the same H-bond was not present in the most stable 20 conformers of compound 3.

Conformational analysis of the pyrrolidine ring in the highly populated conformers in peptide mimics 3 and 4 indicates that the pucker ressembles a C^{β}-exo conformation in both compounds, which is very similar to the conformation previously observed in the crystal structure of 5. This conformation places the endocyclic oxygen substituent in an axial position as observed for trans 3(S)-hydroxyproline-containing dipeptides (Taylor et al., 2005). In this conformation, the pyrrolidine ring will be stabilized by gauche interaction between endocyclic nitrogen and endocyclic oxygen and a stabilizing σ(C^{γ}—H)→σ*(C^{β}—O) interaction. A similar C^{β}-exo conformation was also observed in the more lipophilic silaproline (Sip) analogue (Cavelier et al., 2002). However, in this case, no increase of the cis isomer population was noted. It is noteworthy that the calculated C^{β}-exo conformation is closely related to the C^{γ}-endo pucker that is found in most proline-containing peptides, the polyproline helix and the collagen triple helix.

Quite unexpected are the results for the kinetics of prolyl amide cis/trans isomerization in compounds 3 and 4. Peptide mimic 3 exhibits an unusually high activation barrier when compared to epimer 4. For instance, an approximately 200-fold difference in k_{tc} and a 90-fold rate difference in k_{ct} are observed between compounds 3 and 4. In contrast, nearly identical rate constants are observed for parent compounds 5 and 6. Interestingly, while cis/trans isomerization is kinetically inhibited in compound 3, compound 4 accelerates isomerization relative to parent compounds 5 and 6. For instance, compound 4 exhibits approximately a 6-fold rate acceleration for k_{ct} and a 20-fold acceleration for k_{tc} when compared to the parent compounds. Comparison of the activation enthalpies and activation entropies of compounds 3-6 indicate that enthalpic changes are responsible for these rate differences. Enthalpic changes could be the result of ground- and transition-state stabilization or destabilization (Fisher et al., 1994).

Previous studies by Lubell have shown that introduction of methyl substituents at the β-position in proline and 4-hydroxyproline induced ψ dihedral angles around 150°. This places the C-terminal carbonyl oxygen in a position which disfavors amide pyramidalization by Coulomb interactions in the transition state (Beausoleil et al., 1998). DFT calculations on both compounds 3 and 4 indicate a ψ dihedral angle around 153° and ˜−28° in their most stable conformers which demonstrates that a similar destabilization may exist in peptide mimics 3 and 4. However, compounds 3 and 4 exhibit dramatic rate differences.

The inventors suggest that the presence or absence of intramolecular H-bonds involving 6′-OH, the endocyclic nitrogen and the carboxymethyl group are responsible for the observed rate differences. Computational models on the most stable (ground-state) conformers of 4 suggest that the hydrogen bond 6′-OH—O═C—OMe places the 6′-OH in an orientation to interact with the lone pair of the pyrimidalized nitrogen in the transition state. By comparison, the absence of this hydrogen bond in 3 prevents stabilization of the transition state. Moreover, the H-bond 6′-OH—HO-6 observed in the trans isomer of 3 places the 6′-OH in a geometry that prevents H-bonding to the endocyclic nitrogen in the transition state thereby leading to a higher activation energy in 3 (Fisher et al., 1994).

Comparison of the activation enthalpies (ΔH^{‡}) for cis-to-trans isomerization and trans-to-cis isomerization of compounds 3-6 indicate that the kinetics of cis/trans isomerization is enthalpically driven. The lower (ΔH^{‡}) observed for compound 4 when compared to 5 and 6 could be the result of a developing hydrogen bond between the hydroxymethyl substituent at C-5′ and the lone pair of the pyramidalized nitrogen in the transition state. In contrast, the higher activation enthalpy observed for peptide mimic 3 when compared to reference compounds 5 and 6 could be the result of increased Coulomb repulsion induced by the ψ dihedral angle around 153° (Beausoleil et al., 1998; Halab and Lubell, 1999).

Previously, accelerated cis/trans isomerizations have been observed in model peptides containing 8-tert-butyl-proline (Halab and Lubell, 1999) or pseudo-proline (ΨPro) (Keller et al., 1998). In these cases, a twisted amide bond caused by the steric strain of the bulky substituents adjacent to the endocyclic imide results in a destabilized ground state (Keller et al., 1998; Halab and Lubell, 1999). In addition, the increased distance between C-5′ and the hetero atom such as sulfur or oxygen leads to a shortened 5′-C—N bond in pseudo-prolines (Keller et al., 1998). As a consequence, dimethyl groups at the C-5′ position come closer to the isomerizing bond and may stabilize the transition state by the presence of the hydrophobic alkyl group (Albers et al., 1990). The results indicate that insertion of a polar group(s) at the 6-position of proline or in the form of sugar-proline hybrids provides an alternative route to control the kinetics of prolyl amide cis/trans isomerization.

### Conclusions

The inventors have studied the thermodynamic and kinetic properties of a series of polyhydroxylated Glc3′(S)-5′(CH₂OH)-HypHs-containing peptide mimics. The study shows that the insertion of polar substituents capable of forming hydrogen bonds in the C-5′ position of proline greatly impacts the kinetics and thermodynamics of prolyl amide cis/trans isomerization. DFT calculations and chemical shift temperature coefficient measurements indicate that these changes are not due to conformational changes in the pucker of the pyrrolidine ring, but rather are the result of intramolecular hydrogen bonding in water.

Computational modeling of the pyrroldine ring indicates that the pyrrolidine ring preferes a C^{β}-exo pucker. However, close inspection of the C^{β}-exo pucker suggests that it is closely related to the C^{γ}-endo pucker that occurs frequently in proline-containing peptides and proteins. The preferable adoption of the prolyl amide cis conformation in glucose-3′(S)-hydroxy-5′(R)-hydroxymethyl proline 4 allows its use as selective cis-XAA-GlcProH bond inducer to chemically introduce constraint into peptides and proteins and to test the cis-imide bond as a structural requirement for the bioactive conformation. Moreover, the presence of the unprotected glucose moiety in GlcProH provides opportunities to explore the effect of glycosylation in unusual glycopeptides while decoration of the gluco-based polyol scaffold provides rich opportunities to tailor the physical, chemical, hydrophobic, lipophilic nucleophilic, and pharmacodynamic properties of proline mimetics and proline-containing peptidomimetics.

### Experimental Section

(1S)-2,3,4,6-Tetrahydroxy-1′-N-acetyl-5′(S)-hydroxymethylene-spiro[1,5-anhydro-D-glucitol-1,3′-L-proline methyl ester] (3) The compound 1 (30 mg, 0.10 mmol) was dissolved in a mixture of pyridine and acetic anhydride (1 mL, 1:1) and stirred for 12 hours at room temperature. After that, the pyridine and acetic anhydride were removed in vacuo. The crude product was dissolved in methanol (1 mL) followed by addition of sodium methoxide (22 mg, 0.39 mmol) and stirred for 3 hours. The solution was stirred with Amberlite IRC-50S ion-exchange resin (H⁺) for 15 minutes. The mixture was filtered and filtrate was concentrated and purified by the flash column chromatography (ethyl acetate/methanol: 4/1) to get compound 3 as a colorless oil (30 mg, 90%) [α]_{D}=31.4 (c 1.00, MeOH); ¹H NMR (500 MHz, D₂O): δ=1.81 (s, cis, 0.53H), 2.09 (s, trans, 2.47H), 2.11-2.34 (m, both rotamers, 2H), 3.27-3.35 (m, 2H), 3.39-3.46 (m, both rotamers, 1H), 3.50-3.66 (m, 6H), 3.67-3.72 (dd, 1H, J=12.6 Hz, J=2.5 Hz), 3.74-3.79 (dd, trans, 0.83H, J=11.1 Hz, J=5.3 Hz), 3.82-3.86 (dd, cis, 0.17H, J=11.11 Hz, J=5.3 Hz), 4.17-4.24 (m, both rotamers, 1H), 4.26 (s, trans, 0.81H), 4.43 (s, cis, 0.19H); ¹³C NMR (75 MHz, D₂O): trans-rotamer, δ=25.1, 25.7, 53.4, 60.2, 61.0, 63.2, 69.6, 69.9, 70.5, 74.1, 75.0, 86.0, 171.4, 174.8; cis-rotamer, 22.0, 24.6, 53.9, 59.7, 62.2, 63.2, 69.6, 69.9, 71.3, 74.1, 75.0, 87.5, 171.9, 174.6; HRMS calcd for C₁₄H₂₄NO₉ [M+H]⁺ 350.1451, found 350.1462.

(1S)-2,3,4,6-Tetrahydroxy-1′-N-acetyl-5′(R)-hydroxymethylene-spiro[1,5-anhydro-D-glucitol-1,3′-L-proline methyl ester] (4) The compound 2 (35 mg, 0.11 mmol) was dissolved in a mixture of pyridine and acetic anhydride (1 mL, 1:1) and stirred for 12 hours at room temperature. After that, the pyridine and acetic anhydride were removed in vacuo. The crude product was dissolved in methanol (1 mL) followed by addition of sodium methoxide (25 mg, 0.46 mmol) and stirred for 3 hours. The solution was stirred with Amberlite IRC-50S ion-exchange resin (H⁺) for 15 minutes. The mixture was filtered and filtrate was concentrated and purified by the flash column chromatography (ethyl acetate/methanol: 4/1) to get compound 4 as a colorless oil (37 mg, 92%) [α]_{D}=63.9 (c 1.00, MeOH); ¹H NMR (500 MHz, D₂O): δ=1.86 (s, cis, 1.56H), 1.94 (dd, cis, 0.52H, J=14.3 Hz, J=10.3 Hz), 1.99-2.08 (m, 1.92H), 2.37 (dd, cis, 0.52H, J=14.3 Hz, J=7.2 Hz), 2.48 (dd, trans, 0.48H, J=14.3 Hz, J=7.2 Hz), 3.25-3.35 (m, 2H), 3.38-3.45 (m, 1H), 3.53-3.75 (m, 7H), 3.80-3.88 (m, 1H), 4.04 (m, cis, 0.52H), 4.14 (m, trans, 0.48H), 4.37 (s, cis, 0.52H), 4.40 (s, trans, 0.48H); ¹³C NMR (75 MHz, D₂O): trans-rotamer, 21.3, 29.1, 53.6, 58.8, 61.1, 63.9, 69.7, 69.9, 70.2, 74.1, 75.3, 85.0, 172.0, 174.9; cis-rotamer, 21.6, 27.0, 53.7, 59.0, 61.1, 62.9, 69.8, 70.0, 71.2, 74.2, 75.4, 86.1, 172.0, 174.9; HRMS calcd for C₁₄H₂₄NO₉ [M+H]⁺, 350.1451, found 350.1456.

N-acetyl-3(S)-hydroxy-L-proline methyl ester (5). To a solution of Boc-3(S)-OH-Pro-OH (100 mg, 0.43 mmol), which was purchased from ACS Synthesis Company in U.S.A. and methyl iodide (0.08 mL, 1.29 mmol) in N,N-dimethylformamide was added cesium carbonate (154 mg, 0.47 mmol) and stirred for a hour at 0° C. The reaction was quenched with water (3 mL) and extracted with ethyl acetate (3×10 mL). The combined organic layers were concentrated to afford the methyl ester, which was treated with a mixture of trifluoroacetic acid and dichloromethane (v/v, 1 mL/1 mL) and stirred for 2 hours at room temperature. The mixture was concentrated at vacuo to afford the intermediate NH₂-TFA-3(S)-OH-Pro-OMe. This salt was dissolved in methanol (2 mL) and treated with triethylamine (0.12 mL, 0.86 mmol) and acetic anhydride (0.12 mL, 1.29 mmol) for 12 hours at room temperature. The mixture was concentrated and purified by flash chromatography (ethyl acetate/methanol: 20/1) to get the compound 5 (78 mg, yield 96%). ¹H NMR (500 MHz, D₂O): δ=1.82-2.10 (both isomers, m, 5H, N-amide methyl group and γ-protons), 3.50 (cis, dd, 0.43H, δ-protons, J=5.78 Hz, J=9.40 Hz), 3.60-3.66 (trans, m, 3.95H, methyl group of ester and δ-protons), 3.68 (cis, methyl group of ester, 0.63H), 4.24 (s, trans, 0.79H, α-proton), 4.41 (m, trans, 0.78H, β-proton), 4.47 (s, cis, 0.21H, α-proton), 4.55 (m, cis, 0.22H, β-proton); ¹³C NMR (75 MHz, D₂O): trans-rotamer, δ=21.4, 32.4, 46.5, 53.6, 67.4, 73.4, 172.6, 174.0; cis-rotamer, δ=21.7, 30.7, 44.9, 53.9, 69.2, 74.8, 172.0, 174.2; MS (ES, [M+Na]); m/z calcd for C₈H₁₃NNaO₄ 210.07, found 210.13.

N-acetyl-L-proline methyl ester (6). This compound is purchased from Bachem and used without further purification. ¹H NMR (500 MHz, D₂O): δ=1.65-2.27 (both isomers, m, 7H, N-amide methyl group, β and γ-protons), 3.37 (cis, m, 0.26H, δ-protons), 3.52 (trans, m, 1.74H, δ-protons), 3.58-3.70 (both isomers, partially overlapping, methyl group of ester, 3H), 4.31 (m, trans, 0.8714, α-proton), 4.57 (m, cis, 0.13H, α-proton); ¹³C NMR (75 MHz, D₂O): trans-rotamer, δ=21.6, 24.6, 29.6, 48.8, 53.3, 59.4, 173.3, 175.4; cis-rotamer, S=22.8, 24.6, 31.0, 47.0, 53.6, 61.1, 173.7, 175.0; MS (ES, [M+Na]⁺); m/z calcd for C₈H₁₃NNaO₃ 194.08, found 194.18.

(1S)-2,3,4,6-Tetrahydroxy-1′-N-acetyl-5′(S)-hydroxymethylene-spiro[1,5-anhydro-D-glucitol-1,3′-L-proline methylamide] (7). [α]_{D}=46 (c 0.8, MeOH); ¹H NMR (500 MHz, D₂O): δ=1.79 (s, cis, 1.15H), 2.06 (s, trans, 1.85H), 2.11-2.30 (m, both rotamers, 2H), 2.56 (s, trans, 1.83H), 2.61 (s, cis, 1.17H), 3.25-3.50 (m, 4.4H), 3.54-3.70 (m, 2.6H), 3.74 (dd, trans, 0.64H, J=5.1 Hz, J=11.2 Hz), 3.74 (dd, cis, 0.36H, J=4.7 Hz, J=10.7 Hz), 4.12-4.25 (m, 2H); ¹³C NMR (75 MHz, CD₃OD): cis rotamer, δ=22.6, 26.7, 26.8, 61.6, 62.9, 64.7, 71.4 (2 carbons), 74.3, 75.6, 77.3, 88.5, 171.7, 173.6; trans rotamer, S=22.3, 25.7, 26.7, 61.5, 62.7, 63.9, 71.4 (2 carbons), 73.4, 75.5, 77.2, 87.2, 171.5, 173.2; HRMS calcd for C₁₄H₂₅N₂O₈ [M+H]⁺ 349.1611, found 349.1626.

(1S)-2,3,4,6-tetrahydroxy-1′-N-acetyl-5′(R)-hydroxymethylene-spiro[1,5-anhydro-D-glucitol-1,3′-L-proline methylamide] (8). [α]_{D}=62.3 (c 0.6, MeOH); ¹H NMR (500 MHz, D₂O): δ=1.86 (s, cis, 2.22H), 2.05 (s, trans, 0.78H), 2.11 (m, cis, 0.74H), 2.25 (m, 1.26H), 2.56 (s, trans, 0.78H), 2.61 (s, cis, 2.22H), 3.25-3.35 (m, 2H), 3.37-3.43 (m, 1H), 3.53-3.61 (m, 2.79H), 3.64-3.70 (m, 1.21H), 3.96-4.04 (m, 1H), 4.07 (s, cis, 0.74H), 4.09-4.18 (m, 1H), 4.19 (s, trans, 0.26H); ¹³C NMR (75 MHz, CD₃OD): cis rotamer, δ=23.1, 26.0, 26.4, 59.6, 60.7, 62.7, 71.1, 71.3, 74.5, 75.6, 77.4, 87.0, 172.9, 173.8; trans rotamer, S=21.9, 28.4, 26.3, 59.3, 62.3, 63.1, 71.1, 71.6, 73.3, 75.9, 77.4, 85.5, 173.2, 173.8; HRMS calcd for C₁₄H₂₅N₂O₈ [M+H]⁺ 349.1611, found 349.1618.

Thermodynamics. The equilibrium constants for the interconversion of the cis and trans isomers of 3-6 were determined by measuring the peak area of the 1H resonance for the two isomers. Peak areas were measured with the program Spinworks 2.5. Experiments were conducted at 298-360 K. Equilibrium constants (K_{t/c}=trans/cis ratios) were calculated directly from the peak areas.

Inversion-magnetization transfer NMR experiments were performed on Bruker AMX500 spectrometer equipped with selective excitation units, and ¹H and broadband heteronuclear probes. Samples of 3-6 were prepared at a concentration of 0.01 M in D₂O. The rate of prolyl peptide bond isomerization can not be detected by this method at room temperature. Experiments were therefore conducted at elevated temperature at 356 K. Temperature settings of the spectrometer were calibrated to within 1° C. by reference to a glycol standard.

FT-IR spectroscopy. Samples of 3-6 were prepared at concentration of 0.10 M in D₂O. FTIR spectra were recorded on a Nicolet 5PC spectrometer. Experiments were performed at 25° C. using CaF₂ in a Spectra Tech circle cell. The frequency of amide I vibrational modes was determined to within 2 cm⁻¹.

Temperature coefficient (Δδ/ΔT) experiment: 1 D ¹H-NMR spectroscopy of 17 mM solutions of 19 and 20 in 100.0% Me₂SO-d₆ were recorded on a Bruker AMX500 at 20° C., and from 20 to 45° C. with increments of 5° C., using routine techniques. Chemical shift (6) are expressed in ppm and calibrated with respect to the residual DMSO signal (1H, 2.49 ppm).

Supporting Information Available: The ¹H and ¹³C NMR spectra of 3-8; 1 D NOE experiments of compounds 3, 4, 7 and 8. The HSQC NMR spectra of 3-6. Plots of intensity versus mixing time for the magnetization transfer experiments on 3-6; Table of kinetic and thermodynamic data; The FT-IR spectrum of the amide-I stretch region of 3-6; The temperature coefficient experiments for 3 and 4; Full computational details and characterization of cis/trans isomers. This material is available free of charge via the Internet at http://pubs.acs.org.

### Example 2

### Influence of Glucose-Templated Proline Hybrids on the β-Turn Conformation of the Peptide Fragment Ac-Leu-D-Phe-Pro-Val-NMe₂ of Gramicidine S

### Introduction

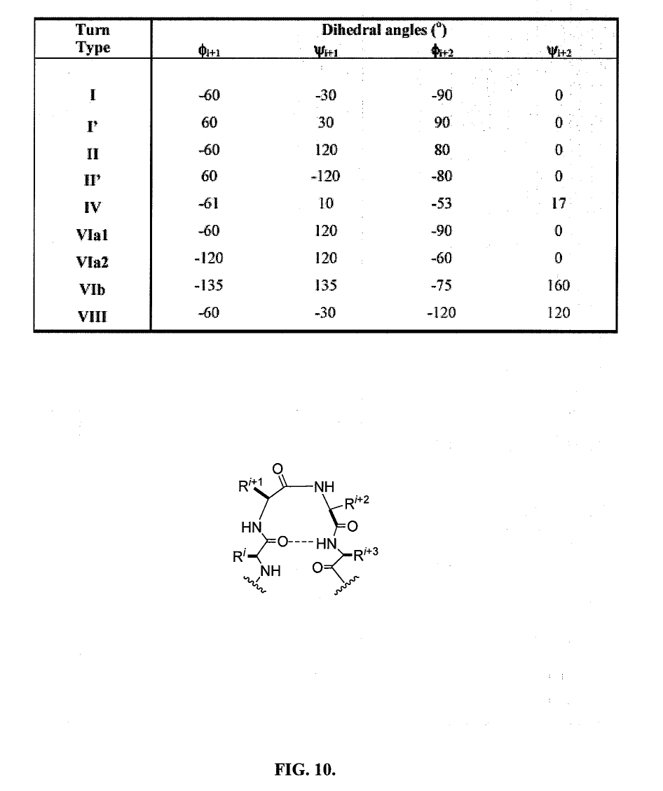

β-turns play an important role in the folding process of proteins and peptides (Richardson, 1981; Wilmot and Thornton, 1988; rose et al., 1985) and are often involved in molecular recognition processes (Creighton, 1992; Rizo and Gierasch, 1992). β-turns consist of four consecutive residues in a non-helical region in which the polypeptide chain folds back on itself by nearly 180 degrees (Lewis et al., 1971). β-turns are often stabilized by an intramolecular hydrogen-bond between the carbonyl oxygen of the first residue (i) and the amide proton of the fourth one (i+3) (FIG. 10). Many β-turn mimetics have been developed to study the structural and biological properties of peptides. In particular, the use of proline analogues is an attractive strategy in this respect. Due to its conformational restrictions imposed by the pyrrolidine ring, the proteinogenic amino acid proline has an exceptional tendency to act as a turn inducer to generate reverse turn structures like β-turns and β-hairpins in peptides and proteins (Branden and Tooze, 1991; Stryer, 1999). In particular, the cis geometry of the proline N-terminal amide bond induces the type VI β-turn in which proline residue situates at the i+2 position of the peptide bend.

Although type VI β-turn is a relatively rare secondary structure, it plays important roles in protein folding (Fisher, 1994; Liu et al., 1991). In addition, type VI β-turn have been found in some important recognition events of bioactive proteins. For example, a type VI β-turn conformation has been proposed for thrombin-catalyzed cleavage of the V₃ loop of HIV gp120, a prerequisite to viral infection (Johnson et al., 1994). Over the years many proline analogues have been synthesized and used for tuning the peptidyl-prolyl cis-trans isomerization in peptides and proteins (Dugave and Demange, 2003). For instance, (2S,5R)-5-tert-butylproline favors predominantly a cis-conformation due to steric hindrance in short peptide models that adopt a VI β-turn (Halab and Lubell, 2002). Another sterically hindered residue, the δ,δ-dimethylproline, has been developed as a substitute to lock proline in the cis conformation in tripeptides (An et al., 1999). Azaprolines (AzPro) in which the α-carbon is replaced by a nitrogen favor the cis isomer conformation of the azaproline-preceding amide bond by electronic effects (Didierjean et al., 1997; Zouikri et al., 1998). Pseudoproline ΩPro, containing an oxozolidine or thiazolidine ring, exhibit very high prolyl amide cis ratios (Dumy et al., 1997; Kern et al., 1997; Keller et al., 1998).

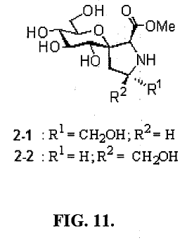

Recently, the inventors have reported the synthesis of spirocyclic glucose-3-hydroxyproline hybrids Glc3(S)HypHs 2-1 and 2-2 (FIG. 11) (Zhang and Schweizer, 2005). These proline analogues exhibit several features. The spirocyclic nature of the gluco-derived scaffold constrains the pyrrolidine ring of proline and introduces artificial post-translational modifications (hydroxylation+glycosylation).

Chemical manipulations and derivatizations of the glucose-derived polyol scaffold provide an opportunity to tailor the chemical, physical and pharmacodynamic properties of Glc3(S)HypHs-containing peptides as previously shown by the inventors (Owens et al., 2007). The peptide mimics, Ac-Glc(3S)-Hyp-OMe and Ac-Glc(3S)-Hyp-NHMe display interesting conformational properties. For example, when compared to Ac-Pro-OMe or Ac-(3S)-Hyp-OMe, (5′R)-Glc(3S)-Hyp favored the prolyl amide cis rotamer (53˜75%) with an accelerated cis/trans isomerization in water. Whereas the diastereomer (5′S)-Glc(3S)-Hyp favors the trans rotamer (62˜77%) with a retarded cis/trans isomerization in water. Based on the previous results, the inventors were interested in exploring the conformational role of these proline analogues in model peptides. The inventors selected the tetrapeptide Ac-Leu-D-Phe-Pro-Val-NMe₂ that forms the β-turn portion of Gramicidine S (GS) as a model (Jennotte and Lubell, 2004). In this peptide proline occupies the i+2 position of a β-turn. The goal was to study how substitution of the proline residue by Glc3(S)HypHs influences the conformational and β-turn inducing properties.

### Results and Discussion

Synthesis of MOM-protected Glc3(S)HypHs. The use of unprotected polyhydroxylated amino acids in peptide chemistry often results in low coupling yields and difficult purification due to their nucleophilic hydroxyl groups. To avoid this potential complication the inventors decided to use methoxymethyl (MOM) groups as temporary hydroxy protection (Walker et al., 2006). The MOM protecting group is relatively small, does not deactivate adjacent nucleophiles and can easily removed under acidic conditions (Walker et al., 2006; Sicheral and Wittmann, 2005). The building block 2-5 was prepared from 2-1 through a three-step procedure (Scheme 2).

Initially, the free imine 2-1 was treated with benzyl chloroformate (Cbz-Cl) and sodium carbonate to provide the Cbz-protected carbamate followed by the introduction of MOM groups using N,N-diisopropylethylamine and chloromethyl methyl ether to provide MOM-protected intermediate 2-3 in good yield (Walker et al., 2006). Subsequently, lithium hydroxide-based hydrolysis of the methylester in dioxane produced the desired acid 2-5 in 88% yield. Surprisingly, only a small portion of methyl ester (˜10%) was epimerized at the α position. The stereochemistry at the α-position of acid 2-5 was confirmed by converting it into the corresponding methyl ester 2-3. Applying the same procedure to its diastereomer 2-2 resulted in extended epimerization (˜1:1) at the α-position. Fortunately, the mixture of the two acids was separated by column chromatography. The assignment of α-stereochemistry of compound 2-6 was based on the same chemical method as previously described for compound 2-5.

Synthesis of tetrapeptides 2-11 to 2-13. With MOM-protected building block 2-5 at hand, the synthesis of the tetrapeptide was carried out using solution-phase peptide chemistry (Scheme 3). Dipeptide 7 was prepared via coupling of NH₂-Val-NMe₂ to compound 2-5 (DIEA, TBTU, DMF, 80%). The N-terminal Cbz group was then removed by catalytic hydrogenolysis in quantitative yield using (Pd(OH)₂, H₂, MeOH) to provide the free imine 2-7 in high yield. Synthesis of tripeptide 2-9 proceeded from the dipeptide 2-7. Coupling with Fmoc-D-Phe-OH (DIEA, PyBOP, DMF, 82%) provided Fmoc-protected tripeptide, which was treated with a mixture of piperidine and N,N-dimethylformamide to produce the tripeptide 2-9 with an unprotected N-terminus in quantitative yield. Because of the steric hindrance of the N-terminus, the coupling reagent PyBOP was used to improve the yield (Frerot et al., 1991). The coupling of tripeptide 2-9 and Fmoc-Leu-OH was carried out by using TBTU as coupling reagent in DMF followed by a deprotection-acylation procedure to provided MOM-protected tetrapeptide 2-11 in 92% yield. The same synthetic procedure was applied to the synthesis of tetrapeptide 2-12. Similar yields were obtained in each step except for the coupling reaction between dipeptide 2-8 and Fmoc-D-Phe-OH. In this case, a low yield for tripeptide 2-10 was obtained and the dipeptide 2-8 was recovered in 45% yield. This might be due to the steric hindrance. Modifying the reaction conditions like increasing reaction time, amount of amino acid and coupling reagent did not improve the yield.

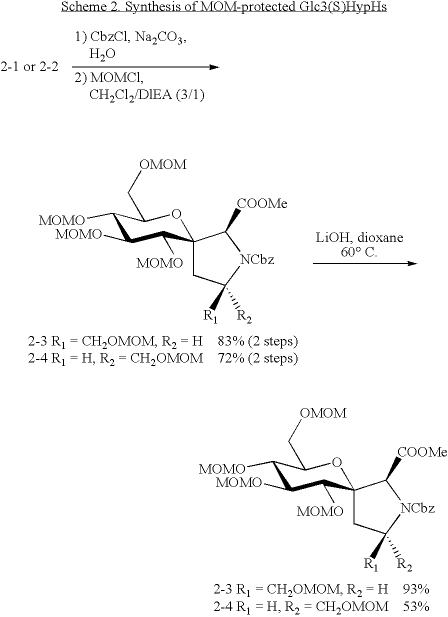

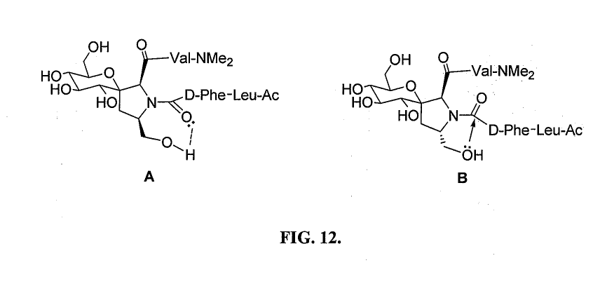

Finally, exposure of compound 2-11 to acidic conditions (0.3 N HCl in MeOH) resulted in complete deblocking of the MOM protecting groups and afforded unprotected tetrapeptide 2-13 (Scheme 3) in excellent yield (90%). Unfortunately, applying the same acidic conditions to tetrapeptide 2-12 resulted in an inseparable mixture of the dipeptides 2-14 and 2-15. Very likely the hydrolysis proceeds via anchimeric assistance of the hydroxymethyl substituent at the 5′-position of proline (FIG. 12).

The major isomer of prolyl amide in A is cis conformation (see the conformational analysis) in which the 5′-hydroxymethyl group may form a hydrogen bonding to the carbonyl oxygen of the prolyl amide. This restricts the 5′-hydroxymethyl group into a geometry in which the carbonyl carbon of the prolyl amide can not be nucleophilically attacked by the 5′-hydroxymethyl group using a Bürgi-Dunitz trajectory (Bürgi et al., 1974). Whereas the major trans isomer of the prolyl amide in B not only makes the prolyl amide carbon more accessible to the hydroxymethyl substituent at C-5′ but also facilitate nucleophilic attack on the electrophilic amide carbon, due to the absence of an intramolecular hydrogen bond between the prolyl amide oxygen and 5′-hydroxymethyl group.

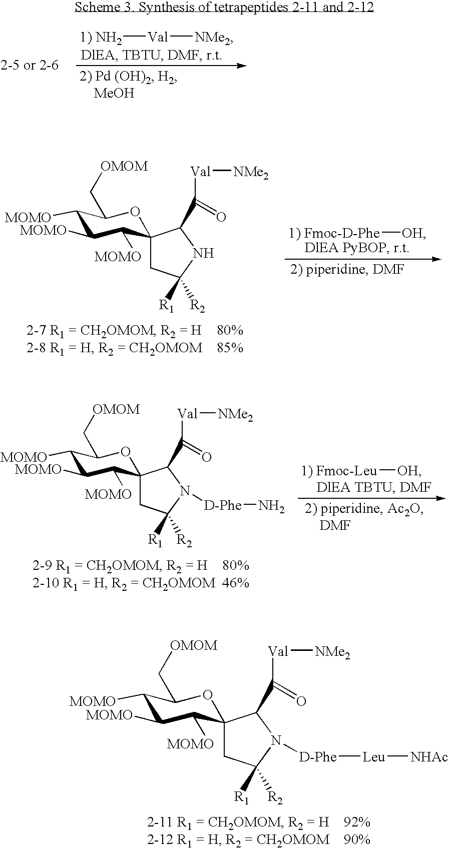

The successful synthesis of unprotected tetrapeptide 2-13 demonstrates that MOM protecting groups are compatible with solution phase peptide synthesis for (5′R)-Glc3(S)HypHs 2-1. Whereas the high acid lability of diastereoisomer (5′S)-Glc3(S)HypHs 2-2 does currently not permit use in peptide synthesis. For reference purposes the parent peptide 2-16 was also prepared by standard solution phase peptide chemistry (Jeannotte and Lubell, 2004).

Conformational analysis of tetrapeptides 2-11 to 2-13 and 2-16 using NMR. Although the reference peptide 2-16 aggregated in CD₂Cl₂ at ≧10 mM (Jeannotte and Lubell, 2004), no aggregation was observed for tetrapeptides 2-11 to 2-13 at concentrations ≦50 mM in CD₂Cl₂ or water. The lower tendency of aggregation of glycosylated peptides has previously been reported (runkel et al., 1998). The NMR spectra of all tetrapeptides were measured at the same concentration (8 mM). Intramolecular hydrogen bonding was evaluated by temperature coefficient experiments in DMSO-d₆ or H₂O/D₂O (9/1). Because exchangeable protons engaged in intramolecular hydrogen bonds are typically not influenced by strong hydrogen-bonding solvents (Kopple et al., 1969). The prolyl N-terminal geometry and turn structure were investigated by ROESY experiments (Misicka et al., 1998). The chemical shifts and coupling constants of the amide protons in tetrapeptides 2-11 to 2-13 and 2-16 are provided in Table 10.

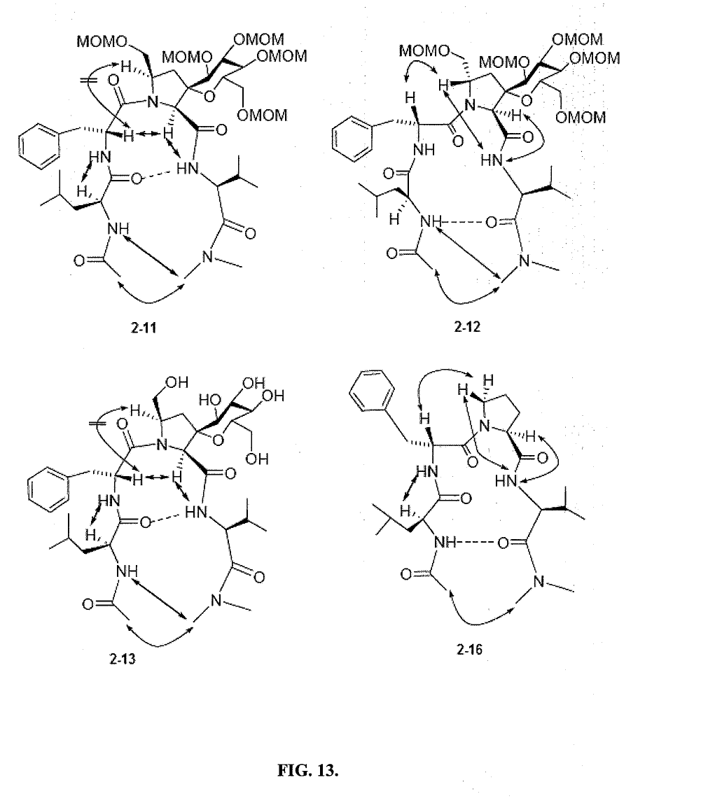

The tetrapeptide 2-11 containing MOM-protected (5′R)-Glc3(S)HypHs. The proline mimetic in tetrapeptide 2-11 coexists as prolyl amide cis (72%) and trans (28%) in CD₂Cl₂, The major prolyl amide cis isomer is confirmed by a long range ROESY correlation between C^{α}H_{D-Phe} and C^{α}H_{(5′R)-Glc3(S)HypHs}. The presence of a turn conformation in 2-11 is supported by long range ROESY correlations between the Leu-NH or the N-terminal acetyl group and the C-terminal dimethyl amide singlets (FIG. 13). In addition, sequential ROESY correlations were observed between the neighboring C^{α}H_{Leu} and NH_{Phe} as well as the C^{α}H_{(5′R)-Glc3(S)HypHs} and NH_{Val} indicating their proximity in the majorconformer.

The chemical shifts and coupling constants of the amide protons in 2-11 (Table 10) indicate that the cis conformer has a different folding structure when compared to reference peptide 2-16 in CD₂Cl₂. The downfield chemical shift (δ=8.20 ppm) and large temperature coefficient value for Val-NH (Table 11) support that the Val-NH is involved in an intramolecular hydrogen bonding. Therefore, a type VI β-turn conformation is suggested for the prolyl amide cis conformer of 2-11 in which the type VI β-turn is stabilized by a 10-membered ring hydrogen bond between (C═O)_{Leu} at i position and NH_{Val} at i+3 position (Fisher and Angew, 1994; Liu et al., 1991). This hydrogen bond is further supported by the observation that the amide proton of Val undergoes little chemical shift changes (Δδ=0.08 ppm) when dissolved in DMSO and CD₂Cl₂.

Due to the low concentration of trans conformer of 2-11 in CD₂Cl₂ (72% cis), a full conformational analysis of the minor prolyl amide trans isomer was not possible. However, the similar chemical shifts of the amide protons between the trans isomer of 2-11 and the trans isomer of 2-16 supports these amide protons have a similar chemical environment in CD₂Cl₂. Furthermore, the temperature coefficient experiments (Table 11 and 12) support that the NH_{Leu} of both trans conformers of 2-11 and 2-16 were involved in a hydrogen bonding to (C═O)_{Val}.

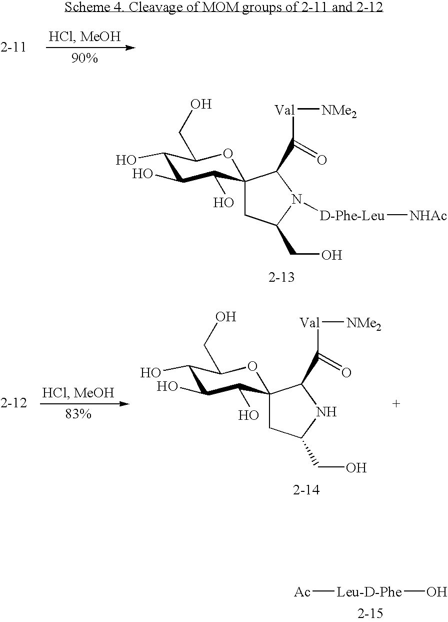

The tetrapeptide 2-12 containing MOM-protected (5′S)-Glc3(S)HypHs. The NMR spectrum showed one major isomer (93%) in CD₂Cl₂. The ROESY correlation (FIG. 13) between CH_{D-Phe} and C^{δ}H_{(5′R)-Glc3(S)HypHs} confirmed its trans geometry. The presence of a turn conformation in 2-12 is supported by long range ROESY correlations between the Leu-NH or the N-terminal acetyl group and the C-terminal dimethyl amide singlets (FIG. 13). Temperature coefficient experiments in DMSO-d₆ (Table 12) indicate that NH_{Leu} is involved in hydrogen bonding to the carbonyl group of valine. In addition, the similar chemical shifts observed for the amide protons among compounds 2-12, 2-16 and GS (Table 10) suggests that the β-turn conformation of 2-12 resembles the β-turn conformation that occurs in GS.

Interestingly, the prolyl amide cis conformer of 2-12 may form a type VI β-turn structure on the basis of chemical shift analysis and hydrogen bond pattern of amide protons relative to the cis conformer of 2-11 (Table 10). That is, the stereochemistry at 5′-position (8 position) of Glc3(S)HypHs can be used to control the equilibrium constants of cis/trans isomerization (72% cis for 2-11, 93% trans for 2-12) without disturbing the β-turn conformation of each isomer (a type VI β-turn for cis conformer; original Alum for trans conformer).

The tetrapeptide 2-13 containing (5′R)-Glc3(S)HypHs. The increased polarity of unprotected tetrapeptide 2-13 made it possible to investigate its conformation by NMR in water. ROESY experiments on compound 2-13 demonstrate that the major isomer exhibits a prolyl amide cis conformation (91%) in H₂O/D₂O (9/1). This assignment is supported by a ROESY correlation between C^{α}H_{D-Phe} and C^{α}H_{(5′R)-Glc3(S)HypHs} (FIG. 13). In comparison with MOM-protected peptide 2-11 the increased cis population in 2-13 may be due to the hydrogen bonding between (C═O)_{D-Phe} and 5′-CH₂OH_{(5′R)-Glc3(S)HypHs}. The long range ROESY correlations between the Leu-NH or the N-terminal acetyl group and the C-terminal dimethyl amide singlets indicated the presence of a turn conformation in 2-13 (FIG. 13). Temperature coefficient experiments (Table 13) indicate that compound 2-13 exists in a type VI β-turn conformation that is stabilized by an intramolecular hydrogen bond between the NH_{Val} and (C═O)_{Leu} in its prolyl amide cis conformer.

According to amide temperature coefficient experiments (Δδ/ΔT<−7.45 ppb/K, Table 13) it appears that the minor prolyl amide trans isomer of tetrapeptide 2-13 is not involved in hydrogen bonding. Due to the low concentration of the prolyl amide trans isomer it was not possible to observe ROESY correlations between the Leu-NH or the N-terminal acetyl group and the C-terminal dimethyl amide singlets.

Attempts to investigate the influence of the hydroxyl groups of the carbohydrate scaffold on the peptide backbone conformation failed due to poor resolution of the hydroxy protons in DMSO-d₆ (Chakraborty et al., 2004; Leeflang et al., 1992). As indicated above, (5′R)-Glc3(S)HypHs can efficiently increase the cis population of prolyl N-terminus that induces the type VI β-turn. Derivatization of the hydroxyl groups as MOM does not affect prolyl amide cis/trans isomerization as well as type VI β-turn conformation in tetrapeptide 2-11. In comparison, the MOM-protected diastereoisomer (5′S)-Glc3(S)HypHs stabilizes the prolyl amide trans conformer resulting in a conserved β-turn structure as observed in reference peptide 2-16. In conclusion, proline analogues 2-11 to 2-14 can be used to tune the hydrophilic or hydrophobic properties of peptides without disturbing their major bioactive conformation.

Solvent effects. In addition, the influence of different solvents on the geometry of prolyl N-terminus was studied (Table 14). The results show that equilibrium constant of MOM-protected 2-11 is not affected by the solvent polarity and ability of the solvent to donate a hydrogen bond. The unprotected tetrapeptide 2-13 also showed the absence of a strong solvent effect. However, an increased trans population was observed in aprotic DMSO when compared to CD₃OD and D₂O. In contrast, peptide 2-12 and reference peptide 2-16 display a solvent effect. In this case the prolyl amide trans rotamer population is greatly enhanced in nonpolar aprotic solvents and decreases with an increase in solvent polarity.

TABLE 10
Chemical shift (δ) for amide protons of tetrapeptides 2-11, 2-12, 2-13,
2-16 and Gramicidin S in CD₂C1₂, DMSO-d₆ and H₂O/D₂O (9/1)

| | | Leu-NH | PheNH | Val-NH |
|---|---|---|---|---|
| peptides | Solvent | δ (³J_{αH, NH}) | δ (³J_{αH, NH}) | δ (³J_{αH, NH}) |
| 2-11 | CD₂Cl₂ | 6.23(8.5) | 6.40(8.3) | 8.20(8.3) |
| (cis)^{a} | DMSO-d₆ | 7.65(9.0) | 8.18(9.7) | 8.12(7.5) |
| 2-11 | CD₂Cl₂ | 6.18(8.5) | 6.61(7.7) | 7.03(ND) |
| (trans)^{a} | DMSO-d₆ | 7.78(8.9) | 8.37(8.6) | 7.69(8.2) |
| 2-12 | CD₂Cl₂ | ND^{c} | ND^{c} | ND^{c} |
| (cis) | DMSO-d₆ | 7.65(9.0) | 8.34(8.7) | 7.45(7.p) |
| 2-12 | CD₂Cl₂ | 6.88(7.7) | 7.31(7.2) | 6.78(8.2) |
| (trans) | DMSO-d₆ | 7.79(8.5) | 8.27(8.9) | 7.51(8.1) |
| 2-13 | H₂O/D₂O | 7.98(6.6) | 8.25(7.7) | 9.17(8.1) |
| (cis) | DMSO-d₆ | 7.75(8.8) | 8.04(8.8) | 8.72(7.3) |
| 2-13 | H₂O/D₂O | ND^{c} | ND^{c} | ND^{c} |
| (trans) | DMSO-d₆ | 7.80(9.5) | 8.33(8.1) | 8.39(5.9) |
| 2-16 | CD₂Cl₂ | ND^{c} | ND^{c} | NDc |
| (cis) | DMSO-d₆ | 7.74(8.3) | 8.43(8.3) | 8.12(8.1) |
| 2-16 | CD₂Cl₂ | 7.76(7.5) | 7.12(5.5) | 6.85(7.0) |
| (trans) | DMSO-d₆ | 7.79(8.2) | 8.37(7.8) | 7.78(8.2) |
| Gramicidin | DMSO-d₆ | 8.32(9.2) | 9.05(2.6) | 7.21(9.7) |
| S (trans)^{b} | | | | |

^{a}Cis and trans are referred to as prolyl amide rotamers;
^{b}referred to lit. 15 and its supporting information;
^{c}ND = not determined

TABLE 11
Temperature coefficient values (Δδ/ΔT,
ppb/K) for peptides 2-11 in DMSO-d₆

| | Leu-NH | Phe-NH | Val-NH |
|---|---|---|---|
| cis | −6.00 | −8.13 | −3.75 |
| trans | −4.370 | −11.47 | −9.40 |

TABLE 12
Temperature coefficient values (Δδ/ΔT, ppb/K)
for peptides 2-12 and 2-16 in DMSO-d₆

| | | Leu-NH | Phe-NH | Val-NH |
|---|---|---|---|---|
| 2-12 | cis | −5.20 | −4.80 | −4.15 |
| | trans | −3.90 | −7.40 | −8.00 |
| 2-16 | cis | −4.00 | −8.00 | −8.00 |
| | trans | −3.92 | −7.60 | −6.20 |

TABLE 13
Temperature coefficient values (Δδ/ΔT, ppb/K)
for peptides 2-13 and 2-16 in H₂O/D₂O (9/1)

| | Leu-NH | Phe-NH | Val-NH |
|---|---|---|---|
| cis | −7.23 | −8.45 | −4.02 |
| trans | −7.91 | −7.72 | −7.45 |

TABLE 14
The equilibrium constant (K_{t/c}) of 2-11,
2-12, 2-13 and 2-16 in various solvents

| Compd. | CD₂C1₂ | CD₃OD | DMSO-d₆ | D₂O |
|---|---|---|---|---|
| 2-11 | 0.39 | 0.33 | 0.30 | —^{[b]} |
| 2-12 | 13.28 | 3.00 | 1.44 | —^{[b]} |
| 2-13 | —^{[b]} | 0.14 | 0.67 | 0.10 |
| 2-16 | 19.00 | 3.17 | 1.32 | —^{[b]} |

^{[a]}Determined by 500 MHz NMR at 25° C., error is ± 0.05%;
^{[b]}not soluble.

### Conclusions

MOM-protected Glc3(S)HypHs-based proline mimics have been successfully incorporated into target tetrapeptides using solution phase peptide chemistry. In particular, (5′R)-Glc3(S)HypH demonstrated its potential as a building block in peptide synthesis. Whereas the use of its diastereoisomer (5′S)-Glc3(S)HypH is limited due to accelerated hydrolysis of its N-terminal amide under acidic conditions.

As what the inventors expected, (5′R)-Glc3(S)HypH dramatically increases the cis population (91%) of prolyl amide in water. It is an efficient type VI β-turn inducer and can be used to explore structure activity relationships (SAR) of bioactive peptides in which the cis geometry of prolyl amide is required for their bioactivity. In addition, as observed in tetrapeptides 2-11 and 2-13, the derivatization of hydroxy groups can improve the solubility of tetrapeptides without affecting peptide folding (type VI β-turn). This suggests that Glc3(S)HypHs will find use in tuning the chemical, physical and pharmacodynamic properties of bioactive peptides without affecting their bioactive conformation.

In contrast, tetrapeptide 2-12 containing (5′S)-Glc3(S)HypH conserved a similar) β-turn conformation to the Gramicidin S-based peptide fragment Ac-Leu-D-Phe-Pro-Val-NMe₂ 2-16. Incorporation of 2-12 in GS may improve the antibacterial activity of GS. Moreover, replacement of Pro with Glc3(S)HypHs was found to reduce the aggregation of tetrapeptidea 2-11 to 2-13 relative to reference tetrapeptide 2-16. That is, incorporation of CTAA into peptides provides a strategy to improve the physical and chemical properties of peptides.

### Experimental

General ¹H and ¹³C NMR spectra were taken in CD₂Cl₂, CDCl₃, CD₃OD, D₂O at 500 MHz and 75 MHz, respectively. HRMS data were provided by the mass spectrometry laboratory in the department of chemistry at University of Alberta. The elemental analysis was performed by Guelph Chemical Laboratories LTD. Analytical thin layer chromatography was performed on 0.20 mm silica gel 60 Å plates. Flash chromatography was performed on 40-63 μm 60 Å silica gel.

(1S)-2,3,4,6-Tetra-O-methoxymethyl-1′-N-benzyloxycarbonyl-5′(R)-methyl methoxymethyl ether-spiro[1,5-anhydro-D-glucitol-1,3′-L-proline methyl ester] (2-3) To a mixture of compound 2-1 (80 mg, 0.26 mmol) and benzyl chloroformate (0.19 mL, 1.30 mmol) in water (2 mL) was added sodium carbonate (83 mg, 0.78 mmol). The reaction was stirred for 12 hours at room temperature and extracted with ethyl acetate (5×10 mL). The organic layers were collected, concentrated and purified by the flash column chromatography (ethyl acetate/methanol: 4/1) to get Cbz-propected intermediate (103 mg, 90%). Which was treated with 3 mL dichloromethane and cooled in an ice bath under nitrogen atmosphere, diisopropylethylamine (1 mL) was added dropwise, followed by a careful addition of chloromethyl methyl ether (0.71 mL, 9.36 mmol). A significant amount of white smoke formed in the reaction vessel. The reaction mixture was stirred in the dark for 48 hours during which the solution gradually turned red. After cooling to 0° C., saturated aqueous ammonium chloride (5 mL) was added. The contents diluted with water and extracted with dichloromethane (3×10 mL). The combined organic layers were dried (NaSO₄), filtered, and concentrated. The crude product was chromatographed on silical gel (from ethyl acetate/hexanes (1:1) to ethyl acetate) to afford the product 2-3 (128 mg, 83%). [α]_{D}=38.4 (c 1.4, CHCl₃); ¹H NMR (300 MHz, CDCl₃, two isomers): δ=2.08-2.21 (m, 1H), 2.44-2.56 (m, 1H), 3.22-3.44 (m, 15H), 3.48-3.77 (m, 10H), 4.00-4.15 (m, 1.36H), 4.27 (dd, 0.64H, J=8.9 Hz, J=3.9 Hz), 4.42 (s, 0.64H), 4.50-4.82 (m, 10.36H), 5.00 (d, 0.64H, J=13.0 Hz), 5.07-5.18 (m, 1.36H), 7.21-7.36 (m, 5H); ¹³C NMR (75 MHz, CDCl₃, two isomers): δ=31.5/32.9, 52.2/52.3, 55.0-56.5 (12 carbons), 66.7/66.8, 66.8/67.4, 69.4/70.2, 70.0/70.1, 72.1 (2 carbons), 74.8 (2 carbons), 76.4/76.6, 79.7/80.4, 84.8/85.4, 96.5-98.1 (10 carbons), 127.3-128.5 (10 aromatic carbons), 136.2/136.5 (aromatic carbons), 154.4/155.2, 169.9/170.1; HRMS (ES) calcd for C₃₀H₄₈NO₁₅ [M+H]⁺: 662.3024, found: 662.3036.

(1S)-2,3,4,6-Tetra-O-methoxymethyl-1′-N-benzyloxycarbonyl-5′(S)-methyl methoxymethyl ether-spiro[1,5-anhydro-D-glucitol-1,3′-L-proline methyl ester] (2-4) The synthetic procedure is described in SI. [α]_{D}=12.3 (c 1.1, CHCl₃); ¹H NMR (300 MHz, CDCl₃, two isomers): δ=2.23-2.46 (m, 2H), 3.28-3.82 (m, 25H), 3.90 (dd, 0.38H, J=9.1 Hz, J=4.3 Hz), 4.04 (dd, 0.62H, J=8.9 Hz, J=4.4 Hz), 4.16-4.31 (m, 1.62H), 4.36 (s, 0.38H), 4.46-4.84 (m, 10H), 4.91 (d, 0.62H, J=12.6 Hz), 5.14 (brs, 0.76H), 5.22 (d, 0.62H, J=12.6 Hz), 7.23-7.38 (m, 5H); ¹³C NMR (75 MHz, CDCl₃, two isomers): δ=26.8/27.9, 52.0/52.2, 55.2-56.9 (12 carbons), 66.8/67.0, 67.0/67.3, 68.1/68.7, 70.2/70.6, 71.9/72.0, 74.8/74.9, 76.7/76.8, 80.5/80.8, 86.0/87.0, 96.6-98.5 (10 carbons), 127.4-128.4 (10 aromatic carbons), 136.2/136.4 (aromatic carbons), 154.6/155.1, 169.7 (2 carbons); HRMS (ES) calcd for C₃₀H₄₈NO₁₅ [M+H]⁺: 662.3024, found: 662.3034.

(1S)-2,3,4,6-Tetra-O-methoxymethyl-1′-N-benzyloxycarbonyl-5′(R)-methyl methoxymethyl ether-spiro[1,5-anhydro-D-glucitol-1,3′-L-proline] (2-5) To a solution of compound 2-3 (50 mg, 0.076 mmol) in dioxane (3 mL) was added 2M lithium hydroxide aqueous solution (0.38 mL, 0.76 mmol). The reaction mixture was stirred at 60° C. for 48 hours. Afterwards the solution was cooled to room temperature and neutralized with Amberlite IRC-50S H⁺ ion-exchange resin. The mixture was filtered and concentrated to afford the crude product, which was purified by flash chromatography (ethyl acetate/methanol: 20/1) to get pure product 2-5 (45 mg, 93%). [α]_{D}=53.9 (c 1.0, CH₃OH); ¹H NMR (300 MHz, CD₃OD, two isomers): δ=2.15-2.27 (m, 1H, J=13.9 Hz, J=9.7 Hz), 2.50-2.62 (m, 1H, J=13.9 Hz, J=6.6 Hz), 3.25-3.47 (m, 15H, partially overlapping with solvent), 3.48-3.92 (m, 7H), 4.00-4.40 (m, 3H), 4.59-4.84 (m, 10H), 5.07-5.18 (brs, 2H), 7.27-7.43 (m, 5H); ¹³C NMR (75 MHz, CD₃OD, two isomers): δ=32.9/40.0, 55.5-57.2 (12 carbons), 67.9/68.4, 68.3 (2 carbons), 70.6/71.4, 72.8/73.3, 73.3 (2 carbons), 76.7/76.9, 77.9 (2 carbons), 81.0/81.4, 86.3/86.8, 97.6-99.8 (10 carbons), 128.4-129.6 (10 aromatic carbons), 138.2/137.8 (aromatic carbons), 156.8 (2 carbons), 174.0 (2 carbons); HRMS (ES) calcd for C₂₉H₄₄NO₁₅ [M−H]⁻: 646.2711, found: 646.2723.

(1S)-2,3,4,6-Tetra-O-methoxymethyl-1′-N-benzyloxycarbonyl-5′(S)-methyl methoxymethyl ether-spiro[1,5-anhydro-D-glucitol-1,3′-L-proline] (2-6) The synthetic procedure is described in SI. [α]_{D}=30.3 (c 1.2, CH₃OH); ¹H NMR (300 MHz, CD₃OD, two isomers): δ=2.35-2.46 (brs, 2H), 3.28-3.82 (m, 23H, partially overlapping with solvent), 4.13-4.31 (m, 2H), 4.56-4.86 (m, 10H), 5.01-5.23 (m, 2H), 7.28-7.43 (m, 5H); ¹³C NMR (75 MHz, CD₃OD, two isomers): δ=28.1/29.1, 55.5-57.4 (10 carbons), 57.7/58.2, 68.0/68.4, 68.4/68.6, 69.3/69.8, 73.1 (4 carbons), 77.5 (2 carbons), 78.2 (2 carbons), 81.9/82.0, 87.3/88.2, 97.6-100.0 (10 carbons), 128.6-129.6 (10 aromatic carbons), 137.8/138.0 (aromatic carbons), 156.8/157.0, 175.3 (2 carbons); HRMS (ES) calcd for C₂₉H₄₄NO₁₅ [M−H]⁺: 646.2711, found: 646.2718.

Dipeptide H-5′(R)-(MOM)GlcTSPro-Val-NMe₂ (2-7) The amino acid TFA.NH₂-Val-NMe₂ (44 mg, 0.16 mmol) was dissolved in DMF (1 mL) and then added to a solution of acid 2-5 (35 mg, 0.05 mmol) and N,N-diisopropylethylamine (70 μL, 0.32 mmol) in DMF (3 mL). The reaction mixture was stirred for 10 minutes at room temperature followed by the addition of TBTU (41 mg, 0.11 mmol) and stirred for another 8 hours. After that the reaction is quenched with water (8 mL) and extracted with ethyl acetate (4×10 mL). The combined organic layer was dried with sodium sulfate and concentrated. The residue was purified by flash chromatography (ethyl acetate/methanol:30/1) to afford the Cbz-protected intermediate, which was dissolved in ethyl acetate (5 mL) and treated with Pd(OH)₂ (20 mg, 20% wt on charcoal) under hydrogenation condition (H₂, 10 psi). The mixture was stirred for one and half hours at room temperature and filtered, concentrated. The resulted crude product was purified by flash chromatography (ethyl acetate/methanol: 20/1) to afford the dipeptide 2-7 (27 mg, 80%). [α]_{D}=27.4 (c 1.2, CH₃OH); ¹H NMR (500 MHz, CD₃OD): δ=0.97 (d, 3H, J=6.9 Hz), 1.01 (d, 3H, J=6.8 Hz), 1.91 (m, 1H), 2.00-2.13 (m, 2H), 2.94 (s, 3H), 3.20 (s, 3H), 3.30-3.42 (m, 15H, partially overlapping with solvent), 3.46-3.69 (m, 9H), 3.85 (d, 1H, J=12.0 Hz), 4.55 (d, 1H, J=5.3 Hz), 4.62-4.82 (m, 10H); ¹³C NMR (75 MHz, CD₃OD): δ=18.8, 19.5, 31.7, 31.9, 36.1, 38.1, 55.5, 55.6, 55.7, 56.6, 56.7, 57.0, 57.2, 68.5, 70.7, 71.1, 74.3, 77.5, 78.5, 83.7, 89.2, 97.7, 97.9, 99.6, 99.8, 100.1, 172.8, 174.0; HRMS (ES) calcd for C₂₈H₅₄N₃O₁₃ [M+H]⁺: 640.3657, found: 640.3646.

Dipeptide H-5′(S)-(MOM)GlcTSPro-Val-NMe₂ (2-8) The synthetic procedure is described in SI. [α]_{D}=54.0 (c 1.0, CH₃OH); ¹H NMR (500 MHz, CD₃OD): δ=0.95-0.97 (m, 6H), 1.78 (dd, 1H, J=13.2 Hz, J=6.8 Hz), 2.00-2.07 (m, 1H), 2.48 (dd, 1H, J=13.2 Hz, J=8.2 Hz), 2.97 (s, 3H), 3.20 (s, 3H), 3.30-3.42 (m, 14H, partially overlapping with solvent), 3.46-3.72 (m, 9H), 3.80 (s, 1H), 3.89 (d, 1H, J=11.4 Hz), 4.57 (d, 1H, J=4.6 Hz), 4.63-4.84 (m, 10H, partially overlapping with solvent); ¹³C NMR (75 MHz, CD₃OD): δ=18.4, 19.6, 31.9, 36.1, 36.2, 38.0, 55.4, 55.7, 55.8, 56.8, 57.0, 57.3, 58.0, 68.7, 70.2, 73.0, 76.1, 78.4, 79.3, 83.4, 88.0, 97.9 (2 carbons), 99.7, 99.9, 100.2, 171.4, 173.6; HRMS (ES) calcd for C₂₈H₅₄N₃O₁₃ [M+H]⁺: 640.3657, found: 640.3649.

Tripeptide H-D-Phe-5′(R)-(MOM)GlcTSPro-Val-NMe₂ (2-9) To a solution of dipeptide 2-7 (30 mg, 0.05 mmol) and N,N-diisopropylethylamine (43 μL, 0.23 mmol) in DMF (3 mL) was added the D-Fmoc-Phe-OH (56 mg, 0.14 mmol) and PyBOP (73 mg, 0.14 mmol). The mixture was stirred for 18 hours at room temperature before addition of sodium bicarbonate (16 mg, 0.18 mmol). The resulted mixture was dilutated with water (5 mL) and extracted with ethyl acetate (5×10 mL). The combined organic layers were dried (Na₂SO₄) and concentrated. The crude product was purified by flash chromatography (ethyl acetate/methanol:20/1) to provide the Fmoc-protected intermediate, which was dissolved in a mixture of piperidine and DMF (0.2 mL+0.8 mL) and stirred for 1 hour at room temperature. The solution was concentrated and purified by flash chromatography (ethyl acetate/methanol:10/1 to 6/1; TLC was charred with the iodine) to get tripeptide 2-9 (29 mg, 80%). [α]_{D}=2.0 (c 0.4, CH₃OH); ¹H NMR (500 MHz, CD₃OD, two isomers): δ=0.92 (d, 3H, J=6.6 Hz), 1.04 (d, 3H, J=7.0 Hz), 1.98-2.06 (m, 1H), 2.19 (dd, 0.30H, J=14.3 Hz, J=7.8 Hz), 2.32 (dd, 0.70H, J=14.0 Hz, J=11.7 Hz), 2.40-2.47 (m, 2.80H), 2.51-2.60 (m, 1H), 2.67 (dd, 0.30H, J=13.7 Hz, J=7.3 Hz), 2.90-2.94 (m, 1.60H), 3.04 (dd, 0.30H, J=14.0 Hz, J=4.9 Hz), 3.13-3.44 (m, 18H, partially overlapping with solvent), 3.49 (m, 0.30H), 3.55-3.98 (m, 6.70H), 4.22-4.91 (m, 15H), 7.12-7.30 (m, 5H); ¹³C NMR (75 MHz, CD₃OD, two isomers): δ=19.4 (2 carbons), 19.7/19.8, 31.4/35.7, 32.5/32.6, 35.7/36.0, 38.0/38.2, 41.7/42.3, 55.4-56.9 (14 carbons), 57.1/57.8, 66.7 (2 carbons), 68.5/69.5, 72.3/73.3, 72.8/73.5, 75.9/76.2, 76.8/77.3, 78.3/78.9, 85.9/86.8, 97.6-99.4 (10 carbons), 127.7-130.8 (10 aromatic carbons), 138.6/139.1 (aromatic carbon), 170.3/170.6, 173.2/173.3, 178.0/179.0; HRMS (ES) calcd for C₃₇H₆₃N₄O₁₄ [M+H]⁺: 787.4341, found: 787.4345.

Tripeptide H-D-Phe-5′(S)-(MOM)GlcTSPro-Val-NMe₂ (2-10) The synthetic procedure is described in SI. [α]_{D}=−13.2 (c 0.9, CH₃OH); ¹H NMR (500 MHz, CD₃OD, two isomers): δ=0.87-1.03 (m, 6H), 1.90-2.11 (m, 1.14H), 2.27-2.44 (m, 1.86H), 2.67 (dd, 0.14H, J=14.9 Hz, J=7.4 Hz), 2.78 (dd, 0.86H, J=12.9 Hz, J=6.1 Hz), 2.91 (s, 0.42H), 2.93 (s, 2.58H), 2.97-3.11 (m, 2H), 3.28-3.48 (m, 15H, partially overlapping with solvent), 3.49-4.01 (m, 9H), 4.14-4.97 (m, 15H), 7.17-7.27 (m, 5H); ¹³C NMR (75 MHz, CD₃OD, trans isomer): δ=18.7, 19.7, 32.5, 32.9, 36.0, 38.1, 41.2, 55.4-56.7 (7 carbons), 57.8, 68.9, 71.7, 72.7, 72.9, 76.2, 76.5, 79.3, 86.6, 97.6-99.4 (5 carbons), 1273-136.6 (5 aromatic carbons), 139.1 (aromatic carbon), 170.5, 173.4, 176.8; HRMS (ES) calcd for C₃₇H₆₃N₄O₁₄ [M+H]⁺: 787.4341, found: 787.4352.

Tetrapeptide Ac-Leu-D-Phe-5′(R)-(MOM)GlcTSPro-Val-NMe₂ (2-11) To a solution of tripeptide 2-9 (25 mg, 0.03 mmol) and N,N-diisopropylethylamine (29 μL, 0.16 mmol) in DMF (3 mL) was added the Fmoc-Leu-OH (34 mg, 0.10 mmol) and TBTU (31 mg, 0.10 mmol). The mixture was stirred for 18 hours at room temperature before addition of sodium bicarbonate (11 mg, 0.13 mmol). The resulted mixture was dilutated with water (5 mL) and extracted with ethyl acetate (5×10 mL). The combined organic layers were dried (Na₂SO₄) and concentrated. The crude product was purified by flash chromatography (dichloromethane/methanol:20/1) to provide the Fmoc-protected intermediate, which was dissolved in a mixture of piperidine and DMF (0.2 mL+0.8 mL) and stirred for 1 hour at room temperature. The solution was concentrated in vacuum. The resulted mixture was dissolved in methanol (2 mL) followed by the addition of pyridine (13 μL, 0.16 mmol) and acetic anhydride (9 μL, 0.10 mmol). The mixture was stirred for 2 hours at room temperature.

The solution was concentrated and purified by flash chromatography (methylene chloride/methanol: 25/1 to 15/1; TLC was charred with the iodine) to get tetrapeptide 2-11 (25 mg, 92%). [α]_{D}=−19.8 (c 1.15, CH₃OH); ¹H NMR (500 MHz, CD₃OD, two isomers): δ=0.72-1.46 (m, 15H), 1.89-1.92 (m, 3H), 2.03 (m, 1H), 2.26 (dd, 0.29H, J=14.4 Hz, J=8.2 Hz), 2.35 (dd, 0.71H, J=14.2 Hz, J=11.9 Hz), 2.47 (s, 2.13H), 2.51 (dd, 0.71H, J=14.2 Hz, J=6.9 Hz), 2.68 (m, 1H), 2.83 (dd, 0.29H, J=14.5 Hz, J=8.9 Hz), 2.92 (s, 0.87H), 3.04-3.45 (m, 19H, partially overlapping with solvent), 3.46-3.76 (m, 4.71H), 3.84-3.89 (m, 1.71H), 3.95-3.97 (m, 0.29H), 4.05 (d, 0.29H, J=5.2 Hz), 4.09 (dd, 0.29H, J=10.4 Hz, J=5.2 Hz), 4.19-4.45 (m, 2.71H), 4.54-4.84 (m, 12.71H, partially overlapping with solvent), 5.41-5.45 (m, 0.29H), 7.08-7.25 (m, 5H); ¹³C NMR (75 MHz, CD₃OD, two isomers): δ=18.5/19.3, 19.7/19.8, 21.9/22.2, 22.5/22.6, 23.2/23.4, 25.7/25.8, 40.0 (2 carbons), 32.3/32.6, 38.0/38.2, 38.8 (2 carbons), 42.2/42.6, 52.6/53.4, 53.1/53.9, 55.4-56.9 (14 carbons), 57.3/57.6, 66.7 (2 carbons), 68.5/69.6, 72.1/73.4, 72.7/73.6, 75.6/76.5, 76.7/77.6, 80.1 (2 carbons), 85.9/86.5, 97.5-99.7 (10 carbons), 127.7-130.9 (10 aromatic carbons), 137.8/138.4 (aromatic carbon), 170.4/170.6, 172.6/172.9, 173.2/173.3, 173.8/173.9, 174.1/174.9; HRMS (ES) calcd for C₄₅H₇₆N₅O₁₆ [M+H]⁺: 942.5287, found: 942.5293.

Tetrapeptide Ac-Leu-D-Phe-5′(S)-(MOM)GlcTSPro-Val-NMe₂ (2-12) The synthetic procedure is described in SI. [α]_{D}=29.2 (c 0.8, CH₃OH); ¹H NMR (500 MHz, CD₃OD, two isomers): δ=0.81-1.01 (m, 12H), 1.20-1.49 (m, 3H), 1.94-2.06 (m, 4H), 2.11 (dd, 0.23H, J=14.1 Hz, J=8.8 Hz), 2.29-2.46 (m, 1.77H), 2.85-3.19 (m, 8H), 3.30-3.54 (m, 16H, partially overlapping with solvent), 3.66-3.85 (m, 6.31H), 3.94 (d, 0.23H, J=4.5 Hz), 4.0 (d, 0.23H, J=4.6 Hz), 4.08 (dd, 0.23H, J=8.2 Hz, J=4.1 Hz), 4.26-4.88 (m, 14H, partially overlapping with solvent), 4.97 (d, 0.23H, J=6.3 Hz), 5.35 (dd, 0.77H, J=8.5 Hz, J=6.3 Hz), 7.12-7.26 (m, 5H); ¹³C NMR (75 MHz, CD₃OD, two isomers): δ=19.3/19.5, 19.6 (2 carbons), 21.8/22.5, 22.6/22.7, 23.1/23.7, 25.8/25.9, 32.1/32.2, 33.0 (2 carbons), 36.0/36.1, 37.9/38.2, 38.3/38.6, 42.0/42.5, 52.8-57.5 (18 carbons), 69.0/69.3, 71.6/71.7, 72.8 (2 carbons), 73.7 (2 carbons), 75.7/76.5, 76.0/76.7, 77.2/78.9, 86.6/88.3, 97.0-99.1 (10 carbons), 127.6-130.6 (10 carbons), 138.6 (2 carbons), 170.3/171.3, 172.3/172.6, 173.2 (2 carbons), 173.7 (2 carbons), 174.2/174.4; HRMS (ES) calcd for C₄₅H₇₆N₅O₁₆ [M+H]⁺: 942.5287, found: 942.5281.

Tetrapeptide Ac-Leu-D-Phe-5′(R)-GlcTSPro-Val-NMe₂ (2-13) To a solution of compound 11 (30 mg, 0.03 mmol) in methanol (1 mL) was added a 6 M HCl solution (100 μL). The mixture was stirred for 18 hours at room temperature and then concentrated. The crude product was purified by flash chromatography (methylene chloride/methanol: 5/1) to afford unprotected tetrapeptide 2-13 (20 mg, 90%). ¹H NMR (500 MHz, D₂O, cis isomer): δ=0.63-1.32 (m, 15H), 1.90 (s, 3H), 1.94-1.99 (m, 1H), 2.30-2.46 (br, 5H), 2.74-2.79 (dd, 1H, J=12.6 Hz, J=12.9 Hz), 2.94-3.00 (dd, 1H, J=17.5 Hz, J=14.5 Hz), 3.17 (s, 3H), 3.44-3.53 (br, 3H), 3.64-3.87 (m, 5H), 4.04-4.11 (m, 1H), 4.17-4.25 (m, 1H), 4.34-4.40 (m, 1H), 4.59 (m, 1H), 4.77 (1H, overlapping with solvent), 7.10-7.38 (m, 5H); ¹³C NMR (75 MHz, CD₃OD, cis isomer): δ=19.4, 19.7, 22.1, 22.3, 23.2, 25.7, 25.9, 31.8, 35.9, 38.2, 38.5, 42.0, 53.0, 54.2, 56.6, 59.9, 60.8, 62.6, 71.0, 71.2, 73.2, 75.3, 77.3, 87.3, 127.7-130.4 (5 carbons), 138.5, 171.7, 173.1, 174.1, 174.5, 174.8; HRMS (ES) calcd for C₃₅H₅₆N₅O₁₁ [M+H]⁺: 722.3976, found: 722.3985.

ROESY experiments. ROESY spectra of tetrapeptides 2-11 to 2-13 and 2-16 were recorded in AMX 500 with 256 points in t1, 2048 points in t2 and 64 scans per t2 increment.

Temperature coefficient experiments. 1 D ¹H-NMR spectroscopy of 8 mM solutions of 2-11, 2-12 and 2-16 in 100.0% Me₂SO-d₆ and 2-13 in H₂O/D₂O (9/1) were recorded on a Bruker AMX500 at 20° C., and from 20 to 45° C. with increments of 5° C., using routine techniques. Chemical shift (δ) are expressed in ppm and calibrated with respect to the residual DMSO signal (1H, 2.49 ppm) or the TSP (1H, 0.00 ppm) in water.

### Example 3

### Design and Synthesis of Glucose-Templated Proline-Lysine Chimera: Polyfunctional Amino Acid Chimera with High Prolyl cis Amide Rotamer Population

### Abstract

The inventors describe the synthesis of two glucose-templated proline-lysine chimeras (GlaProLysCs) that differ in the stereochemistry of the hydroxymethyl substituent at the C-5′ position of the pyrrolidine ring. The key synthetic steps involve C-glycosylation of an exocyclic glucose-based epoxide with allyltributylstannane that affords functionalized C-ketosides containing an α-hydroxy ester moiety, introduction of an amino group at C-2 through stereoselective reductive amination, and regioselective installation of the azide group at C-6 on the glucose scaffold. Incorporation of these chimeras into the model peptides Ac-GlcTProLysC-NHMe and Ac-GlcTProLysC-OMe demonstrates that the stereochemistry of the hydroxymethyl substituent at the C-5′ position has a profound effect on the equilibrium constant of prolyl amide cis/trans isomerization. The equilibrium constant Kₐₜ for the peptide mimic Ac-GlcTProLysC-NHMe with C-5′(R) stereochemistry was determined to be 3.03±0.04 while the K_{t/c} for the C-5′(S) diastereoisomer was 0.56±0.04 in D₂O. Temperature coefficient experiments indicate the origin of these effects are derived from two critical hydrogen bonds involving the C-5′ hydroxymethyl substituent; one to the N-terminal amide carbonyl group, and a second to the primary amino group in the glucose moiety.

### Introduction

Conformationally constrained amino acids have found wide applications as building blocks to study and probe the bioactive conformation of peptides when binding to receptors (Gellman, 1998; Belec et al., 2000; An et al., 1999). Among all naturally occurring amino acids, proline is the only amino acid with a side chain fused onto the peptide backbone. Its cyclic structure restricts the rotation about its φ dihedral angle, thereby reducing the energy difference between the prolyl amide cis and trans isomers. Thus, while most peptide amide bonds exist almost exclusively in the trans form, proline has a much greater propensity to form cis amide bonds. A variety of factors influence cis/trans isomerization of proline; these include electron withdrawing groups attached to the pyrrolidine ring (Eberhardt et al., 1996; Improta et al., 2001; Song et al., 2005), n→π* interaction (Hinderaker and Raines, 2003), Ar-Pro interactions (Wu and Raleigh, 1998; Thomas et al., 2006; Halab and Lubell, 2002) and steric effects (Beausoleil and Lubell, 1996). In particular, incorporation of bulky substituents into the 5-position of proline has been shown to enhance the prolyl amide cis population significantly. Cis/trans isomerization of proline plays an important role in the formation of secondary structures in peptides and proteins because proline induces a reversal in backbone conformation resulting in the formation of reverse turns and disruption of helices and sheets in proteins. Besides the occurrence of proline in β-turns, proline-rich sequences also exist as extended helices (Kakinoki et al., 2005) (polyproline-I and polyproline-II) and antimicrobial peptides (Reddy et al., 2004). Furthermore, proline undergoes post-translational modifications to form 4-(R)-hydroxyprolines, which are known to contribute to the enhanced stability of the polyproline-II conformation in both collagenous proteins and peptides (Eberhardt et al., 1996; Improta et al., 2001; Song et al., 2005), and plant cell wall glycoproteins (Cooper et al., 1987).

Proline analogues displaying the characteristics of other amino acids are referred to as proline-amino acid chimera, and have been used to study the spatial requirements for receptor affinity and biological activity of both natural amino acids (Baldwin et al., 1995; Hashimoto et al., 1994; Bridges et al., 1991; Tsai et al., 1988) and peptides (Kolodziej et al., 1995; Holladay et al., 1991; Plucinska et al., 1993; Ghose et al., 1995; Mosberg et al., 1992; Mosberg et al., 1994). For example, β-substituted-prolines such as 3-carboxyproline,^{11a} 3-phenylproline (Chung et al., 1990) and 3-di-methylproline (Sharma and Lubell, 1996) combine amino acid side-chain functionality with proline conformational rigidity. In these cases, replacement of the natural amino acids in peptides by proline-amino acid chimeras provided better understanding of the bioactive conformations of peptides binding to receptors (Kolodziej et al., 1995; Holladay et al., 1991; Plucinska et al., 1993; Ghose et al., 1995; Mosberg et al., 1992; Mosberg et al., 1994). While these analogues have proved useful for inducing specific constraints into amino acids and peptides, their structures do not permit additional derivatization; a trait that is often required in drug discovery and lead optimization. Polyfunctional proline-amino acid chimera may overcome these drawbacks.

The concept for developing such polyfunctional proline-amino acid chimera was derived from glycosyl amino acids (GAAs) which are defined by an α-amino acid group [CH(NH₂)CO₂H] either directly attached or carbon-linked to the anomeric carbon of a carbohydrate scaffold (Dondoni and Marra, 2000; Schweizer, 2002; Gruner et al., 2002). The relative rigidity of the pyran ring combined with the polyfunctional nature of the carbohydrate scaffold has inspired the design of unusual and conformationally constrained amino acids and novel peptidomimetics (Dondoni and Marra, 2000; Schweizer, 2002; Gruner et al., 2002).⁸ While there are many examples of C-glycosylglycine, -alanine, -serine, and -asparagine (Dondoni and Marra, 2000), few proline-based GAAs exist (Owens et al., 2007; Zhang and Schweizer, 2005; Cipolla et al., 2005).

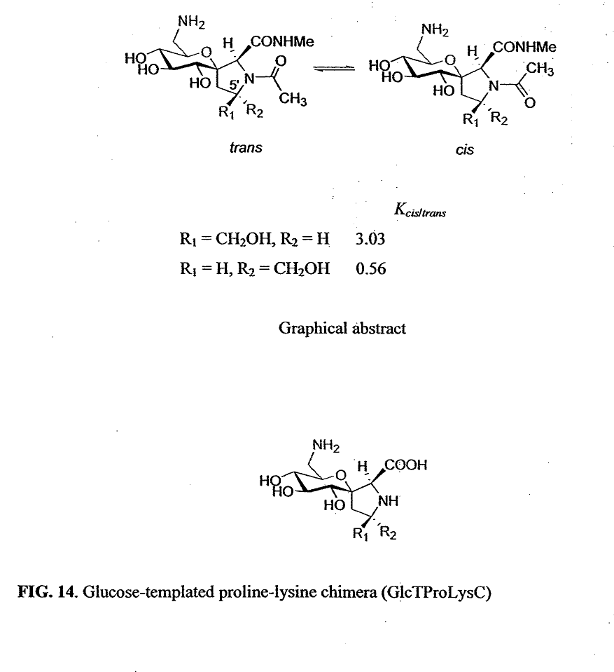

The inventors report here on the design, synthesis of spirocyclic glucose-templated proline-lysine chimeras (GlcTProLysCs) and describe their properties in peptide mimics. Spirocyclic GlcTProLysC were selected on the basis of previous synthetic methodology (Zhang et al., 2008). Bicyclic GlcTProLysCs combine the molecular features of glucose (pyran-based polyol) with the unique characteristics of proline or 3-hydroxyproline and L-lysine (FIG. 14). The characteristics of the lysine side chain including relative length and presence of amino function are presented on the pyrrolidine ring and are further constrained by incorporation into the 6-amino-6-deoxy-D-glucose scaffold. Proline-lysine chimera were selected due to its frequent occurrence in cationic antimicrobial peptides with polyproline conformation (Reddy et al., 2004). In order to control the prolyl amide cis/trans isomerization the inventors were interested in developing GlcTProLysC analogs that contain hydrogen bond forming substituents like a hydroxymethyl group at the δ-position of proline.

Previous studies have shown that bulky substituents at the δ-position (including δ-tert-butyl proline (Beausoleil and Lubell, 1996) and δ,δ-dimethyl proline (An et al., 1999) enhance the prolyl amide cis isomer population. However, hydrogen bond forming groups at the δ-position have never been investigated. In addition, chemical manipulations and derivatization of the polyol scaffold provides an opportunity to adjust the chemical, physical and pharmacodynamic properties of proline-containing peptides (Haubner et al., 2001). This may provide a novel tool to functionalize extended helical structures including PP1 and PP2 (Kuemin et al., 2007; Shi et al., 2006). Moreover, incorporation of polyhydroxylated amino acids have been shown to induce novel secondary structures in small peptides. For instance, incorporation of unprotected sugar amino acids into small peptides such as gramicidin S (Grotenberg et al., 2004) and opioid peptides (Chakraborty et al., 1998) have prohibited the formation of the targeted secondary structural motif. Instead, unusual turn structures were stabilized by intramolecular hydrogen bonds between sugar hydroxyl groups and the peptidic amide backbone. Similar effects may also be observed with GlcTProLysC.

### 2. Results and Discussion

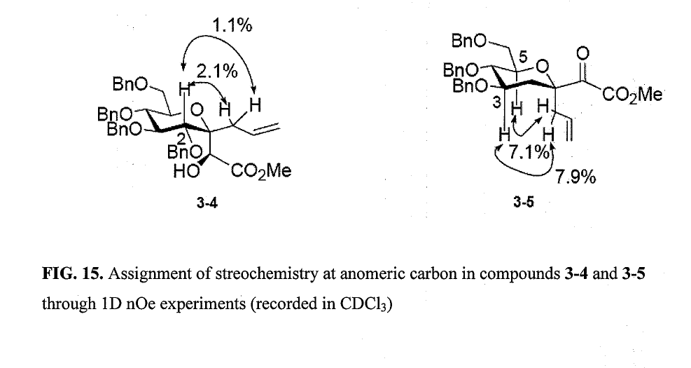

The synthesis started with the readily available D-gluco-based lactone 3-1 (Gueyrard et al., 2005) (Scheme 5) which reacts with the enolate of methyl bromoacetate generated from lithium bis-(trimethylsilyl)amide (LiN(SiMe₃)₂) in tetrahydrofuran (THF) at −78° C., to produce the exocyclic epoxide 3-2 in 80% yield as a single stereoisomer (Schweizer and Inazu, 2001; Zhang et al., 2007). Trimethylsilyl trifluoromethanesulfonate (TMSOTf)-promoted C-glycosylation of epoxide 3-2 with allyltributylstannane in dichloromethane followed by hydrolysis of the TMS-ether with trifluoroacetic acid (TFA)-containing wet THF produced a mixture containing alcohols 3-3 and 3-4 (ratio 3-3: 3-4=9:1) in a combined yield of 89%. Regioselective opening of epoxide 3-2 proceeded via formation of oxonium ion (intermediate A) that subsequently undergoes α-selective C-glycosylation favored by stereoelectronic factors as observed for similar C-glycosylation reactions (Dillon et al., 1995). It is noteworthy that slow addition of epoxide 3-2 to allyltributylstannane is crucial for optimal yield of target compound 3-3. The configuration at the anomeric position in compound 3-4 was deduced on the basis of observed/unobserved nOe contacts (FIG. 15).

Compound 3-3 served as starting material for the installation of the amino function at C-2 (Scheme 6). Initially, the inventors attempted to convert the hydroxyl group at C-2 into an amino function. However, nucleophilic substitution of C-2 activated sulphonate ester (triflate) with a variety of nucleophiles including benzylamine, p-methoxybenzylamine, lithium and sodium azide at low and elevated temperatures resulted only in trace amounts of the desired amine. In these cases, unreacted starting material was recovered (>90%). To avoid these complications the inventors decided to explore a reductive amination approach. Alcohol 3-3 was oxidized to ketone 3-5 at −78° C. using a mixture containing trifluoroacetic anhydride, triethylamine and dimethylsulfoxide in dichloromethane to produce ketone 3-5 in 95% isolated yield (Huang et al., 1976).

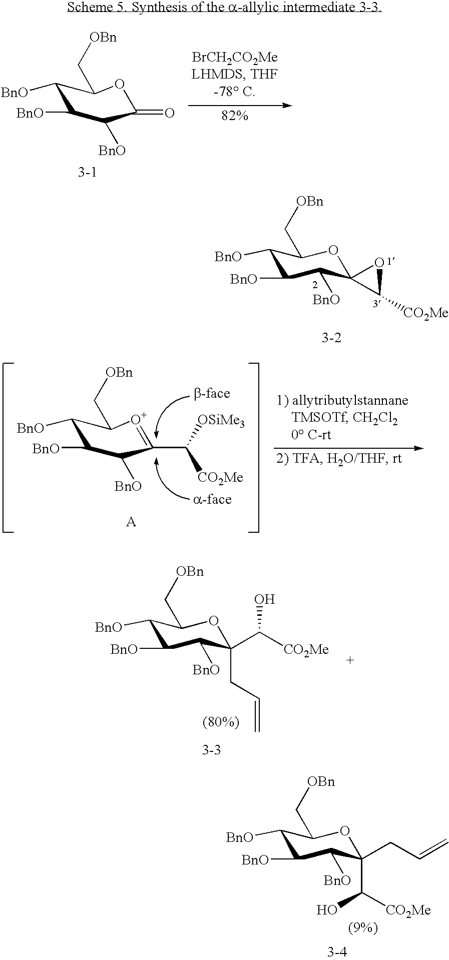

In order to confirm the configuration of the product, the inventors performed nOe experiments (FIG. 15). For instance, subjection of one of the allylic protons to a one-dimensional GOESY experiment showed interproton effects to H-3 (7.9% nOe) and H-5 (7.1%). This is consistent with the structure 3-5 bearing an allylic group at the axial position.

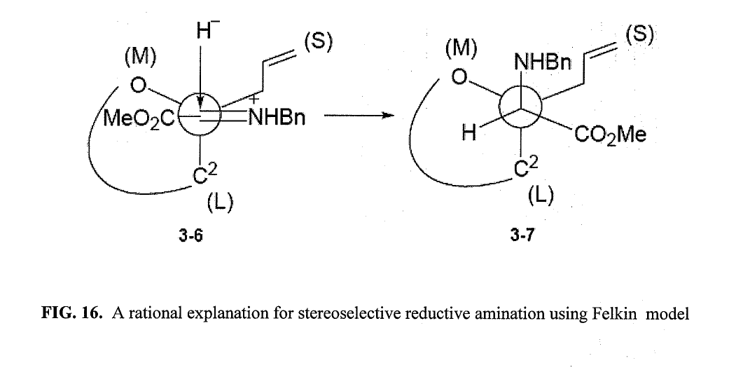

Subsequently, the ketone 3-5 was converted into the amino ester 3-7 in a two-step procedure. At first, compound 3-5 was exposed to titanium tetrachloride-promoted imination using benzylamine in ether to afford the imine 3-6 in 96% after chromatographic purification (Boeykens et al., 1994). The imine 3-6 was stereoselectively reduced to amino ester 3-7 in quantitative yield using sodium cyanoborohydride in acidified methanol at 0° C. The high diastereoselectivity could be explained in the Felkin model (FIG. 16) (Cherest et al., 1968), in which the nucleophile approached to the imine ion from the less hindered side (Re face) and resulted in the formation of S-configuration at C-2 position of compound 3-7, which was confirmed by the following nOe experiments.

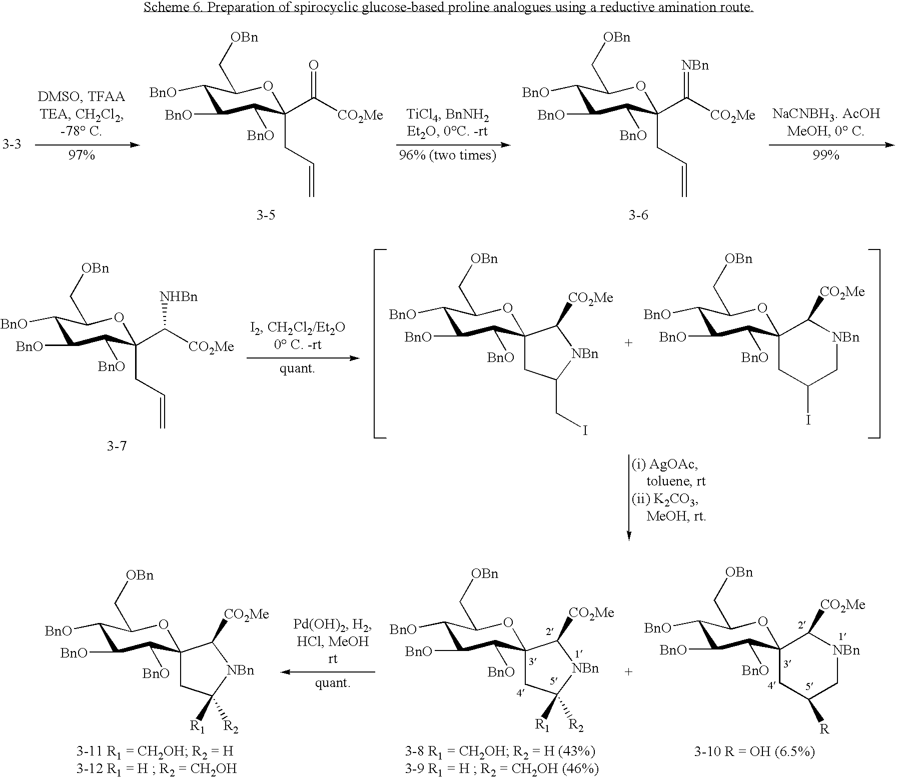

With amino ester 3-7 in hand the inventors installed the pyrrolidine ring by iodocyclization in dichloromethane to produce an inseparable isomeric mixture containing various iodo-compounds. To separate the compounds from each other the inventors converted the mixture into the alcohols 3-8, 3-9 and 3-10 via a two-step process. At first, the mixture was exposed to silver acetate in toluene³³ to produce an inseparable mixture of esters 3-13, 3-14 and 3-15 (Scheme 6) that, by treatment with potassium carbonate in methanol, afforded the alcohols 3-8, 3-9 and 3-10 in 44%, 45% and 6% isolated yield, respectively. Subsequently, exposure of compounds 3-8 and 3-10 to catalytic hydrogenolysis condition using Pearlman's catalyst provided the unprotected proline analogues 3-11 and 3-12 in quantitative yield, respectively.

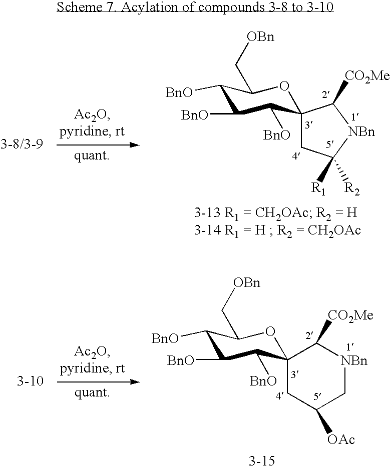

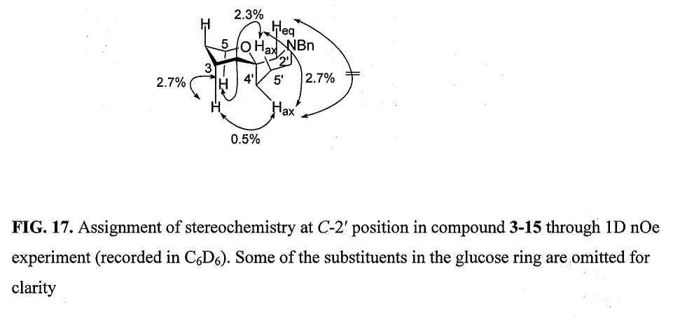

To assign the stereochemistry at C-2′ the alcohols 3-8, 3-9 and 3-10 were converted into the acetates 3-13, 3-14 and 3-15 using standard conditions (acetic anhydride in pyridine, Scheme 7). The inventors selected the pipecolic, acid analogue 3-15 to assign the stereochemistry at C-2′ (FIG. 17). The spirocyclic compound 3-15 consists of both a pyranose and a piperidine ring. The large coupling constants for J_{2,3}, J_{3,4} and J_{4,5} (>9.0 Hz) in conjunction with interproton nOe effects between H-3 and H-5, establishes the ⁴C₁ chair conformation of the sugar ring. The chair conformation of the piperidine ring is deduced from the observed vicinal diaxial and long-range coupling constants. For instance, the axial position of protons H-4′ₐₓ, H-5′ₐₓ and H-6′ₐₓ can be deduced by their large vicinal diaxial coupling constants (J_{4′ax,5′ax}, J_{5′ax,6′ax}>10.5 Hz), while the observed long-range coupling constants between J_{4′eq,6′eq} is equal to J_{2′eq,4′eq} (˜1.0 Hz); confirming the equatorial position of H-2′_{eq}, H-4′_{eq} and H-6′_{eq} in the piperidine ring. In addition, the observed interproton effects (nOe) between H-5/H-5′ₐₓ, H-5/H-3, and H-3/H-4′ₐₓ, together with the unobserved effect between H-6′ₐₓ/H-2′_{eq} using a one-dimensional GOESY experiment, determines the C-2′ (S) configuration (FIG. 17) (Huang et al., 1976; Stonehouse et al., 1994).

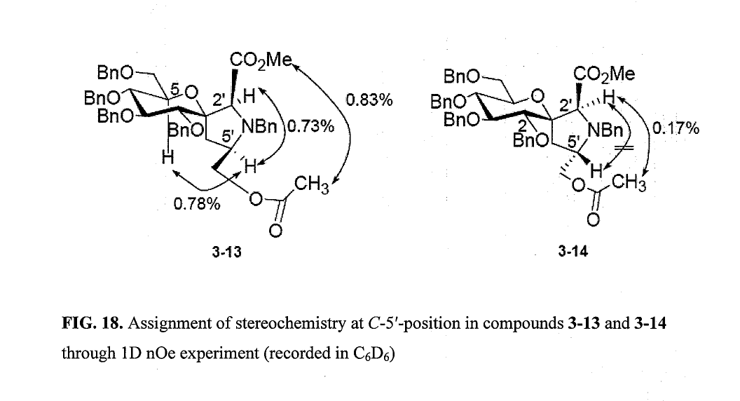

Once the inventors had established the configuration at C-2′ in compound 3-15, the inventors turned the interest to the stereochemistry at C-5′ of the spirocyclic proline analogues 3-13 and 3-14. Since the iodocyclization was performed on a single stereoisomer 3-7, the inventors assume that the stereochemistry at C-2′ of the proline analogues 3-13 and 3-14 remained “S” based on the previous assignment with piperidine analogue 3-15. To discriminate between compounds 3-13 and 3-14 the inventors again used nOe (Huang et al., 1976) experiments (FIG. 18). As an example, the observed interproton effects between H-2′/H-5′ and H-5′/H-5 for compound 3-13 are consistent with the C-5′ (R) stereochemistry. By comparison, proline analogue 3-14 did not show any interproton effect between H-2′/H-5′, which is consistent with a C-5′ (S) configuration.

Once the inventors had established the stereochemistry at the C-2′- and C-5′-positions in compounds 3-13 and 3-14, the inventors then focused on the conversion of the primary hydroxyl group on the glucose moiety into an amino function (Scheme 8). Initially, compounds 3-13 and 3-14 were exposed to catalytic hydrogenolysis condition using Pearlman's catalyst. The resulting N-debenzylated amine was protected using di-tent-butyl dicarbonate and triethylamine in methanol to afford the carbamates 3-16 and 3-17 in 90% and 62% yield respectively without acyl migration. The azido group in compounds 3-16 and 3-17 was installed by a standard two-step procedure: first, selective activation of the primary hydroxyl group as sulfonate ester, followed by nucleophilic substitution with sodium azide in DMF at 80° C., produced azides 3-18 and 3-19 in excellent yields.

Azides 3-18 and 3-19 served as starting materials for incorporation into the peptide mimics 3-22 to 3-25 (Scheme 9), which were used to study the thermodynamic properties of cis/trans isomerization of the glucose-templated proline-lysine chimera. In addition, the inventors selected peptide esters 3-22 and 3-23 bearing a C-terminal methyl ester as well as methyl amides 3-24 and 3-25 as peptide mimics to study how the nature of the C-terminal group affects N-terminal prolyl amide isomerization.

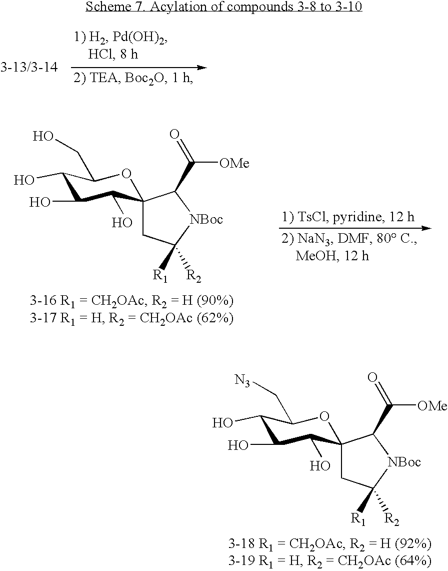

Peptide esters 3-22 and 3-23 are prepared from azides 3-18 and 3-19 using the synthetic route outlined in Scheme 9. Deprotection of the N-Boc group in 3-18 and 3-19 with trifluoroacetic acid followed by acetylation using pyridine and acetic anhydride and O-deacetylation using sodium methoxide in methanol produced azides 3-20 and 3-21 in 96% and 80% yield, respectively. Attempts to perform selective N-acylation failed and produced complex reaction mixtures. Catalytic hydrogenation of azide 3-20 and 3-21 using Pearlman's catalyst produced peptide esters 3-22 and 3-23 in quantitative yield. The N′-methylamides 3-24 and 3-25 were prepared from azides 3-20 and 3-21 through a two-step procedure: first, methyl esters 3-20 or 3-21 were treated with a concentrated methylamine solution in ethanol to afford N′-methylamide intermediates. Subsequently, the azido function was reduced by catalytic hydrogenation to produce peptide mimics 3-24 and 3-25 in 93% and 95% yield, respectively.

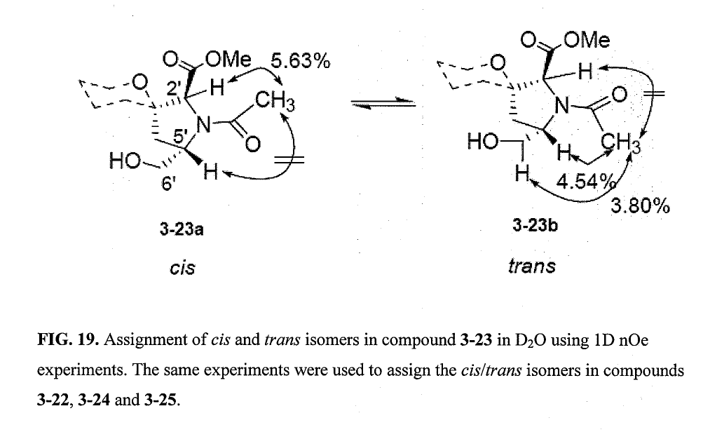

The assignments of N-terminal geometry for model peptides 3-22 to 3-25 were made on the basis of nOe experiments in D₂O (FIG. 19). For example, selective inversion of the N-terminal methyl group in the prolyl amide cis isomer 3-23a by a selective GOESY³⁴ experiment showed an interproton effect to H-2′ (5.63% nOe). By comparison, no interproton effect was observed between H-2′ and methyl group of N-terminus in trans isomer 3-23b. Moreover, selective inversion of the methyl group of N-terminus in 3-23b showed interproton effects to H-5′ (4.54% nOe) and H-6′ (3.80% nOe). Similar experiments were performed to assign the cis/trans isomers in compounds 3-22, 3-24 and 3-25.

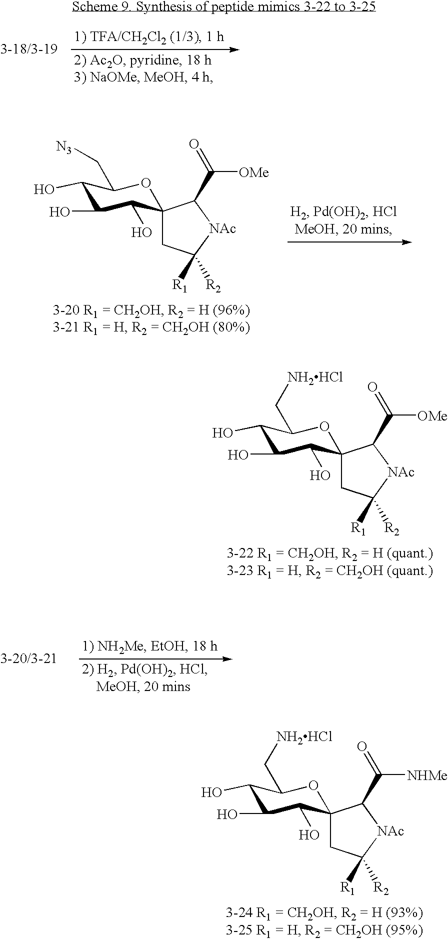

In addition, the inventors observed that the ¹³C NMR chemical shifts of the C^{a} atom of the trans rotamer in compounds 3-22 to 3-25 are high field shifted (0.75-1.02 ppm) relative to the cis isomer in water. This is consistent with previous observations made by Lubell and co-workers⁷ on other proline-containing peptide mimics and may serve as a diagnostic tool to assign the trans isomer in cases where nOe-experiments do not allow assignment due to spectral overlap.

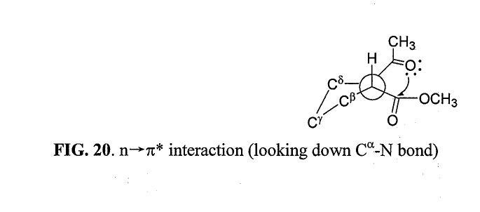

The equilibrium constants K_{c/t} for compounds 3-22 to 3-25 are shown in Table 15 and were determined by integrating and averaging as many distinct proton signals as possible for both the major and minor isomers in the ¹H NMR spectra (Taylor et al., 2003). The results indicate that compounds 3-22 and 3-24 display a higher cis isomer population relative to their C-5′ distereoisomers 3-23 and 3-25, respectively. It appears that the stereochemistry at C-5′ has a profound effect on the equilibrium of isomerization. Also, the cis prolyl amide population in esters 3-22 and 3-23, was generally lower than N′-methylamides 3-24 and 3-25. Taylor and co-workers have proposed that the trans conformation of esters is stabilized relative to amides as a result of increased electron donation from the oxygen lone pair of the N-terminal amide carbonyl group to the antibonding orbital of the prolyl C-terminal carbonyl group (FIG. 20) (Taylor et al., 2003).

TABLE 15
Cis population (%) and equilibrium constant
K_{c/t} of compounds 3-22 to 3-25 in D₂O

| Compds | 3-22 | 3-23 | 3-24 | 3-25 |
|---|---|---|---|---|
| cis(±3%) | 55 | 19 | 75 | 36 |
| K_{c/t}(±0.04) | 1.22 | 0.23 | 3.03 | 0.56 |

Effect of pH. Since compound 3-22 to 3-25 have an ionizable amino group, the inventors were interested to study the influence of the ionization state on K_{t/c}. Previous studies have indicated that ionizable groups in proximity to the imide backbone can influence thermodynamics of prolyl amide cis/trans isomerization (Dugave and Demange, 2003). The inventors selected three buffer ranges: pH=2.6, 7.4 and 12.4, to examine the pH effect on the isomerization of 3-24 and 3-25 (Table 16). The study shows that the prolyl N-terminal amide cis/trans ratio is not affected by pH and the observed changes are within the experimental error. Molecular modeling suggests that the large distance of the ionizable amino function from the imide function is responsible for the absence of a pH effect.

TABLE 16
pH effect on K_{c}_{/t} for compounds 3-24 and 3-25 in D₂O

| | pD | | |
|---|---|---|---|
| Compds | 2.6 | 7.4 | 12.4 |
| 3-24 (±0.04) | 3.03 | 3.03 | 2.70 |
| 3-25 (±0.04) | 0.61 | 0.56 | 0.61 |

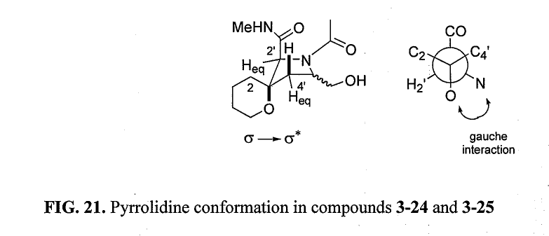

Conformational analysis of compounds 3-24 and 3-25. The relatively large coupling constants for J_{2,3}, J_{3,4} and J_{4,5} (≧9.2 Hz) indicate a ⁴C₁ conformation of the pyranose ring in 3-24 and 3-25. The conformation of the piperidine ring is expected to be C^{β}-exo based on previous studies using 3(S)-hydroxyproline-containing peptide mimics (Jenkins et al., 2003; Taylor et al., 2005). This conformation places the endocyclic oxygen substituent in an axial position (Taylor et al., 2005). In this conformation the pyrrolidine ring will be stabilized by gauche interaction and a stabilizing σ(C^{γ}—H)→σ*(C^{β}—O) interaction. This conformation is further supported by characteristic long range “W” coupling constants (J˜1.0 Hz) between H-2′_{eq} and H-4′_{eq} in both compounds of 3-24 and 3-25 (FIG. 21).

In order to explain the different cis/trans ratio in compounds 3-24 and 3-25 the inventors considered intramolecular hydrogen bonding, which can be studied by calculating the temperature coefficients (Δδ/ΔT) of key exchangeable protons (Leeflang et al., 1992). Previous studies have shown that (Δδ/ΔT)>−3.0 ppb/deg are a diagnostic tool for the detection of intramolecular H-bonding (Leeflang et al., 1992).

The 1D spectra of compounds 3-24 and 3-25 were analyzed between 25 to 45° C. in 5-deg steps in DMSO-d₆ to determine the temperature coefficients (Table 17). The results indicate that the protons associated with NH₂-6, OH-6′ and NHMe exhibit the highest temperature coefficient values suggesting that these protons are involved in intramolecular H-bonding in compounds 3-24 and 3-25. The low (Δδ/ΔT) values observed for OH-2 and OH-3 reflect high solvent exposure of these hydroxyl groups while the relative high value for OH-4 in 3-25 may indicate some H-bond interaction with one of the nitrogen lone pairs on NH₂-6. The relative high and nearly identical (Δδ/ΔT) values observed for the methyl amide proton in structures 3-24a, 3-24b, 3-25a and 3-25b supports the notion that this proton is engaged in hydrogen bonding to N-terminal carbonyl group in cis/trans isomers of both compounds 3-24 and 3-25. Moreover, the higher (Δδ/ΔT) value for the NH₂-6 in compound 3-25 when compared to diastereomer 3-24 suggests that the amino group in 3-25 is involved in stabilization of the trans isomer 3-25b and the destabilization of the cis isomer 3-25a relative to 3-24a and 3-24b.

TABLE 17
Temperature coefficients (Δδ/ΔT, ppb/K)
for compounds 3-24 and 3-25 in DMSO-d₆

| | | HO-2 | HO-3 | HO-4 | NH₂-6 | HO-6′ | NHMe |
|---|---|---|---|---|---|---|---|
| 3-24 | cis | −9.07 | ^{[a]} | ^{[a]} | −2.44^{[b]} | −3.80 | −2.44^{[b]} |
| | trans | −8.93 | ^{[a]} | ^{[a]} | −2.44^{[b]} | −4.28 | −3.02 |
| 3-25 | cis | −8.09 | −6.90 | −4.10 | −0.92 | −3.29 | −2.68 |
| | trans | −7.76 | −6.80 | −4.20 | −0.92 | −2.76 | −3.26 |

^{[a]}not determined;
^{[b]}overlapped with NHMe.

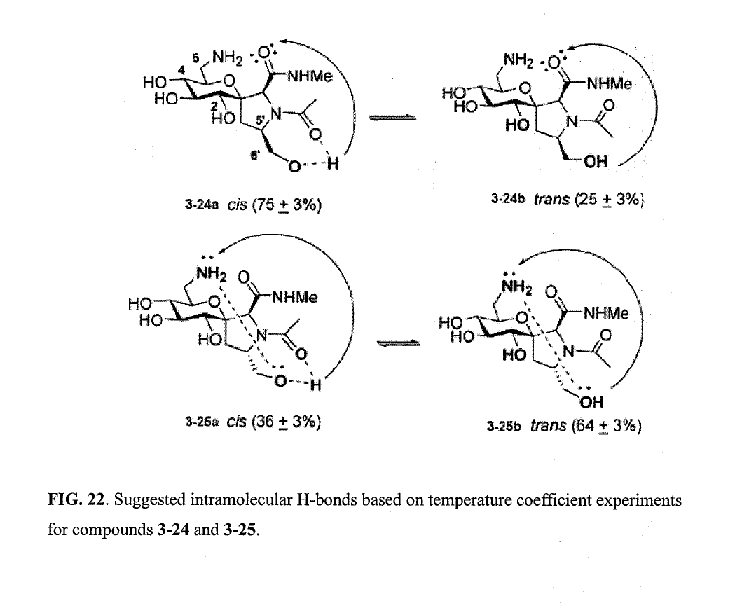

A similar trend is observed for the OH-6′ position. Isomers 3-25a and 3-25b exhibit higher (Δδ/ΔT) values when compared to isomers 3-24a and 3-24b suggesting that OH-6′ is involved in stabilization of the trans isomer 3-25b relative to 3-24b. Taken together these results support the notion that compound 3-25 is stabilized by an intramolecular hydrogen bond (6′-OH—NH₂-6) that is absent in 3-24. The hydrogen bond (OH-6′-NH₂-6) competes with the H-bond (6′-OH O═C(N)CH₃) of the cis isomer 3-25a resulting in a lower cis population of 3-25a relative to 3-24a (FIG. 22).

### Conclusions

The inventors have developed a synthetic route to two spirocyclic GlcTProLysCs that differ in the stereochemistry of the hydroxymethyl substituent at the C-5′ position of the pyrrolidine ring. A key intermediate in the synthesis is the glucose-templated C-glycosyl glycine analog 3-7 that bears an additional C-allylic substituent at the pseudoanomeric position. Compound 3-7 may find future use in the synthesis of other carbohydrate-templated amino acids via derivatization of the allyl group. In order to study the thermodynamic properties of prolyl amide cis/trans isomerization the two GlcTProLysC analogs were incorporated in peptide mimics Ac-GlcTProLysC-OMe and Ac-GlcTProLysC-NHMe.

The study indicates that the stereochemistry at the C-5′ position in both peptide mimics has a profound effect on the equilibrium constant. For example, incorporation of GlcTProLysC with “R” configuration at C-5′ dramatically increased the cis population (75%) of Ac-GlcTProLysC-NHMe in water, whereas a smaller augment cis population (34%) was observed in GlcTProLysC with “S” configuration at C-5′. Temperature coefficient experiments indicate that the hydroxymethyl group at C-5′ (S) is involved in H-bonding with the 6-NH₂ and vice versa. In contrast, the same hydrogen bond is absent in the diastereomer with C-5′ (R) stereochemistry. Taken together the results suggest that the cis isomer ratio in peptide mimic Ac-GlcTProLysC-NHMe having a C-5′ (S) hydroxymethyl substituent is decreased by competing H-bonding effects between 6′-OH—-O═C(N)CH₃ and 6′-OH—-6-NH₂.

The work shows for the first time that polar groups capable of H-bonding in polyhydroxylated spirocyclic proline analogs can play important roles to control the thermodynamics of prolyl amide cis/trans isomerization. In particular, polar groups such as a hydroxymethyl group introduced at the δ-position of proline are expected to increase the prolyl N-terminal amide cis isomer in peptides via H-bonding to the N-terminal amide carbonyl group. Previous studies have shown that amino acid residues possessing side chains with hydrogen-bond acceptor and donor moieties are able to stabilize turn conformations when adjacent to proline (Wilmot and Thornton, 1988; Marraud and Aubry, 1984). As a result the inventors expect that incorporation of GlcTProLysC in bioactive peptides may induce similar effects. The inventors are currently studying the lysine- and proline-mimetic effects of GlcTProLysC in β-turn-forming peptides.

### Experimental Section

General. All solvents were obtained from a dry solvent system (alumina) and used without further drying. TLC was performed on E. Merck Silica Gel 60 F254 with detection by charring with 8% H₂SO₄ acid. Silica gel (0.040-0.063 mm) was used for column chromatography. Melting points are uncorrected.

(1R)-2,3,4,6-Tetra-O-benzyl-3′(S)-carboxy methyl-spiro[1,5-anhydro-D-glucitol-1,2′-oxirane] (3-2). Under nitrogen atmosphere, methyl bromoacetate (4.1 mmol) was dissolved in dry THF (20 mL) and cooled to −78° C. before lithium bis(trimethylsilyl)-amide (4 mL of a 1 M solution in THF) was slowly added. The reaction mixture was kept at −78° C. for an additional 30 minutes. Subsequently, a THF solution (5 mL) containing the lactone 3-1 (1 mmol) was added over a period of 10 minutes and kept at −78° C. for 1 more hour. The temperature was raised to room temperature and stirred for 15 minutes before a saturated aq. NH₄Cl solution was added. The reaction mixture was evaporated under reduced pressure and the residue was dissolved in dichloromethane and portioned with water (3×20 mL). The organic layer was dried over anhydrous Na₂SO₄, concentrated and purified by flash column chromatography (hexanes/ethyl acetate: 5/1) to get 3-2 as a colorless oil (500 mg, 82%). [α]_{D}=99.6 (c 1.0, CHCl₃); ¹H NMR (500 MHz, CDCl₃): δ=3.53 (m, H-7), 3.63 (dd, H-8a, J=11.2 Hz, J=2.1 Hz), 3.68 (dd, H-8a, J=11.2 Hz, J=4.4 Hz), 3.72 (s, 3H), 3.75-3.83 (m, H-5, H-6), 3.90 (s, H-2), 3.95 (d, H-4, J=9.1 Hz), 4.48 (d, 1H, J=12.2 Hz), 4.55 (d, 1H, J=10.7 Hz), 4.58 (d, 1H, J=12.2 Hz), 4.62 (d, 1H, J=11.0 Hz), 4.73 (d, 1H, J=11.0 Hz), 4.82 (d, 1H, J=11.2 Hz), 4.84 (d, 1H, J=10.7 Hz), 4.94 (d, 1H, J=11.2 Hz), 7.13-7.36 (m, 20H); ¹³C NMR (75 MHz, CDCl₃): δ=52.5, 54.5, 68.3, 73.5, 74.9, 75.2, 75.7, 76.8, 77.0, 77.3, 84.7, 86.29, 127.6-128.5 (aromatic carbons), 137.4, 137.8, 137.9, 138.3, 166.7; Anal. calcd for C₃₇H₃₈O₈: C, 72.77; H, 6.27. Found: C, 72.48; H, 6.54. MS (ES, [M+Na]⁺) calcd for C₃₇H₃₈NaO₈ 633.25, found 633.71

Methyl (2S)-hydroxy-2-(1-allyl-2,3,4,6-tetra-O-benzyl-β-D-glucopyranosyl)-ethanoate (3-3). Under a nitrogen atmosphere, to a solution of allyltributylstannane (0.99 mL, 3.15 mmol) in dichloromethane (5 mL) was added dropwise the solution of trimethylsilyltrifluoromethanesulfonate (TMSOTf, 0.427 mL, 2.36 mmol) in dichloromethane (5 mL) at 0° C. followed by the syringe pump-controlled (50 μL/min) addition of the solution of epoxide 3-2 (480 mg, 0.79 mmol) in dichloromethane (10 mL). And then the mixture was stirred for 1 more hour at room temperature, the saturated sodium bicarbonate solution (10 mL) was added to quench the reaction, followed by the extraction with dichloromethane (3×15 mL). The organic layer was dried (Na₂SO₄), concentrated and treated with trifluoroacetic acid (0.20 mL, 5 equiv.) in aqueous tetrahydrofuran (THF/H₂O:5/1) overnight. The mixture was concentrated and purified by flash column chromatography (hexanes/dichloromethane/ethyl acetate: from 2/1/0.2 to 2/1/0.4) to get 3-3 (410 mg, 80%) and 3-4 (46 mg, 9%). (3-3) [α]_{D}=77.3 (c 1.0, CHCl₃); ¹H NMR (300 MHz, CDCl₃): δ=2.77 (dd, 1H, J=16.1 Hz, J=6.8 Hz), 2.89 (dd, 1H, J=16.1 Hz, J=7.2 Hz), 3.48 (br, OH), 3.85-3.65 (m, 7H), 4.04 (dd, 1H, J=9.8 Hz, J=8.0 Hz), 4.13 (d, 1H, J=9.8 Hz), 4.33 (s, 1H), 4.63 (d, 1H, J=12.5 Hz), 4.68-4.75 (m, 2H), 4.85-4.98 (m, 3H), 5.02 (d, 1H, J=10.9 Hz), 5.08 (d, 1H, J=11.4 Hz), 5.28-5.16 (m, 2H), 5.89 (m, 1H), 7.25-7.49 (m, 20H); ¹³C NMR (75 MHz, CDCl₃): δ=32.0, 52.2, 68.9, 73.5 (2 carbons), 73.6, 75.3, 75.3, 75.6, 78.7, 79.2, 80.6, 84.1, 118.7, 127.7-128.5 (aromatic carbons), 131.7, 138.1, 138.4, 138.4, 138.6, 173.1; Anal. calcd for C₄₀H₄₄O₈: C, 73.60; H, 6.79. Found: C, 73.29; H, 7.04. MS (ES, [M+Na]⁺) calcd for C₄₀H₄₄NaO₈ 675.29, found 675.40.

Methyl (2S)-hydroxy-2-(1-allyl-2,3,4,6-tetra-O-benzyl-α-D-glucopyranosyl)-ethanoate (3-4) [α]_{D}=84.5 (c 1.0, CHCl₃); ¹H NMR (300 MHz, CDCl₃): δ=2.62-2.73 (m, 1H), 2.78-2.90 (dd, 1H, J=15.3 Hz, J=9.4 Hz), 3.59-3.73 (m, 6H), 3.74-3.81 (dd, 1H, J=11.0 Hz, J=3.7 Hz), 3.85 (d, 1H, J=9.6 Hz), 3.99 (d, 1H, J=1.6 Hz), 4.05-4.19 (m, 2H), 4.53 (d, 1H, J=12.3 Hz), 4.62-4.71 (m, 2H), 4.82-4.95 (m, 4H), 5.00 (d, 1H, J=11.0 Hz), 5.10-5.24 (m, 2H), 5.88-6.05 (m, 1H), 7.20-7.40 (m, 20H); ¹³C NMR (75 MHz, CDCl₃): δ=36.6, 52.1, 69.1, 73.0, 73.4, 74.2, 75.1, 75.7, 76.2, 78.4, 78.6, 82.3, 84.0, 119.2, 127.5-128.7 (aromatic carbons), 133.4, 137.3, 138.1, 138.3, 138.5, 170.7; Anal. calcd for C₄₀H₄₄O₈: C, 73.60; H, 6.79. Found: C, 73.41; H, 6.92. MS (ES, [M+Na]⁺); calcd for C₄₀H₄₄NaO₈ 675.29, found 675.40.

Methyl 2-oxo-2-(1-allyl-2,3,4,6-tetra-O-benzyl-β-D-glucopyranosyl)-ethanoate (3-5). Under a nitrogen atmosphere, to a solution of dry dimethyl sulfoxide (133 μL, 1.88 mmol)) in anhydrous dichloromethane (12 mL) cooled below −65° C. with a dry ice-acetone bath, trifluoroacetic anhydride (TFAA, 200 μL, 1.41 mmol) was slowly added with efficient stirring in ca. 10 min. After 10 min below −65° C., a solution of compound 3-3 (307 mg, 0.47 mmol) in dichloromethane (8 mL) was added to the mixture in ca. 15 min. The rate of addition of TFAA or alcohol 3-3 was controlled to keep the temperature below −65° C. The mixture was stirred below −65° C. for 40 min, followed by addition of triethylamine (394 μL, 2.82 mmol) dropwise in ca. 15 min. The reaction was kept below −65° C. for 2 more hours. The cooling bath was then removed and the reaction was allowed to warm up to room temperature, then quenched with H₂O (10 ml) and the aqueous layer was backwashed with dichloromethane (2×15 mL). The combined organic solution was dried with anhydrous Na₂SO₄, concentrated and purified by flash column chromatography (hexanes/ethyl acetate: 6/1) to get 3-5 (296 mg, 97%). [α]_{D}=59.9 (c 1.0, CHCl₃); ¹H NMR (300 MHz, CDCl₃): δ=2.66 (dd, 1H, J=15.6 Hz, J=8.0 Hz), 3.21 (dd, 1H, J=15.6 Hz, J=5.9 Hz), 3.74 (s, 3H), 3.73-3.66 (m, 2H), 3.91-3.76 (m, 3H), 4.24 (d, 1H, J=9.6 Hz), 4.51 (d, 1H, J=11.9 Hz), 4.57 (d, 1H, J=10.3 Hz), 4.61 (d, 1H, J=12.2 Hz), 4.66 (d, 1H, J=10.7 Hz), 4.73 (d, 1H, J=10.3 Hz), 4.83-4.89 (m, 3H), 5.09-5.21 (m, 2H), 5.64 (m, 1H), 7.21-7.40 (m, 20H); ¹³C NMR (75 MHz, CDCl₃): δ=31.2, 52.3, 68.7, 73.4, 73.5, 75.3, 75.6, 75.7, 77.8, 80.3, 83.4, 84.5, 119.2, 127.4-128.5 (aromatic carbons), 130.9, 137.7, 138.0, 138.3, 138.5, 164.9, 195.8; Anal. calcd for C₄₀H₄₂O₈: C, 73.83; H, 6.51. Found: C, 73.43; H, 6.39. MS (ES, [M+Na]⁺); calcd for C₄₀H₄₂NaO₈ 673.28, found 673.39.

Methyl 2-benzylimino-2-(1-allyl-2,3,4,6-tetra-O-benzyl-β-D-glucopyranosyl)-ethanoate (3-6). Under a nitrogen atmosphere, to an ice-cooled solution of 3-5 (296 mg, 0.45 mmol) and benzylamine (148 μL, 1.36 mmol) in anhydrous diethyl ether (15 mL) was added dropwise TiCl₄ (0.23 mL of a 1 M solution in CH₂Cl₂, 0.23 mmol). After complete addition, the ice bath was removed and the reaction mixture stirred for 4 h at room temperature. After this period, the resulting suspension was cooled at 0° C. and poured into 1 M sodium hydroxide solution. The organic layer was separated and the water layer extracted two times with dichloromethane (2×15 mL). The combined organic layer was dried (Na₂SO₄), concentrated and purified by flash column chromatography (hexanes/ethyl acetate: 6/1) to get the mixture of 3-5 and 3-6, which was exposed to the same procedure again, and get 3-6 (323 mg, 96%). [α]_{D}=30.5 (c 1.0, CHCl₃); ¹H NMR (300 MHz, CDCl₃): δ=2.67 (dd, 1H, J=15.6 Hz, J=8.6 Hz), 3.48 (dd, 1H, J=15.6 Hz, J=5.3 Hz), 3.86-3.66 (m, 7H), 3.91 (dd, 1H, J=9.2 Hz, J=8.9 Hz), 4.16 (d, 1H, J=9.2 Hz), 4.43-4.56 (m, 3H), 4.59-5.72 (m, 4H), 4.86-4.93 (m, 3H), 5.03-5.16 (m, 2H), 5.77 (m, 1H), 7.15-7.42 (m, 25H); ¹³C NMR (75 MHz, CDCl₃): δ=32.2, 51.6, 58.7, 69.0, 72.9, 73.4, 75.3, 75.5, 75.8, 78.2, 81.5, 82.4, 84.1, 117.9, 127.7-128.5 (aromatic carbons), 132.5, 138.1, 138.2, 138.4, 138.6, 138.7, 163.7, 165.5; Anal. calcd for C₄₇H₄₉NO₇: C, 76.30; H, 6.68; N, 1.89. Found: C, 75.77; H, 7.05; N, 1.86. MS (ES, [M+Na]⁺) calcd for C₄₇H₄₉NNaO₇ 76234, found 762.38.

Methyl (2S)-benzylamino-2-(1-allyl-(2,3,4,6-tetra-O-benzyl-β-D-glucopyranosyl)-ethanoate (3-7). To an ice-cooled solution of 3-6 (240 mg, 0.32 mmol) in methanol (9 mL) was added NaCNBH₃ (128 mg, 1.95 mmol), followed by 98% AcOH (39 μl, 0.65 mmol). The reaction mixture was stirred for 3 hours at 0° C. and then quenched with water (5 mL) and extracted with CH₂Cl₂ (3×15 mL). The combined organic extracts were dried (Na₂SO₄), concentrated and purified by flash column chromatography (hexanes/ethyl acetate: 5/1) to afford 3-7 (239 mg, quant.). [α]_{D}=32.3 (c 1.0, CHCl₃); ¹H (300 MHz, CDCl₃): δ=2.71 (dd, 1H, J=16.3 Hz, J=6.0 Hz), 2.84 (dd, 1H, J=16.3 Hz, J=7.4 Hz), 3.39 (d, 1H, J=12.8 Hz), 3.47 (s, 1H), 3.59-3.79 (m, 8H), 3.93 (dd, 1H, J=9.5 Hz, J=8.9 Hz), 4.28 (d, 1H, J=9.5 Hz), 4.50 (d, 1H, J=11.6 Hz), 4.58 (d, 1H, J=12.0 Hz), 4.65 (d, 1H, J=10.8 Hz), 4.67 (d, 1H, J=12.0 Hz), 4.80-4.95 (m, 4H), 5.17-5.04 (m, 2H), 5.77 (m, 1H), 7.45-7.10 (m, 26H); ¹³C NMR (75 MHz, CDCl₃): δ=32.4, 51.5, 51.8, 64.8, 69.1, 73.4, 73.5, 74.9, 75.1, 75.7, 78.8, 79.7, 80.2, 84.7, 118.1, 127.7-128.5 (aromatic carbons), 132.0, 138.2, 138.5, 138.7, 138.8, 139.7, 174.0; Anal. calcd for C₄₇H₅₁NO₇: C, 76.09; H, 6.93; N, 1.89. Found: C, 75.84; H, 7.36; N, 2.37. MS (ES, [M+Na]⁺) calcd for C₄₇H₅₁NNaO₇ 764.37, found 764.32.

(1S)-2,3,4,6-Tetra-O-benzyl-1′-N-benzyl-5′(R)-hydroxymethylene-spiro[1,5-anhydro-D-glucitol-1,3′-L-proline methyl ester] (3-8). To a solution of 3-7 (340 mg, 0.46 mmol) in dichloromethane and diethyl ether (12 mL, 1:1) was added iodine (175 mg, 0.69 mmol) at 0° C. The mixture was quenched with saturated sodium thiosulfate solution (5 mL) after overnight. The organic layer was separated and the aqueous layer was backwashed with dichloromethane (2×10 mL), the combined organic solution was dried with anhydrous Na₂SO₄ and concentrated followed by dissolvation in toluene (15 mL) and treated with silver acetate (1.146 g, 6.88 mmol) for overnight at room temperature to get an inseparable mixture of 3-13, 3-14 and 3-15 (323 mg, 93%), which was hydrolyzed with K₂CO₃ (73 mg, 1.3 equiv) in methanol (8 mL) for 1 h at room temperature, and then quenched with saturated ammonium chloride (10 mL) and extracted with CH₂Cl₂ (3×15 mL). The combined organic solution was dried with anhydrous Na₂SO₄, concentrated and purified by flash column chromatography (hexanes/ethyl acetate: from 4/1 to 2/1) to get 3-8 (132 mg, 43%), 3-9 (141 mg, 46%) and 3-10 (20 mg, 6.5%). (3-8) [α]_{D}=47.7 (c 1.0, CHCl₃); ¹H NMR (300 MHz, CDCl₃): δ=2.13 (dd, 1H, J=13.9 Hz, J=5.4 Hz), 2.60 (dd, 1H, J=13.9 Hz, J=11.0 Hz), 3.09 (s, 3H), 3.30 (m, 1H), 3.45-3.85 (m, 11H), 4.02 (d, 1H, J=13.5 Hz), 4.38 (d, 1H, J=12.9 Hz), 4.57-4.71 (m, 4H), 4.83 (d, 1H, J=11.0 Hz), 4.86 (d, 1H, J=11.0 Hz), 5.10 (d, 1H, J=12.7 Hz), 7.10-7.45 (m, 25H); ¹³C NMR (75 MHz, CDCl₃): δ=27.2, 51.8, 59.0, 60.0, 63.0, 69.5, 72.4, 72.7, 73.5, 74.8, 75.1, 75.5, 76.7, 78.8, 86.1, 88.2, 125.8-128.9 (aromatic carbons), 138.0, 138.0, 138.3, 138.8, 138.9, 172.6; HRMS (ES) calcd for C₄₇H₅₂NO₈ [M+H]⁺ 758.3693, found 758.3687.

(1S)-2,3,4,6-Tetra-O-benzyl-1′-N-benzyl-5′(S)-hydroxymethylene-spiro[1,5-anhydro-D-glucitol-1,3′-L-proline methyl ester] (3-9) [α]_{D}=−3.6 (c 1.70, CHCl₃); ¹H NMR (300 MHz, CDCl₃): δ=2.24 (d, 1H, J=14.6 Hz), 2.83 (dd, 1H, J=13.9 Hz, J=10.5 Hz), 3.05-3.23 (br, OH), 3.31 (s, 3H), 3.43 (d, 1H, J=11.1 Hz), 3.62-3.9 (m, 10H), 4.00 (d, 1H, J=14.6 Hz), 4.47 (d, 1H, J=12.5 Hz), 4.54-4.77 (m, 4H), 4.82-4.94 (m, 2H), 5.24 (d, 1H, J=11.8 Hz), 7.12-7.44 (m, 25H); ¹³C NMR (75 MHz, CDCl₃): δ=27.9, 51.1, 51.7, 61.3, 62.6, 69.1, 72.5, 72.9, 73.0, 73.7, 74.9, 75.7, 77.0, 78.5, 85.7, 86.2, 126.0-128.6 (aromatic carbons), 138.0, 138.2, 138.4, 138.8, 138.9, 170.5; HRMS (ES) calcd for C₄₇H₅₂NO₈ [M+H]⁺ 758.3693, found 758.3696.

(1S)-2,3,4,6-Tetra-O-benzyl-1′-N-benzyl-5′(S)-hydroxy-spiro[1,5-anhydro-D-glucitol-1,3′-L-pipecolic methyl ester] (3-10) [α]_{D}=34.7 (c 1.0, CHCl₃); ¹H NMR (300 MHz, CDCl₃): δ=2.25-2.36 (m, 2H), 2.76 (dd, 1H, J=11.0 Hz, J=5.2 Hz), 3.04 (dd, 1H, J=10.6 Hz, J=9.6 Hz), 3.32 (s, 3H), 3.42 (s, 1H), 3.53-3.82 (m, 7H), 3.90-4.07 (m, 2H), 4.58-4.73 (m, 5H), 4.82 (d, 1H, J=11.2 Hz), 4.87 (d, 1H, J=10.9 Hz), 5.10 (d, 1H, J=11.7 Hz), 7.12-7.39 (m, 25H); ¹³C NMR (75 MHz, CDCl₃) δ=29.7, 30.7, 51.0, 54.5, 59.0, 63.5, 69.5, 69.9, 72.4, 73.3, 73.9, 74.9, 75.4, 78.1, 79.1, 80.4, 84.6, 126.3-128.6 (aromatic carbons), 138.1, 138.2, 138.3, 138.6, 138.7, 170.7; HRMS (ES) calcd for C₄₇H₅₂NO₈ [M+H]⁺758.3693, found 758.3686.

(1S)-5′(R)-hydroxymethylene-spiro[1,5-anhydro-D-glucitol-1,3′-L-proline methyl ester] (3-11). Under the nitrogen atmosphere, to the solution of compound 3-8 (200 mg, 0.25 mmol) in methanol (10 mL) was added the 1 M hydrochloride acid solution (0.38 mL, 0.38 mmol) and the palladium hydroxide (20 wt % Pd on carbon, 50 mg). The mixture was exposed to hydrogen condition (H₂, 10 psi) and stirred for 6 hours. The solution was filtrated and evaporated in vacuum to get the product 3-11 (75 mg, quant.) [α]_{D}=64.1 (c 1.0, MeOH); ¹H NMR (300 MHz, CD₃OD): δ=2.13 (dd, 1H, J=15.0 Hz, J=2.8 Hz), 2.41 (dd, 1H, J=15.0 Hz, J=10.8 Hz), 3.23-3.21 (m, 5H), 3.49 (m, 1H), 3.6-3.71 (m, 2H), 3.85-4.00 (m, 4H), 4.22 (s, 1H); ¹³C NMR (75 MHz, CD₃OD): δ=27.3, 54.3, 61.5, 61.9, 63.0, 68.7, 70.8, 71.6, 76.6, 77.0, 88.3, 168.5; HRMS (ES) calcd for C₁₂H₂₂NO₈ [M+H]⁺ 308.1340, found 308.1343.

(1S)-5′(S)-hydroxymethylene-spiro[1,5-anhydro-D-glucitol-1,3′-L-proline methyl ester] (3-12) (The detailed procedure was the same as 3-11.) [α]_{D}=75.7 (c 1.10, MeOH); ¹H NMR (300 MHz, CD₃OD) δ=2.09 (m, 1H), 2.56 (dd, 1H, J=10.3 Hz, J=13.6 Hz), 3.24 (m, 1H), 3.30-3.40 (m, br, 1H, overlapping with solvent peak), 3.47 (m, 1H), 3.57-3.68 (m, 2H), 3.72-3.95 (m, 6H), 4.18-4.33 (m, 2H); ¹³C NMR (75 MHz, CD₃OD): δ=27.8, 54.2, 62.6, 63.1, 63.2, 69.1, 71.2, 71.7, 76.7, 76.9, 88.2, 167.9; HRMS (ES) calcd for C₁₂H₂₂NO₈ [M+H]⁺ 308.1340, found 308.1348.

(1S)-2,3,4,6-Tetra-O-benzyl-1′-N-benzyl-5′(R)-methylenehydroxy acetate-spiro[1,5-anhydro-D-glucitol-1,3′-L-proline methyl ester] (3-13). To a solution of 3-8 (60 mg, 0.079 mmol) in pyridine (1 mL) was added acetic anhydride (37 μL, 0.395 mmol) and stirred for 5 hours. The pyridine was removed with high vacuum. The crude product was purified by flash column chromatography (hexanes/ethyl acetate: 4/1) to get 3-13 (62 mg, quant.). [α]_{D}=50.3 (c 1.0, CHCl₃); ¹H NMR (300 MHz, CDCl₃): δ=2.05 (s, 3H), 2.18 (dd, 1H, J=13.0 Hz, J=10.8 Hz), 2.33 (dd, 1H, J=13.6 Hz, J=5.7 Hz), 3.13 (s, 3H), 3.32 (m, 1H), 3.53 (s, 1H), 3.63-3.78 (m, 6H), 3.83 (d, 1H, J=14.4 Hz), 4.08 (d, 1H, J=13.7 Hz), 4.24 (d, 2H, J=6.0 Hz), 4.42 (d, 1H, J=12.3 Hz), 4.58-4.70 (m, 4H), 4.83 (d, 1H, J=9.9 Hz), 4.86 (d, 1H, J=9.9 Hz), 5.06 (d, 1H, J=12.3 Hz), 7.13-7.43 (m, 25H); ¹³C NMR (75 MHz, CDCl₃): δ=20.9, 30.0, 51.5, 60.4, 60.8, 67.2, 69.4, 72.5, 72.8, 73.5, 75.1, 75.5, 76.0, 76.7, 78.7, 86.1, 87.5, 126.0-128.8 (aromatic carbons), 138.03, 138.04, 138.4, 138.9, 139.3, 171.0, 172.0; HRMS (ES) calcd for C₄₉H₅₄NO₉ [M+H]⁺ 800.3793, found 800.3794.

(1S)-2,3,4,6-Tetra-O-benzyl-1′-N-benzyl-5′(S)-methylenehydroxyl acetate-spiro[1,5-anhydro-D-glucitol-1,3′-L-proline methyl ester] (3-14) (The detailed procedure was the same as 3-13.) [α]_{D}=6.2 (c 1.0, CHCl₃); ¹H NMR (300 MHz, CDCl₃): δ=2.01 (s, 3H), 2.14 (dd, 1H, J=15.4 Hz, J=1.1 Hz), 2.82 (dd, 1H, J=13.9 Hz, J=10.0 Hz), 3.26 (s, 3H), 3.58-3.82 (m, 9H), 3.59-3.82 (m, 2H), 4.21 (dd, 1H, J=10.7 Hz, J=5.0 Hz), 4.43 (d, 1H, J=12.6 Hz), 4.57-4.75 (m, 4H), 4.82 (d, 1H, J=11.2 Hz), 4.86 (d, 1H, J=11.2 Hz), 5.15 (d, 1H, J=12.3 Hz), 7.10-7.40 (m, 25H); ¹³C NMR (75 MHz, CDCl₃): δ=21.0, 27.7, 51.1, 53.0, 60.0, 67.2, 69.0, 72.9, 73.0, 73.2, 73.7, 75.1, 75.6 (2 carbons), 78.7, 85.9, 86.9, 126.0-128.5 (aromatic carbons), 137.0 (2 carbons), 138.5, 138.9, 139.5, 170.9, 170.9; HRMS (ES) calcd for C₄₉H₅₄NO₉ [M+H]⁺ 800.3793, found 800.3793.

(1S)-2,3,4,6-Tetra-O-benzyl-1′-N-benzyl-5′(S)-O-acetyl-spiro[1,5-anhydro-D-glucitol-1,3′-L-pipecolic methyl ester] (3-15) (The detailed procedure was the same as 3-13.) [α]_{D}=44.0 (c 1.0, CHCl₃); ¹H NMR (300 MHz, CDCl₃): δ=2.05 (s, 3H), 2.32 (dd, 1H, J=13.6 Hz, J=11.9 Hz), 2.52 (dd, 1H, J=13.7 Hz, J=4.0 Hz), 2.90 (dd, 1H, J=10.4 Hz, J=5.6 Hz), 3.24 (dd, 1H, J=10.4 Hz, J=10.8 Hz), 3.29 (s, 3H), 3.46-3.99 (m, 9H), 4.45 (d, 1H, J=12.9 Hz), 4.59-4.93 (m, 6H), 5.09 (m, 1H), 5.15 (d, 1H, J=12.4 Hz), 7.10-7.44 (m, 25H); ¹³C NMR (75 MHz, CDCl₃): δ=21.6, 26.4, 50.2, 50.4, 59.5, 69.7, 72.8, 73.4, 74.4 (2 carbons), 75.4 (2 carbons), 75.8, 78.7, 79.4, 80.9, 85.3, 126.50-128.90 (aromatic carbons), 138.8 (3 carbons), 139.2, 139.4, 170.7 (2 carbons); HRMS (ES) calcd for C₄₉H₅₄NO₉ [M+H]⁺800.3793, found 800.3788.

(1S)-1′-N-tert-butoxycarbonyl-5′(R)-methylenehydroxy acetate-spiro[1,5-anhydro-D-glucitol-1,3′-L-proline methyl ester] (3-16). To a mixture of 3-11 (100 mg, 0.13 mmol) and Pd(OH)₂ (40 mg, 20% wt on charcoal) in methanol (10 mL) was added the solution of hydrochloride (250 μL of 1M HCl solution, 0.25 mmol) and stirred under H₂ (15 psi) for 8 hours at room temperature. The catalyst was removed by filtration and the solvent was removed under the vacuum. The unprotected product was treated with triethylamine (53 μL, 0.38 mmol) and di-tent-butyl dicarbonate (56 mg, 0.25 mmol) in methanol (2 mL) for 1 hour at room temperature. The solvent was removed under vacuum. The crude product was purified by flash column chromatography (CH₂Cl₂/MeOH: 7/1) to get 3-16 (51 mg, 90%). [α]_{D}=68.4 (c 1.3, MeOH); ¹H NMR (300 MHz, CD₃OD, two isomers): δ=1.45 (s, 9H), 2.09 (s, 3H), 2.13-2.44 (m, 2H), 3.27-3.46 (m, 3H, partially overlapping with methanol peaks), 3.57-3.86 (m, 6H), 4.06 (m, 1H), 4.23 (s, 1H), 4.31-4.45 (m, 1H), 4.53-4.67 (m, 1H); ¹³C NMR (75 MHz, CD₃OD, two isomers): δ=20.9 (2 carbons), 28.56/28.63 (6 carbons), 29.2/29.7, 52.9 (2 carbons), 56.2/56.3, 62.8 (2 carbons), 66.0/66.4, 71.2 (2 carbons), 71.4/71.8, 71.5 (2 carbons), 75.9/76.0, 77.3 (2 carbons), 81.8/82.3, 86.2/86.7, 155.6/156.2, 172.0/171.8, 172.7/172.8; HRMS (ES) calcd for C₁₉H₃₁NNaO₁ [M+Na]⁺ 472.1795, found 472.1783.

(1S)-1′-N-tent-butoxycarbonyl-5′(S)-methylenehydroxy acetate-spiro[1,5-anhydro-D-glucitol-1,3′-L-proline methyl ester] (3-17) (The detailed procedure was the same as 3-16.). [α]_{D}=15.7 (c 1.35, MeOH); ¹H NMR (300 MHz, CD₃OD, two isomers): δ=1.43 (s, 9H), 2.09 (s, 3H), 2.14-2.42 (m, 2H), 3.24-3.50 (m, 3H, partially overlapping with methanol peaks), 3.50-3.87 (m, 6H), 4.04-4.63 (m, 4H); ¹³C NMR (75 MHz, CD₃OD, two isomers): δ=20.8 (2 carbons), 25.5/26.1, 28.5/28.7 (6 carbons), 53.0 (2 carbons), 57.2/57.3, 62.6/62.8, 65.8/65.9, 71.2 (2 carbons), 71.35 (2 carbons), 71.8/71.4, 75.8/75.9, 77.0/77.1, 81.9/82.3, 88.4/87.4, 155.8/156.1, 172.1/171.6, 172.6/172.4; HRMS (ES) calcd for C₁₉H₃₁NNaO₁₁ [M+Na]⁺472.1795, found 472.1788.

(1S)-6-Azido-6-deoxy-1′-N-tert-butoxycarbonyl-5′(R)-methylenehydroxy acetate-spiro[1,5-anhydro-D-glucitol-1,3′-L-proline methyl ester] (3-18). To a solution of compound 16 (40 mg, 0.09 mmol) in pyridine (1 mL) was added p-toluenesulfonyl chloride (42 mg, 0.22 mmol) and stirred for 12 hours at room temperature. The mixture was concentrated and purified by flash column chromatography (CH₂Cl₂/MeOH: 10/1) to provide tosyl ester, which was treated with sodium azide (116 mg, 1.8 mmol) in DMF (1.5 mL) and stirred at 80° C. for 12 hours. The mixture was filtered, concentrated and purified by flash column chromatography (CH₂Cl₂/MeOH: 15/1) to get 3-18 (38 mg, 92%). [α]_{D}=31.5 (c 1.35, MeOH); ¹H NMR (300 MHz, CD₃OD, two isomers): δ=1.44 (s, 9H), 2.10 (s, 3H), 2.14-2.48 (m, 2H), 3.18-3.49 (m, 4H, partially overlapping with methanol peaks), 3.58-3.77 (m, 5H), 4.01-4.17 (m, 1H), 4.21 (s, 1H), 4.32-4.48 (m, 1H), 4.52-4.64 (m, 1H); ¹³C NMR (75 MHz, CD₃OD, two isomers): δ=20.9 (2 carbons), 28.6 (6 carbons,), 29.0/29.6, 52.6/52.8, 53.0 (2 carbons), 56.2/56.4, 66.2/66.4, 71.1 (2 carbons), 71.4/71.9, 72.4/72.8, 75.2/75.4, 77.0/77.1, 81.8/82.3, 86.4/87.0, 155.8/156.3, 171.6/171.7, 172.7/172.8; HRMS (ES) calcd for C₁₉H₃₀N₄NaO₁₀ [M+Na]⁺497.1860, found 497.1849.

(1S)-6-Azido-6-deoxy-3′-N-tert-butoxycarbonyl-5′(S)-methylenehydroxy acetate spiro[1,5-anhydro-D-glucitol-1,3′-L-proline methyl ester] (3-19) (The detailed procedure was the same as 3-18.). [α]_{D}=29.1 (c 0.7, MeOH); ¹H NMR (300 MHz, CD₃OD, two isomers): δ=1.45 (s, 9H), 2.09 (s, 3H), 2.14-2.48 (m, 2H), 3.23-3.44 (m, 3H, partially overlapping with methanol peaks), 3.50-3.78 (m, 6H), 4.06-4.34 (m, 3H), 4.39-4.63 (m, 1H); ¹³C NMR (75 MHz, CD₃OD, two isomers): δ=20.8 (2 carbons), 25.6/26.2, 28.5/28.6 (6 carbons,), 53.0 (2 carbons), 53.0/53.1, 57.2 (2 carbons), 65.7/65.9, 71.1 (2 carbons), 71.9/71.5, 72.2/72.3, 74.7/74.8, 76.7/76.8, 82.0/82.4, 88.8/87.8, 155.8/156.1, 171.9/171.4, 172.6/172.4; HRMS (ES) calcd for C₁₉H₃₀N₄NaO₁₀ [M+Na]⁺497.1860, found 497.1852.

(1S)-6-Azido-6-deoxy-1′-N-acetyl-5′(R)-hydroxymethylene-spiro[1,5-anhydro-D-glucitol-1,3′-L-proline methyl ester] (3-20). The compound 3-18 (30 mg, 0.063 mmol) was dissolved in a mixture of dichloromethane and trifluoroacetic acid (1.5 mL/0.5 mL) and stirred for 1 hour at room temperature. The solution was concentrated at vacuum and then treated with a mixture of pyridine and acetic acid (1 mL/1 mL) and stirred for 12 hours at room temperature and then concentrated at vacuum. After that, it was dissolved in a solution of sodium methoxide in methanol (0.1 M, 2 mL) and stirred for 4 hours at room temperature followed by the neutralization with Amberlite IRC-50S ion-exchange resin (H⁺). The mixture was filtered and filtrate was concentrated and purified by the flash column chromatography (ethyl acetate/methanol:6/1) to get compound 3-20 (22 mg, 96%). [α]_{D}=19.2 (c 0.75, MeOH); ¹H NMR (300 MHz, CD₃OD, two isomers): δ=1.82 (s, 1.74H), 2.04 (s, 1.26H), 2.05-2.49 (m, 2H), 3.06-3.34 (m, 4H, partially overlapping with methanol peaks), 3.49-3.94 (m, 7H), 4.04-4.16 (m, 1H), 4.20 (s, 0.58H), 4.42 (s, 0.42H); ¹³C NMR (75 MHz, CD₃OD, two isomers): δ=22.8/21.6, 27.7/29.7, 52.8/52.7, 53.6/53.2, 60.6/60.1, 64.9/65.6, 71.1 (2 carbons), 72.6/71.1, 72.6/72.8, 75.4/75.7, 76.9/77.0, 87.5/86.4, 171.6/171.9, 173.6 (2 carbons); HRMS (ES) calcd for C₁₄H₂₂N₄NaO₈ [M+Na]⁺397.1335, found 397.1351.

(1S)-6-Azido-6-deoxy-1′-N-acetyl-5′(S)-hydroxymethylene-spiro[1,5-anhydro-D glucitol-1,3′-L-proline methyl ester] (3-21) (The detailed procedure was the same as 3-20.). [α]_{D}=46.5 (c 0.55, MeOH); ¹H NMR (300 MHz, CD₃OD, two isomers): δ=1.77 (s, 1.09H), 2.08 (s, 1.91H), 2.15-2.41 (m, 2H), 3.23-3.68 (m, 10H, partially overlapping with methanol peaks), 3.79 (dd, 0.64H, J=5.28 Hz, J=10.95 Hz), 3.96-4.23 (m, 2.36H); ¹³C NMR (75 MHz, CD₃OD, two isomers): δ=22.04 (22.48), 26.54 (25.35), 52.97 (53.14), 53.13 (53.46), 61.52 (61.38), 64.76 (63.61), 71.14 (71.19), 72.03 (72.44), 72.27 (73.01), 74.68 (74.73), 76.71 (76.62), 87.40 (88.99), 170.78 (171.20), 173.56 (173.50); HRMS (ES) calcd for C₁₄H₂₂N₄NaO₈ [M+Na]⁺397.1335, found 397.1348.

(1S)-6-Amino-6-deoxy-1′-N-acetyl-5′(R)-hydroxymethylene-spiro[1,5-anhydro-D-glucitol-1,3′-L-proline methyl ester] HCl salt (3-22). To a solution of 3-20 (20 mg, 0.053 mmol) and Pd(OH)₂ (20 mg, 20% wt on charcoal) in methanol (5 mL) was added the solution of hydrochloride (800 μL of 1M HCl solution, 0.08 mmol) and stirred under H₂ (15 psi) for 20 minutes at room temperature. The catalyst was removed by the regular filtration and the solvent was removed under the vacuum to afford pure product 3-22 (20 mg, quant.). [α]_{D}=65.2 (c 0.5, MeOH); ¹H NMR (500 MHz, D₂O, two isomers): δ=1.86 (s, cis, 1.68H), 1.98 (dd, cis, 0.56H, J=10.39 Hz, J=14.57 Hz), 2.04-2.09 (m, trans, 0.44H+1.32H), 2.36 (dd, cis, 0.56H, J=6.76 Hz, J=14.56 Hz), 2.47 (dd, trans, 0.44H, J=7.42 Hz, J=14.62 Hz), 2.98 (m, 1H), 3.19-3.37 (m, 3H), 3.59-3.67 (m, 5H), 3.68-3.74 (m, 1H), 3.81-3.88 (m, 1H), 4.00-4.07 (m, cis, 0.56H), 4.11-4.18 (m, trans, 0.44H). 4.42 (s, cis, 0.56H), 4.44 (s, trans, 0.44H); ¹³C NMR (75 MHz, D₂O): cis, δ=22.2, 26.7, 40.5, 53.5, 58.5, 62.4, 69.2, 69.7, 70.7, 71.2, 74.4, 86.2, 171.4, 174.4; trans, δ=20.9, 28.8, 40.4, 53.2, 58.4, 63.5, 69.2, 69.7, 69.8, 70.9, 74.4, 85.1, 171.4, 174.5; HRMS (ES) calcd for C₁₄H₂₅N₂O₈ [M+H]⁺ 349.1611, found 349.1623.

(1S)-6-Amino-6-deoxy-1′-N-acetyl-5′(S)-hydroxymethylene-spiro[1,5-anhydro-D-glucitol-1,3′-L-proline methyl ester] HCl salt (3-23). (The detailed procedure was the same as 3-22.). [α]_{D}=24.3 (c 0.55, MeOH); ¹H NMR (500 MHz, D₂O, two isomers): δ=1.93 (s, cis, 0.60H), 2.21 (s, trans, 2.40H), 2.29-2.46 (m, 2H), 3.20-3.59 (m, 5H), 3.63-3.82 (m, 5H), 3.85-3.94 (m, 1H), 4.33-4.41 (m, 1H), 4.43 (s, trans, 0.8H), 4.62 (s, 0.2H); ¹³C NMR (75 MHz, D₂O): cis, δ=21.8, 25.0, 40.2, 53.6, 59.5, 62.3, 69.3, 69.6, 70.5, 71.2, 74.2, 87.6, 171.4, 174.4; trans, δ=21.3, 25.9, 39.9, 53.2, 59.7, 63.2, 69.3, 69.5, 70.3 (2 carbons), 74.2, 86.3, 170.8, 174.5; HRMS (ES) calcd for C₁₄H₂₅N₂O₈ [M+H]⁺ 349.1611, found 349.1618.

(1S)-6-Amino-6-deoxy-1′-N-acetyl-5′(R)-hydroxymethylene-spiro[1,5-anhydro-D-glucitol-1,3′-L-proline methyl amide] HCl salt (3-24). To a solution of methylamine in ethanol (37% wt, 1 mL) was added compound 3-20 (15 mg, 0.04 mmol) and stirred for 18 hours at room temperature. The mixture was concentrated and purified by flash column chromatography (dichloromethane/methanol:2/1) to quantitatively afford C-terminal methyl amide intermediate, which was dissolved in a solution of Pd(OH)₂ (15 mg, 20% wt on charcoal) and 1 M hydrochloride acid solution (80 μL, 0.08 mmole). The mixture was stirred under H₂ (15 psi) for 20 minutes at room temperature The catalyst was removed by the regular filtration and the solvent was removed under the vacuum to afford pure product 3-24 (14 mg, 93%). [α]_{D}=54.8 (c 0.35, MeOH); ¹H NMR (500 MHz, D₂O, two isomers): δ=2.02 (s, cis, 2.24H), 2.21 (s, trans, 0.76H), 2.27 (dd, cis, 0.75H, J=11.31 Hz, J=14.27 Hz), 2.35-2.44 (m, 1H), 2.53 (dd, trans, 0.25H, J=6.17 Hz, J=14.11 Hz), 2.67-2.83 (m, 3.75H) 2.98-3.05 (m, 0.75H), 3.25 (dd, 0.25H, J=11.31 Hz, J=14.27 Hz), 3.30-3.39 (m, 1.25H), 3.41-3.56 (m, 2H), 3.68-3.77 (m, 1.75H). 3.83 (dd, trans, 0.25H, J=1.81 Hz, J=12.43 Hz), 4.09-4.36 (m, 3H); ¹³C NMR (75 MHz, D₂O): cis, δ=27.2, 30.1, 30.8, 46.5, 62.9 (2 carbons), 64.5, 74.6, 76.0, 77.4, 80.1, 90.3, 176.5, 179.6; trans, 25.9, 30.8, 32.1, 46.4, 62.8, 63.0, 66.0, 74.5, 75.9, 76.5, 78.4, 89.0, 176.6, 179.6; HRMS (ES) calcd for C₁₄H₂₆N₃O₇ [M+H]⁺ 348.1771, found 348.1759.

(1S)-6-Amino-6-deoxy-1′-N-acetyl-5′(S)-hydroxymethylene-spiro[1,5-anhydro-D-glucitol-1,3′-L-proline methyl amide] HCl salt (3-25) (The detailed procedure was the same as 3-24.). [α]_{D}=19.2 (c 0.40, MeOH); ¹H NMR (500 MHz, D₂O, two isomers): δ=1.80 (s, cis, 1.08H), 2.08 (s, trans, 1.92H), 2.16-2.32 (m, 2H), 2.56 (s, trans, 1.92H), 2.62 (s, cis, 1.08H), 3.09-3.54 (m, 6H), 3.62-3.68 (m, 1H), 3.76 (dd, trans, 0.64H, J=5.26 Hz, J=11.50 Hz), 3.83 (dd, cis, 0.36H, J=4.60 Hz, J=11.04 Hz), 4.19 (s, trans, 0.64H), 4.21-4.30 (m, 1.36H); ¹³C NMR (75 MHz, D₂O): cis, δ=26.7, 30.1, 31.2, 45.1, 64.6, 67.3, 74.3, 74.6, 75.5, 77.4, 79.4, 92.6, 175.5, 179.2; trans, δ=26.5, 31.0 (2 carbons), 44.9, 64.7, 68.2, 74.3, 74.5, 75.2, 76.6, 79.4, 91.4, 175.0, 179.1; HRMS (ES) calcd for C₁₄H₂₆N₃O₇ [M+H]⁺ 348.1771, found 348.1766.

Measurement of equilibrium constant: The calculation was based on the integration of well-resolved peaks of the γ-protons, N-terminal methyl group and α-proton in ¹H NMR.

Temperature coefficient (Δδ/ΔT) experiments: 1 D ¹H-NMR spectroscopy of 16 mM solutions of 3-24 and 3-25 in 100.0% Me₂SO-d₆ were recorded on Bruker AMX500 at 25° C., and from 25 to 44° C. with increments of 5° C., using routine techniques. Chemical shift (6) of hydroxyl and amino groups are expressed in ppm and calibrated with respect to the residual DMSO signal (1H, 2.49 ppm). The chemical shift changes (Δδ) at different temperatures were calculated with respect to the chemical shift of hydroxyl and amino groups at 25° C.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

### Example 1

1. (a) Brandits, J. F.; Halvorson, H. R.; Brennan, M. Biochemistry 1975, 14, 4953. (b) Schmid, F. X.; Baldwin, R. L. Proc. Nat. Acad. Sci. U.S.A. 1978, 75, 4764. (c) Hurle, M. R.; Marks, C. B.; Kosen, P. A.; Anderson, S.; Kuntz, I. D. Biochemistry 1990, 29, 4410. (d) Jackson, S. E.; Fersht, A. R. Biochemistry 1991, 30, 10436.
2. (a) Stryer, L. Biochemistry, 4^{th} ed.; W.H. Freeman and Company: New York, 1999. (b) Kakinoki,
3. S.; Hirano, Y.; Oka, M. Polym. Bull. (Berlin) 2005, 53, 109.
4. Reddy, K. V. R.; Yedery, R. D.; Aranha, C. Int. J. Antimicrobial Agents 2004, 24, 536.
5. Buku, A.; Faulstich, H.; Wieland, T.; Dabrowski, J. Proc. Natl. Acad. Sci. USA. 1980, 77, 2370.
6. Nakajima, T.; Volcani, B. E. Science 1969, 164, 1400.
7. Taylor, S. W.; Waite, J. H.; Ross, M. M.; Shabanowitz, J.; Hunt, D. F. J. Am. Chem. Soc. 1994, 116, 10803.
8. (a) Berg, R. A.; Prockop, D. J. Biochem. Biophys. Res. Commun. 1973, 52, 115. (b) Vitagliano, L.; Berisio, R.; Mazzarella, L.; Zagari, A. Biopolymers 2001, 58, 459; And references therein. (c) Holmgren, S. K.; Taylor, K. M.; Bretscher, L. E.; Raines, R. T. Nature 1998, 392, 666. (d) Bretscher, L. E.; Jenkins, C. L.; Taylor, K. M.; deRider, M. L.; Raines, R. T. J. Am. Chem. Soc. 2001, 123, 777. (e) Jenkins, C. L.; Raines, R. T. Nat. Prod. Rep. 2002, 19, 49.
9. Pearce, G.; Ryan, C. A. J. Biol. Chem. 2003, 278, 30044; And references therein.
10. Beausoleil, E.; Lubell, W. D. J. Am. Chem. Soc. 1996, 118, 12902. And references therein.
11. Delaney, N. G.; Madison, V. J. J. Am. Chem. Soc. 1982, 104, 6635.
12. Samanen, J.; Zuber, G.; Bean, J.; Eggleston, D.; Romoff, T.; Kopple, K.; Saunders, M.; Regoli, D. Int. J. Pept. Protein. Res. 1990, 35, 501.
13. Quancard, J.; Labonne, A.; Jacquot, Y.; Chassaing, G.; Lavielle, S.; Karoyan, P. J. Org. Chem. 2004, 69, 7940.
14. Che, Y.; Marshall, G. R. J. Org. Chem. 2004, 69, 9030. And references therein.
15. Tam, J. P.; Miao, Z. J. Am. Chem. Soc. 1999, 121, 9013. And references therein.
16. Cavelier, F.; Vivet, B.; Martinez. J.; Aubry, A.; Didierjean, C.; Vicherat, A.; Marraud, M. J. Am. Chem. Soc. 2002, 124, 2917. And references therein.
17. Sharma, R.; Lubell, W. D. J. Org. Chem. 1996, 61, 202. And references therein.
18. Jeannotte, G.; Lubell, W. D. J. Org. Chem. 2004, 69, 4656.
19. (a) Wagaw, S.; Rennels, R. A.; Buchwald, S. L. J. Am. Chem. Soc. 1997, 119, 8451; (b) Kuwano, R.; Sato, K.; Kurokawa, T.; Karube, D.; Ito, Y. J. Am. Chem. Soc. 2000, 122, 7614; (c) Viswanathan, R.; Prabhakaran, E. N.; Plotkin, M. A.; Johnston, J. N. J. Am. Chem. Soc. 2003, 125, 163.
20. Koep, S.; Gais, H-J.; Raabe, R. J. Am. Chem. Soc. 2003, 125, 13243. And references therein.
21. Owens, N.; Braun, C.; Schweizer, F. J. Org. Chem., 2007, 72, 4635.
22. For selected examples of bicyclic proline analogues incorporated into bioactive peptides see: (a) Cluzeau, J.; Lubell, W. D. Biopolymers, Peptide Science 2005, 80, 98. (b) Blankley, C. J.; Kaltenbronn, J. S.; DeJohn, D. E.; The inventorsner, A.; Bennett, L. R.; Bobowski, G.; Krolls, U.; Johnson, D. R.; Pearlman, W. M.; Hoefle, M. L. J. Med. Chem. 1987, 30, 992. (c) Dumy, P.; Keller, M; Ryan, D. E.; Rohwedder, B.; Wöhr, T.; Mutter, M. J. Am. Chem. Soc. 1997, 119, 918. (d) Li. W.; Moeller, K. D. J. Am. Chem. Soc. 1996, 118, 10106.
23. Keller, M.; Sager, C.; Dumy, P.; Schutkowski, M.; Fisher, G. S.; Mutter, M. J. Am. Chem. Soc. 1998, 120, 2714. And references therein.
24. Wöhr, T.; Wahl, F.; Nefzi, A.; Rohwedder, B.; Sato, T.; Sun, X.; Mutter, M. J. Am. Chem. Soc. 1996, 118, 9218.
25. Zhang, K.; Schweizer, F. Synlett 2005, 3111.
26. Gruner, S. W.; Locardi, E.; Lohof, E.; Kessler, H. Chem. Rev. 2002, 102, 491.
27. Jenkins, C. L.; Bretscher, L. E.; Guzei, I. A.; Raines, R. T. J. Am. Chem. Soc. 2003, 125, 6422.
28. (a) Cox, C.; Lectka, T. J. Am. Chem. Soc. 1998, 120, 10660. (b) Mizushima, S.; Shimanouchi, T.; Tsuboi, M.; Sugita, T.; Kurosaki, K.; Mataga, N.; Souda, R. J. Am. Chem. Soc. 1952, 74, 4639. (c) Liang, G. B.; Rito, C. J.; Gellman, S. H. Biopolymers 1992; 32, 507. A 40 ms Gaussian pulse with a 560 ms mixing time was used.
29. Taylor, C. M.; Hardre, R.; Edwards, P. J. B.; Park, J. H. Org. Lett. 2003, 23, 4413. and references therein.
30. (a) DeRider, M. L.; Wilkens, S. J.; Waddell, M. J.; Bretscher, L. E.; The inventorsinhold, F.; Raines, R. T.; Markley, J. L. J. Am. Chem. Soc. 2002, 124, 2497. (b) Hinderaker, M. P.; Raines, R. T. Protein Sci. 2003, 12, 1188. (c) Hodges, J. A.; Raines, R. T. Org. Lett. 2006, 8, 4695.
31. Delaney, N. G.; Madison, V. Int. J. Peptide Protein Res. 1982, 19, 543.
32. (a) Perrin, C. L.; Dwyer, T. J. Chem. Rev. 1990, 90, 935. (b) Reimer, U; Scherer, G; Drewello, M; Kruber, S; Schutkowski, M; Fischer, G. J. Mol. Biol. 1998, 279, 449.
33. Stein, R. L. Adv. Protein Chem. 1993, 44, 1.
34. Eyring, H. J. Chem. Phys. 1935, 3, 107.
35. Jackson, M.; Mantsch, H. H. Crit. Rev. Biochem. Molec. Biol. 1995, 30, 95.
36. Eberhardt, E. S.; Panasik, Jr. N.; Raines, R. T. J. Am. Chem. Soc. 1996, 118, 12261.
37.(a) Leeflang, B. R.; Vliegenthart, J. F. G.; Kroon-Batenburg, L. M. J.; Eijck, B. P.; Kroon, J. Carbohydr. Res. 1992, 230, 41. (b) St.-Jacques, M.; Sundarajan, P. R.; Taylor, K. J.; Marchessault, R. H. J. Am. Chem. Soc. 1976, 98, 4386.
38. Koch, W.; Holthausen, M. C. A Chemist's Guide to Density Functional Theory. 2^{nd} ed., Willey, The inventorsinheim, 2000.
39. Cramer, C. J. Essentials of Computational Chemistry: Theories and Models, 2^{nd} ed.; Wiley, New York, 2004.
40. Becke, A. D., J. Chem. Phys. 1993, 98, 5684.
41. Lee, C.; Yang, W.; Parr, R. G. Phys. Rev. B: Codens. Matter 1988, 37, 785.
42. Stephens, P. J.; Devlin, F. J.; Chabalowski, C. F.; Frisch, M. J. J. Phys. Chem. 1994, 98, 11623.
43. Hehre, W. J. A Guide to Molecular Mechanics and Quantum Chemical Calculations, Wavefunction Inc.: Irvine, Calif., 2003, pp. 393.
44. Tomasi, J.; Mennucci, B.; Cammi, R. Chem. Rev., 2005. 105, 2999. Backbone torsion angles: ψ′=C—C′—N—C^{α}, φ=C′—N—C^{α}—C′, ψ=N—C^{α}—C′—O, ω=C^{α}—C′—O—C. Endocyclic torsion angles: χ⁰=C^{δ}—N—C^{α}—C^{β}, χ¹=N—C^{α}—C^{β}—C^{γ}, χ²=C^{α}—C^{β}—C^{γ}—C^{δ}, χ³=
45. C^{β}—C^{γ}—C^{δ}—N, χ⁴=C^{γ}—C^{δ}—N—C^{α}.
46. Song, Il. K.; Kang, Y. K. J. Phys. Chem. B, 2005, 109, 16982.
47. Taylor, C. M.; Hardre, R.; Edwards, P. J. B. J. Org. Chem. 2005, 70, 1306.
48. Morozov, A.; Kortemme, T.; Tsemekhman, K.; Baker, D. Proc. Natl. Acad. Sci. USA 2004. 101, 6946.
49. Koskinen, A. M. P.; Helaja, J.; Kumpulainen, E. T. T.; Koivisto, J.; Mansikkamäki, H.; Rissanen, K. J. Org. Chem. 2005. 70, 6447.
50.(a) Eberhardt, E. S.; Panasik, Jr. N.; Raines, R. T. J. Am. Chem. Soc. 1996, 118, 12261. (b) Renner, C.; Alefelder, S.; Bae, J. H.; Budisa, N.; Huber, R.; Moroder, L. Angew. Chem. Int. Ed. 2001, 40, 923.
51. Fisher, S.; Dunbrack, Jr. R. L.; Karplus, M. J. Am. Chem. Soc. 1994, 116, 11931.
52. Beausoleil, E.; Sharma, R.; Michnick, S. W.; Lubell, W. D. J. Org. Chem. 1998, 63, 6572.
53. Halab, L.; Lubell, W. D. J. Org. Chem. 1999, 64, 3312.
54. Albers, M. W.; Walsh, C. T.; Schreiber, S. L. J. Org. Chem. 1990, 55. 4984.

### Example 2

1. (a) Richardson, J. S. Adv. Protein Chem. 1981, 34, 167-339. (b) Wilmot, C. M.; Thornton, J. M. J. Mol. Biol. 1988, 203, 221-232. (c) Rose, G. D.; Gierasch, L. M.; Smith, J. A. Adv. Protein Chem. 1985, 37, 1-109.
2. (a) Creighton, T. E. Proteins: Structure and Molecular Properties. Freeman: New York, 1983. (b) Rizo, J.; Gierasch, L. Annu. Rev. Biochem. 1992, 61, 387-418.
3. Lewis, P. N.; Momany, F. A.; Scheraga, H. A. Proc. Nat. Acad. Sci., U.S.A. 1971, 68, 2293-2297.
4. (a) Branden, C.; Tooze, J. Introduction to Protein Structure; Garland Publishing: New York, 1991. (b) Stryer, L. Biochemistry, 4^{th} ed.; W.H. Freeman and Company: New York, 1999.
5. Reviewed in: (a) Fisher, G. Angew. Chem. Int. Ed. Engl. 1994, 33, 1415-1436. (b) Liu, J.; Chen. C.-M.; Walsh, C. T. Biochemistry 1991, 30, 2306-2310.
6. Johnson, M. E.; Lin, Z.; Padmanabhan, K.; Tulinsky, A.; Kahn, M. FEBS Lett. 1994, 337, 4-8.
7. Dugave, C.; Demange, L. Chem. Rev. 2003, 103, 2475-2532.
8. Halab, L.; Lubell, W. D. J. Am. Chem. Soc. 2002, 124, 2474-2484. And references therein.
9. An, S. S. A.; Lester C. C.; Peng, J.-L.; Li, Y.-J.; Rothwarf, D. M.; The inventorslker, E.; Thannhauser, T. W.; Zhang, L. S.; Tam, J. P.; Scheraga, H. A. J. Am. Chem. Soc. 1999, 121, 11558-11566.
10. (a) Didierjean, C.; Del Duca, V.; Benedetti, E.; Aubry, A.; Zouikri, M.; Marraud, M.; Boussard, G. J. Peptide Res. 1997, 50, 451-457. (b) Zouikri, M.; Vicherat, A.; Aubry, A.; Marraud, M.; Boussard, G. J. Peptide Res. 1998, 52, 19-26.
11. (a) Dumy, P.; Keller, M.; Ryan, D. E.; Rohwedder, B.; Wöhr, T.; Mutter, M. J. Am. Chem. Soc. 1997, 119, 918-925. (b) Kern, D.; Schutkowski, M.; Drakenberg, T. J. Am. Chem. Soc. 1997, 119, 8403-8408. (c) Keller, L.; Sager, C.; Dumy, P.; Schutkowski, M.; Fischer, G. S.; Mutter, M. J. Am. Chem. Soc. 1998, 120, 2714-2710.
12. Zhang, K.; Schweizer, F. Synlett 2005, 3111-3115.
13. Owens, N.; Braun, C.; Schweizer, F. J. Org. Chem., 2007, 72, 4635-4643.
14. This work will be published in another paper.
15. Jeannotte, G.; Lubell, W. D. J. Am. Chem. Soc. 2004, 126, 14334-14335.
16. (a) Walker, J. R.; Alshafie, G.; Nieves, N.; Ahrens, J.; Clagett-Dame, M.; Abou-Issa, H.; Curley, R. W. Bioorg. Med. Chem. 2006, 14, 3038-3048. (b) Sicheral, F.; Wittmann, V. Angew. Chem. Int. Ed. 2005, 44, 2096-2099.
17. The acid was treated with 2 equiv. iodomethane and 1.2 equiv. cesium carbonate in methanol and stirred for 1 hour to provide the corresponding methyl ester.
18. Frerot, E.; Coste, J.; Pantaloni, A.; Dufour, M. N.; Jouin, P. Tetrahedron, 1991, 47, 259-270.
19. Bürgi, H. B.; Dunitz, J. D.; Lehn, J. M.; Wipff, G. 1974, Tetrahedron 30, 1563-1572.
20. Runkel, L.; Neier, W.; Repinsky, R. B.; Karpusas, M.; Whitty, A.; Kimball, K.; Brickelmaier, M.; Muldowney, C.; Jones, W.; Goelz, S. E. Pharm. Res. 1998, 15, 641-649.
21. Kopple, K. D.; Ohnishi, M.; Go, A. J. Am. Chem. Soc. 1969, 91, 4264-4272.
22. Misicka, A.; Verheyden, P. M. F.; Van Binst, G. Lett. Pept. Sci. 1998, 5, 375-377.
23. Chakraborty, T. K.; Srinivasu, P.; Vengal Rao, R.; Kiran Kumar, S.; Kunwar, A. C. J. Org. Chem. 2004, 69, 7399-7402.
24. Leeflang, B. R.; Vliegenthart, J. F. G.; Kroon-Batenburg, L. M. J.; Eijck, B. P.; Kroon, J. Carbohydr. Res. 1992, 230, 41-61.

### Example 3

(1) Gellman, S. H. Acc. Chem. Res. 1998, 31, 173-180.
(2) Belec, L.; Slaminova, J.; Lubell, W. D. J. Med. Chem. 2000, 43, 1448-1455.
(3) An, S. S. A.; Lester, C. C.; Peng, J.; Li, Y.; Rothwarf, D. M.; Welker, E.; Thannhauser, T. W.; Zhang, L. S.; Tam, J. S.; Scheraga, H. A. J. Am. Chem. Soc. 1999, 121, 11558-11566.
(4) (a) Eberhardt, E. S.; Panasik, Jr. N.; Raines, R. T. J. Am. Chem. Soc. 1996, 118, 12261-12266. (b) Improta, R.; Benzi C.; Barone, V. J. Am. Chem. Soc. 2001, 123, 12568-12577. (c) Song, Il K.; Kang, Y. K. J. Phys. Chem. B 2005, 109, 16982-16987
(5) Hinderaker, M. P.; Raines, R. T. Protein Sci. 2003, 12, 1188-1194.
(6) (a) Wu, W. J.; Raleigh, D. P. Biopolymers, 1998, 45, 381-394. (b) Thomas, K. M.; Naduthambi, D.; Zondlo, N. J. J. Am. Chem. Soc. 2006, 128, 2216-2217. (c) Halab, L.; Lubell, W. D. J. Am. Chem. Soc. 2002, 124, 2474-2484.
(7) Beausoleil, E.; Lubell, W. D. J. Am. Chem. Soc. 1996, 118, 12902-12908. And references therein.
(8) Kakinoki, S.; Hirano, Y.; Oka, M. Polym. Bull. (Berlin) 2005, 53, 109-115.
(9) Reddy, K. V. R.; Yedery, R. D.; Aranha, C. Int. J. Antimicrobial Agents 2004, 24, 536-547.
(10) Cooper, J. B.; Chen, J. A.; Van Holst, G. J.; Varner, J. E. TIBS, 1987, 12, 24-27. And references therein.
(11) (a) Baldwin, J. E.; Adlington, R. M.; Gollins, D. W.; Godfrey, C. R. A. Tetrahedron 1995, 51, 5169-5180; (b) Hashimoto, K.; Yamamoto, O.; Horikawa, M.; Ohfune, Y.; Shirahama, H. Bioorg. Med. Chem. Lett. 1994, 4, 1851-1854.
(12) (a) Bridges, R. J.; Stanley, M. S.; Anderson, M. W.; Cotman, C. W.; Chamberlin, A. R. J. Med. Chem. 1991, 34, 717-725. (b) Tsai, C.; Schneider, J. A.; Lehmann, J. Neurosci. Lett. 1988, 92, 298-302.
(13) (a) Kolodziej, S. A.; Nikiforovich, G. V.; Skeean, R.; Lignon, M.-F.; Martinez, J.; Marshall, G. R. J. Med. Chem. 1995, 38, 137-149. (b) Holladay, M. W.; Lin, C. W.; May, C. S.; Garvey, D. S.; Witte, D. G.; Miller, T. R.; Wolfram, C. A. W.; Nadzan, A. M. J. Med. Chem. 1991, 34, 455-457. (c) Plucinska, K.; Kataoka, T.; Yodo, M.; Cody, W. L.; He, J. X.; Humblet, C.; Lu, G. H.; Lunney, E.; Major, T. C.; Panek, R. L.; Schelkun, P.; Skeean, R.; Marshall, G. R. J. Med. Chem. 1993, 36, 1902-1913.
(14) Ghose, A. K.; Logan, M. E.; Treasurywala, A. M.; Wang, H.; Wahl, R. C; Tomczuk, B. E.; Gowravaram, M. R.; Jaeger, E. P.; Wendoloski, J. J. J. Am. Chem. Soc. 1995, 117, 4671-4682.
(15) (a) Mosberg, H. I.; Kroona, H. B. J. Med. Chem. 1992, 35, 4498-4500. (b) Mosberg, H. I.; Lomize, A. L.; Wang, C.; Kroona, H.; Heyl, D. L.; Sobczyk-Kojiro, K.; Ma, W.; Mousigian, C.; Porreca, F. J. Med. Chem. 1994, 37, 4371-4383.
(16) Chung, J. Y. L.; Wasicak, J. T.; Arnold, W. A.; May, C. S.; Nadzan, A. M.; Holladay, M. W. J. Org. Chem. 1990, 55, 270-275.
(17) Sharma, R.; Lubell, W. D. J. Org. Chem. 1996, 61, 202-209.
(18) Reviews: (a) Dondoni A.; Marra, A. Chem. Rev. 2000, 100, 4395-4421. (b) Schweizer, F. Angew. Chem. Int. Ed. 2002. 41, 230-253. (c) Gruner, S. W.; Locardi, E.; Lohof, E.; Kessler, H. Chem. Rev. 2002, 102, 491-514.
(19) (a) Owens, N.; Braun, C.; Schweizer, F. J. Org. Chem., 2007, 72, 4635. (b) Zhang, K.; Schweizer, F. Synlett. 2005, 3111-3115. (c) Cipolla, L.; Redaelli, C.; Nicotra, F. Lett. Drug Des. Discov. 2005, 2, 291-293.
(20) Zhang, K., Mondal, D., Zhanel, G. G. and Schweizer, F. Carbohydr. Res. 2008, 343, 1644-1652
(21) Haubner, R.; Wester, H-J.; Burkhart, F.; Senekowitsch-Schmidtke, R.; Weber, W.; Goodman, S. L.; Kessler, H.; Schwaiger, M. J. Nuclear. Med. 2001, 42, 326-336.
(22) (a) Kuemin, M.; Sonntag, L.-S.; Wennemers, H. J. Am. Chem. Soc. 2007, 129, 466-467. (b) Shi, Z.; Chen, K.; Liu, Z.; Kallenbach, N. R. Chem. Rev. 2006, 106, 1877-1897.
(23) Grotenbreg, G. M.; Timmer, M. S. M.; Llamas-Saiz, A. L.; Verdoes, M.; Van der Marel, G. A.; Van Raaij, M. J.; Overkleeft, H. S.; Overhand, M. J. Am. Chem. Soc. 2004, 126, 3444-3446.
(24) Chakraborty, T. K.; Jayaprakash, S.; Diwan, P. V.; Nagaraj, R.; Jampani, S. R. B.; Kunwar, A. C. J. Am. Chem. Soc. 1998, 120, 12962-12963.
(25) It has been reported that substitution of D-proline by cis-3-hydroxy-D-proline (HypC3-OH) in the sequence Boc-Leu-Pro-Gly-Leu-NHMe resulted in novel pseudo β-turn-like nine-membered ring structure involving an intramolecular LeuNH→HypC3-OH hydrogen bond. Chakraborty, T. K.; Srinivasu, P.; Vengal Rao, R.; Kiran Kumar, S.; Kunwar, A. C. J. Org. Chem. 2004, 69, 7399-7402.
(26) Gueyrard, D.; Haddoub, R.; Salem, A.; Bacar, N. S.; Goekjian, P. G. Synlett 2005, 520-522.
(27) (a) Schweizer, F.; Inazu, T. Org. Lett. 2001, 3, 4115-4118. (b) Zhang, K.; Wang, J.; Sun, Z.; Huang, H-D; Schweizer, F. Synlett 2007, 239-242.
(28) Dillon, M. P.; Maag, H.; Muszyski, D. M. Tetrahedron Lett. 1995, 36, 5469-5470.
(29) A 40 ms Gaussian pulse with a 560 ms mixing time was used.
(30) Huang, S. K.; Omura, K.; Swern, D. J. Org. Chem. 1976, 41, 3329-3331.
(31) Boeykens, M.; Kimpe, N. D.; Tehrani, K. A. J. Org. Chem. 1994, 59, 6973-6985.
(32) Chérest, M.; Felkin, H.; Prudent, N. Tetrahedron Lett. 1968, 9, 2199-2204.
(33) Davies, S. G.; Nicholson, R. L.; Price, P. D.; Roberts, P. M.; Smith, A. D. Synlett 2004, 5, 901-903.
(34) GOESY is a selective 1D NOESY experiment, see: Stonehouse, J., Adell, P., Keeler, J., Shaka, A. J. J. Am. Chem. Soc. 1994, 116, 6037-6038.
(35) Taylor, C. M.; Hardré, R.; Edwards, P. J. B.; Park, J. H. Org. Lett. 2003, 5, 4413-4416. And references therein.
(36) Dugave, C.; Demange, L. Chem. Rev. 2003, 103, 2475-2532.
(37) (a) Jenkins, C. L.; Bretscher, L. E.; Guzei, I. A.; Raines, R. T. J. Am. Chem. Soc. 2003, 125, 6422-6427. (b) Taylor, C. M.; Hardré, R.; Edwards, P. J. B. J. Org. Chem. 2005, 70, 1306-1315.
(38) Leeflang, B. R.; Vliegenthart, J. F. G.; Kroon-Batenburg, L. M. J.; Eijck, B. P.; Kroon, J. Carbohydr. Res. 1992, 230, 41-61.
(39) Wilmot, C. M.; Thornton, J. M. J. Mol. Biol. 1988, 203, 221-232.
(40) Marraud, M.; Aubry, A. Int. J. Peptide Protein Res. 1984, 23, 123-133.

1. A compound of formula (I) 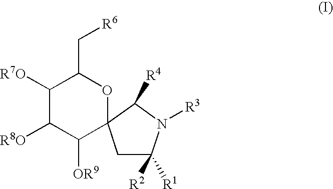
wherein:
one of R¹ and R² is —CH₂OH or CH₂OR⁵, wherein R⁵ is a hydroxy protecting group, and the other of R¹ and R² is —H;
R³ and R⁴ are, independently, —H, —COOMe, —CONHMe, an amine protecting group, an amino acid, a protected amino acid, or a peptide; and
R⁶ is —H, —OH, —NH₂, —N₃, a protected hydroxyl group, or a protected amine group;
R⁷ through R⁹ are independently —H or R⁵, wherein R⁵ is a hydroxy protecting group.

2. The compound of claim 1, wherein R⁵ is a hydroxy protecting group selected from an alkyl group, an alkenyl group, an alkanoyl group; an alkoxycarbonyl group; an alkenyloxycarbonyl group, an aryl-alkoxycarbonyl group, a nitrobenzyloxycarbonyl group, a trialkylsilyl group, or an aryl-alkyl group.

3. The compound of claim 1, wherein R⁵ is a methoxymethyl group.

4. The beta-turn mimetic compound of claim 1, wherein the peptide at either R³ and R⁴ comprises 10 or less amino acids.

5. The beta-turn mimetic compound of claim 4, wherein the peptide at either R³ and R⁴ comprises 6 or less amino acids.

6. The beta-turn mimetic compound of claim 5, wherein the peptide at either R³ and R⁴ is a dipeptide.

7. The compound of claim 1, wherein the compound is further defined as an analog of a bioactive peptide.

8. The compound of claim 7, wherein the bioactive peptide is an antimicrobial peptide.

9. The compound of claim 8, wherein the antimicrobial peptide comprises a D- or L-proline unit.

10. The compound of claim 9, wherein the antimicrobial peptide is a gramicidin, a tachyplesin, an indolicidin, an arenicin, a tritrpticin, or a tigerinin.

11. The compound of claim 1, further defined as a beta-turn mimetic.

12. The beta-turn mimetic compound of claim 11, wherein R¹⁵ is an amino acid and R¹⁷ is a dipeptide.

13. The beta-turn mimetic compound of claim 11, wherein R¹⁵ is a protected amino acid and R¹⁷ is a dipeptide.

14. The beta-turn mimetic compound of claim 11, wherein the beta-turn mimetic is further defined as having one of the following structures:

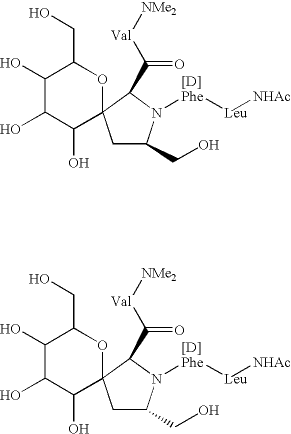

15. A method of mimicking a beta-turn in a peptide comprising:
replacing an amino acid within a native beta-turn structure of the peptide with a spirocyclic proline hybrid formula (II): 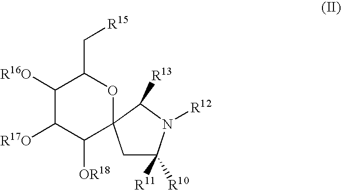
wherein:
one of R¹⁰ and R¹¹ is —CH₂OH or CH₂OR¹⁴, where R¹⁴ is a hydroxy protecting group, and the other of R¹⁰ and R¹¹ is —H;
R¹² and R¹³ are, independently, —H, —COOH, —COOMe, —CONHMe, an amine protecting group, or a carboxy protecting group; and
R¹⁵ is —H, —OH, —NH₂, —N₃, a methoxymethyl ether, a protected hydroxyl group, or a protected amine group;
R¹⁶ through R¹⁸ are independently —H or R¹⁴ where R¹⁴ is a hydroxy protecting group.

16. The method of claim 15, wherein R¹⁴ is a hydroxy protecting group selected from an alkyl group, an alkenyl group, an alkanoyl group; an alkoxycarbonyl group; an alkenyloxycarbonyl group, an aryl-alkoxycarbonyl group, a nitrobenzyloxycarbonyl group, a trialkylsilyl group, and aryl-alkyl group.

17. The method of claim 15, wherein R¹⁴ is a methoxymethyl group.

18. The method of claim 15, wherein the peptide is a bioactive peptide.

19. The method of claim 18, wherein the peptide is an antimicrobial peptide.

20. The method of claim 19, wherein the antimicrobial peptide comprises a D- or L-proline unit.

21. The method of claim 20, wherein the antimicrobial peptide is a gramicidin, a tachyplesin, an indolicidin, an arenicin, a tritrpticin, or a tigerinin.

22. A method of synthesizing a beta-turn mimetic of claim 1, comprising:
blocking the hydroxy and amine groups of a spirocyclic proline hybrid of formula (II): 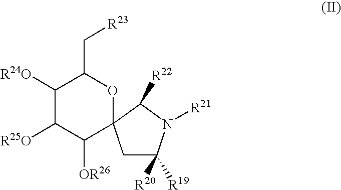
wherein:
one of R¹⁹ and R²⁰ is —CH₂OH and the other of R¹⁹ and R²⁰ is —H;
R²¹ and R²² are, independently, —H, —COOH, —COOMe, —CONHMe, an amine protecting group, or a carboxy protecting group; and
R²³ is —H, —OH, —NH₂, —N₃, a methoxymethyl ether, a protected hydroxyl group, or a protected amine group;
R²⁴ through R²⁶ are independently —H or a hydroxy protecting group.
hydrolyzing or displacing the carboxy terminal ester of hydroxyproline;
optionally reacting the hydrolyzed carboxy terminal group with a protected amino acid or a peptide;
deblocking the nitrogen of the hydroxyproline ring;
optionally acetylating the nitrogen of the hydroxyproline ring, or coupling the nitrogen of the hydroxyproline ring to a protected amino acid or a peptide; and
optionally deblocking one or more protected amino acids or hydroxyl groups.
